SEQUENCE LISTING (58 sequences)

SEQ ID NO: 1 - protein, 7 aa, synthetic construct
SNSYYWG

SEQ ID NO: 2 - protein, 16 aa, synthetic construct
SIYYSGSTYYNPSLKS

SEQ ID NO: 3 - protein, 19 aa, synthetic construct
VRRVAGRGDSYYYYYGMDV

SEQ ID NO: 4 - protein, 11 aa, synthetic construct
RASQGISRWLA

SEQ ID NO: 5 - protein, 7 aa, synthetic construct
AASSLQS

SEQ ID NO: 6 - protein, 9 aa, synthetic construct
QQAISHPWT

SEQ ID NO: 7 - protein, 6 aa, synthetic construct
SGYYWG

SEQ ID NO: 8 - protein, 16 aa, synthetic construct
SIYHSGNTYYNPSLKS

SEQ ID NO: 9 - protein, 17 aa, synthetic construct
VPRYSRGDAYYYYGMDV

SEQ ID NO: 10 - protein, 11 aa, synthetic construct
RASQSISSWLA

SEQ ID NO: 11 - protein, 7 aa, synthetic construct
KASSLES

SEQ ID NO: 12 - protein, 10 aa, synthetic construct
QQVKIYPPYT

SEQ ID NO: 13 - protein, 6 aa, synthetic construct
SGYYWG

SEQ ID NO: 14 - protein, 16 aa, synthetic construct
SIYHSGNTYYNPSLKS

SEQ ID NO: 15 - protein, 17 aa, synthetic construct
VPRYSRGDAYYYYGMDV

SEQ ID NO: 16 - protein, 11 aa, synthetic construct
RASQSIGSWLA

SEQ ID NO: 17 - protein, 7 aa, synthetic construct
KASSLES

SEQ ID NO: 18 - protein, 9 aa, synthetic construct
QQSGPYPWT

SEQ ID NO: 19 - protein, 6 aa, synthetic construct
SGYYWG

SEQ ID NO: 20 - protein, 16 aa, synthetic construct
SIYHSGNTYYNPSLKS

SEQ ID NO: 21 - protein, 17 aa, synthetic construct
VPRYSRGDAYYYYGMDV

SEQ ID NO: 22 - protein, 12 aa, synthetic construct
RASQSVRSSYLA

SEQ ID NO: 23 - protein, 7 aa, synthetic construct
GASSRAT

SEQ ID NO: 24 - protein, 8 aa, synthetic construct
QQAGLVFT

SEQ ID NO: 25 - protein, 5 aa, synthetic construct
NYAIS

SEQ ID NO: 26 - protein, 17 aa, synthetic construct
GIIPIFGTANYAQKFQG

SEQ ID NO: 27 - protein, 18 aa, synthetic construct
APRSARGDMYYYYYGMDV

SEQ ID NO: 28 - protein, 11 aa, synthetic construct
RASQSIGSWLA

SEQ ID NO: 29 - protein, 7 aa, synthetic construct
KASSLES

SEQ ID NO: 30 - protein, 9 aa, synthetic construct
QQYGRYPWT

SEQ ID NO: 31 - protein, 129 aa, synthetic construct
ELQLQESGPGLVKPSETLSLTCTVSGGSISSNSYYWGWIRQPPGKGLEWIGSIYYSGSTYYNPSLKSRVTISVDTSKNQFSLKLSSVTAADTAVYYCARVRRVAGRGDSYYYYYGMDVWGQGTTVTVSS

SEQ ID NO: 32 - protein, 107 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQAISHPWTFGGGTKVEIK

SEQ ID NO: 33 - protein, 126 aa, synthetic construct
EVQLQESGPGLVKPSETLSLTCAVSGYSISSGYYWGWIRQPPGKGLEWIGSIYHSGNTYYNPSLKSRVTISVDTSKNQFSLKLSSVTAADTAVYYCARVPRYSRGDAYYYYGMDVWGQGTTVTVSS

SEQ ID NO: 34 - protein, 108 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQVKIYPPYTFGGGTKVEIK

SEQ ID NO: 35 - protein, 126 aa, synthetic construct; identical to SEQ ID NO: 33

SEQ ID NO: 36 - protein, 107 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQSGPYPWTFGGGTKVEIK

SEQ ID NO: 37 - protein, 126 aa, synthetic construct; identical to SEQ ID NO: 33

SEQ ID NO: 38 - protein, 107 aa, synthetic construct
EIVLTQSPGTLSLSPGERATLSCRASQSVRSSYLAWYQQKPGQAPRLLIYGASSRATGIPDRFSGSGSGTDFTLTISRLEPEDFAVYYCQQAGLVFTFGGGTKVEIK

SEQ ID NO: 39 - protein, 127 aa, synthetic construct
EVQLVQSGAEVKKPGSSVKVSCKASGGTFSNYAISWVRQAPGQGLEWMGGIIPIFGTANYAQKFQGRVTITADESTSTAYMELSSLRSEDTAVYYCARAPRSARGDMYYYYYGMDVWGQGTTVTVSS

SEQ ID NO: 40 - protein, 107 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYGRYPWTFGGGTKVEIK

SEQ ID NO: 41 - protein, 330 aa, synthetic construct [sequence omitted]

SEQ ID NO: 42 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 330 aa, synthetic construct
  VARIANT 180: note = Any amino acid other than N
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 107 aa, synthetic construct [sequence omitted]

SEQ ID NO: 51 - protein, 20 aa, synthetic construct
MSVPTQVLGLLLLWLTDARC

SEQ ID NO: 52 - protein, 19 aa, synthetic construct
MEWSWVFLFFLSVTTGVHS

SEQ ID NO: 53 - protein, 5 aa, synthetic construct
GGGGS

SEQ ID NO: 54 - protein, 25 aa, synthetic construct
  VARIANT 1..25: note = This sequence may encompass 1-5 GGGGS repeating units
GGGGSGGGGSGGGGSGGGGSGGGGS

SEQ ID NO: 55 - protein, 15 aa, synthetic construct
GGGGSGGGGSGGGGS

SEQ ID NO: 56 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 57 - protein, 12 aa, synthetic construct
GGGSGGGSGGGS

SEQ ID NO: 58 - protein, 6 aa, synthetic construct
HHHHHH

(19) United States
(12) Patent Application Publication
(10) Pub. No.: US 2025/0313635 A1
(43) Pub. Date: Oct. 9, 2025

(54) ANTI-INTEGRIN ANTIBODIES AND USES THEREOF

(71) Applicant: Merck Sharp & Dohme LLC, Rahway, NJ (US)

(72) Inventors: Masahisa Handa, Berkeley, CA (US); Josephine Lau Johnson, San Leandro, CA (US); Ashmita Saigal, Foster City, CA (US); Tao Wang, North Reading, MA (US); Ji Zhang, San Jose, CA (US)

(73) Assignee: Merck Sharp & Dohme LLC, Rahway, NJ (US)

(21) Appl. No.: 18/864,678

(22) PCT Filed: May 8, 2023
(86) PCT No.: PCT/US2023/021298

§ 371 Date: Nov. 11, 2024

Related U.S. Application Data
(60) Provisional application No. 63/341,056, filed on May 12, 2022.

(51) Int. Cl. C07K 16/28

(52) U.S. Cl. CPC C07K 16/2839; C07K 2317/33; C07K 2317/76; C07K 2317/92

(57) ABSTRACT

The integrin family of cell adhesion molecules has emerged as key mediators of tissue fibrosis. A pharmacological inhibitor of multiple integrin subtypes is required to produce meaningful effects on delaying or inhibiting the progression of fibrosis. Monoclonal antibodies recognizing multiple integrins with potent neutralizing activity and having human and mouse cross-reactivity are described. In particular, monoclonal antibodies that bind human αvβ1, αvβ3, αvβ5, αvβ36, αvβ38, and α5β1 integrins and mouse αvβ1, αvβ3, αvβ5, αvβ6, and αvβ8 integrins are described.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Oct. 28, 2022, is named 25393-WO-PCT_SL.XML and is 55.1 bytes in size.

### BACKGROUND OF THE INVENTION

### Field of the Invention

The present invention relates to monoclonal antibodies recognizing multiple integrins with potent neutralizing activity and having human and mouse cross-reactivity. In particular, the present inventions provide monoclonal antibodies that bind human αvβ1, αvβ3, αvβ5, αvβ6, αvβ8, and α5β1 integrins and mouse αvβ1, αvβ3, αvβ5, αvβ6, and αvβ8 integrins.

### Description of Related Art

Idiopathic pulmonary fibrosis (IPF) is a chronic, fibrosing interstitial lung disease with unknown etiology. Patients suffer from chronic coughs and deteriorating breathing difficulties. The median survival is 2.5-3.5 years from diagnosis. Despite the severe clinical impact, there are limited treatment options for lung fibrosis. In 2014, the FDA approved the use of Pirfenidone (ESBRIET, Genentech) and Nintedanib (OFEV, Boehringer Ingelheim) in IPF patients. Both drugs slow the decline of lung function as measured by the decrease of FVC (forced vital capacity), a surrogate endpoint measurement (Wilson & Raghu, Eur. Respir. J. 46, 883-886 (2015)). However, neither drug appears to stop disease progression, relieve breathing difficulty, or substantially improve patient survival. There is an unmet medical need to develop new IPF therapies that bring clinically meaningful efficacy to patients.

In recent years, the integrin family of cell adhesion molecules has emerged as key mediators of tissue fibrosis. Among the 24 known integrin heterodimers, five αv integrins (αvβ1, αvβ3, αvβ5, αvβ6, and αvβ8) transduce mechanical and biochemical signals from fibrotic extracellular matrix into the cell, activate latent TGFβ, and subsequently modulate fibroblast adhesion, migration, and growth (Hynes, Cell 110, 673-687 (2002)). The αv integrins primarily interact with the RGD (Arginine-Glycine-Aspartic acid) peptide present in fibronectin and vitronectin (αvβ1, αvβ3, and αvβ5), or with the RGD motif of the TGFβ latency—associated peptide (LAP) (αvβ1, αvβ6, and αvβ8) (Hynes, Cell 110, 673-687 (2002); Munger et al., Cell 96, 319-328 (1999); Kitamura et al., J. Clin. Invest. 121, 2863-2875 (2011); Reed, N. I. et al., Sci. Transl. Med. 7, 288 (2015)). As a result, αv integrins play a key role in the regulation of TGFβ signaling (Henderson, et al., Nat. Med. 19, 1617-1624 (2013). Dysregulated expression and response to TGFβhas been implicated in a wide variety of disease processes including fibrotic disease and chronic inflammation (Akhurst & Hata, Nat. Rev. Drug. Discov. 11, 790-811 (2012)). The epithelium-specific αvβ6 integrin binds to latent TGFβ and facilitates release of the mature cytokine, a process called TGFβ activation (Munger et al., Cell 96, 319-328 (1999); Dong et al., Nat. Struct. Mol. Biol. 21, 1091-1096 (2014). Deletion of 36 integrin in mice is protective against bleomycin-induced lung fibrosis (Munger et al., Cell 96, 319-328 (1999)), and an anti-mouse αvβ6 antibody has shown similar beneficial effects in preclinical animal studies (Horan et al., Am. J. Respir. Crit. Care. Med. 177, 56-65 (2008)). An αvβ6 antibody (BG00011/Biogen) and a small molecule inhibitor GSK3008348 were used in clinical trials of IPF patients (clinicaltrials.gov identifier NCT03573505, NCT03069989) (Maden et al., Eur. J. Clin. Pharmacol. 74, 701-709 (2018)). αvβ1, the less-known member of the integrin family, was recently shown to be highly expressed in activated fibroblasts and modulate lung and liver fibrosis in mice (Reed, N. I. et al., Sci. Transl. Med. 7, 288 (2015)). Additionally, αvβ8 integrin, another regulator of latent TGFβ activation, modulates chemokine secretion and dendritic cell trafficking (Kitamura et al., J. Clin. Invest. 121, 2863-2875 (2011); Mu et al., J. Cell. Biol. 157, 493-507 (2002)). β8 knockout mice and mice treated with a blocking D8 antibody are protected against airway inflammation and fibrosis (Kitamura et al., J. Clin. Invest. 121, 2863-2875 (2011); Minagawa et al., Sci. Transl. Med. 6, 241-279 (2014)). Although the role of a pan-av inhibitor has not been extensively tested in the clinic for lung indications, evidence from multiple lines of work suggest that modulating αv integrin activity will lead to anti-fibrotic effects in various tissues. A report by Henderson et al. demonstrated that depletion of αv integrin in myofibroblasts lead to protection against hepatic fibrosis induced by carbon tetrachloride, renal fibrosis induced by unilateral ureter obstruction, and lung fibrosis induced by bleomycin (Henderson, et al., Nat. Med. 19, 1617-1624 (2013)). Furthermore, a small molecule RGD mimetic CWHM12 similarly attenuates liver and lung fibrosis (Henderson, et al., Nat. Med. 19, 1617-1624 (2013)). The complexity of the integrins and their role in the progression of the disease suggest that a pharmacological inhibitor of multiple integrin subtypes would be required to produce meaningful effects on delaying or inhibiting the progression of fibrosis. Interestingly, recent genome-wide association analysis of 400,102 individuals identifies an association of reduced αv gene expression with increased lung function (Shrine et al., Nat. Genet. 51, 481-493 (2019)).

Lung fibrosis, a devastating disease with limited treatment options and a prognosis that is worse than most types of cancer, currently presents a huge unmet medical need.

### BRIEF SUMMARY OF THE INVENTION

The present invention provides several potent integrin binders with unique human and mouse cross-species affinity. The integrin binders of the present invention are pan-av integrin binders comprising chimeric or fully human antibodies or antigen binding fragments thereof that specifically bind human integrins αvβ1, αvβ3, αvβ5, αvβ6, and αvβ8 integrins and mouse integrins αvβ1, αvβ3, αvβ5, αvβ6, and αvβ8 integrins as determined in the cell-based binding assay (CELISA) as disclosed in the General Methods herein. Exemplary integrin binders include antibodies Ab-29, Ab-30, Ab-31, Ab-32, and Ab-33 disclosed herein. Antibodies Ab-29, Ab-30, Ab-31, and Ab-32 are further capable of binding bind human integrin αvβ1 as determined in the cell-based binding assay (CELISA) as disclosed in the General Methods herein. As disclosed herein, the integrin binders of the present invention may be useful for treatment of cancers and/or fibrosis. In a particular embodiment, the integrin binders may be used in treatment for idiopathic pulmonary fibrosis.

The present invention provides an integrin binder comprising the six complementarity determining regions (CDRs) of an antibody having a heavy chain variable domain (V_{H}) comprising the amino acid sequence set forth in SEQ ID NO: 31 and a light chain 25 variable domain (V_{L}) comprising the amino acid sequence set forth in SEQ ID NO: 32, wherein the CDRs are defined using the Kabat, Chothia, AbM, ImMunoGeneTics (IMGT), or Contact numbering scheme.

The present invention provides an integrin binder comprising the six CDRs of an antibody having a V_{H} comprising the amino acid sequence set forth in SEQ ID NO: 33 and a V_{L} comprising the amino acid sequence set forth in SEQ ID NO: 34, wherein the CDRs are defined using the Kabat, Chothia, AbM, ImMunoGeneTics (IMGT), or Contact numbering scheme.

The present invention provides an integrin binder comprising the six CDRs of an antibody having a V_{H} comprising the amino acid sequence set forth in SEQ ID NO: 35 and a V_{L} comprising the amino acid sequence set forth in SEQ ID NO: 36, wherein the CDRs are defined using the Kabat, Chothia, AbM, ImMunoGeneTics (IMGT), or Contact numbering scheme.

The present invention provides an integrin binder comprising the six CDRs of an antibody having a V_{H} comprising the amino acid sequence set forth in SEQ ID NO: 37 and a V_{L} comprising the amino acid sequence set forth in SEQ ID NO: 38, wherein the CDRs are defined using the Kabat, Chothia, AbM, ImMunoGeneTics (IMGT), or Contact numbering scheme.

The present invention provides an integrin binder comprising the six CDRs of an antibody having a V_{H} comprising the amino acid sequence set forth in SEQ ID NO: 39 and a V_{L} comprising the amino acid sequence set forth in SEQ ID NO: 40, wherein the CDRs are defined using the Kabat, Chothia, AbM, ImMunoGeneTics (IMGT), or Contact numbering scheme.

The present invention further provides an integrin binder comprising:
(a) a V_{H} comprising a CDR 1 comprising the amino acid sequence set forth in SEQ ID NO: 1, a CDR-2 comprising the amino acid sequence set forth in SEQ ID NO: 2, and a CDR 3 comprising the amino acid sequence set forth in SEQ ID NO: 3; and
(b) a V_{L} comprising a CDR 1 comprising the amino acid sequence set forth in SEQ ID NO: 4, a CDR 2 comprising the amino acid sequence set forth in SEQ ID NO: 5, and a CDR 3 comprising the amino acid sequence set forth in SEQ ID NO: 6.

The present invention further provides an integrin binder comprising:
(a) a V_{H} comprising a CDR 1 comprising the amino acid sequence set forth in SEQ ID NO: 7, a CDR-2 comprising the amino acid sequence set forth in SEQ ID NO: 8, and a CDR 3 comprising the amino acid sequence set forth in SEQ ID NO: 9; and
(b) a V_{L} comprising a CDR 1 comprising the amino acid sequence set forth in SEQ ID NO: 10, a CDR 2 comprising the amino acid sequence set forth in SEQ ID NO: 11, and a CDR 3 comprising the amino acid sequence set forth in SEQ ID NO: 12.

The present invention further provides an integrin binder comprising:
(a) a V_{H} comprising a CDR 1 comprising the amino acid sequence set forth in SEQ ID NO: 13, a CDR-2 comprising the amino acid sequence set forth in SEQ ID NO: 14, and a CDR 3 comprising the amino acid sequence set forth in SEQ ID NO: 15; and
(b) a V_{L} comprises a CDR 1 comprising the amino acid sequence set forth in SEQ ID NO: 16, a CDR 2 comprising the amino acid sequence set forth in SEQ ID NO: 17, and a CDR 3 comprising the amino acid sequence set forth in SEQ ID NO: 18.

The present invention further provides an integrin binder comprising:
(a) a V_{H} comprising a CDR 1 comprising the amino acid sequence set forth in SEQ ID NO: 19, a CDR-2 comprising the amino acid sequence set forth in SEQ ID NO: 20, and a CDR 3 comprising the amino acid sequence set forth in SEQ ID NO: 21; and
(b) a V_{L} comprises a CDR 1 comprising the amino acid sequence set forth in SEQ ID NO: 22, a CDR 2 comprising the amino acid sequence set forth in SEQ ID NO: 23, and a CDR 3 comprising the amino acid sequence set forth in SEQ ID NO: 24.

The present invention further provides an integrin binder comprising:
(a) a V_{H} comprising a CDR 1 comprising the amino acid sequence set forth in SEQ ID NO: 25, a CDR-2 comprising the amino acid sequence set forth in SEQ ID NO: 26, and a CDR 3 comprising the amino acid sequence set forth in SEQ ID NO: 27; and
(b) a V_{L} comprises a CDR 1 comprising the amino acid sequence set forth in SEQ ID NO: 28, a CDR 2 comprising the amino acid sequence set forth in SEQ ID NO: 29, and a CDR 3 comprising the amino acid sequence set forth in SEQ ID NO: 30.

The present invention further provides an integrin binder comprising:
(a) a heavy chain variable domain (V_{H}) comprising an amino acid sequence with at least 90% identity to the amino acid sequence set forth in SEQ ID NO: 31 and a light chain variable domain (V_{L}) comprising an amino acid sequence with at least 90% identity to the amino acid sequence set forth in SEQ ID NO: 32, wherein the V_{H} comprises a CDR 1 comprising the amino acid sequence set forth in SEQ ID NO: 1, a CDR-2 comprising the amino acid sequence set forth in SEQ ID NO: 2, and a CDR 3 comprising the amino acid sequence set forth in SEQ ID NO: 3, and the V_{L} comprises a CDR 1 comprising the amino acid sequence set forth in SEQ ID NO: 4, a CDR 2 comprising the amino acid sequence set forth in SEQ ID NO: 5, and a CDR 3 comprising the amino acid sequence set forth in SEQ ID NO: 6;
(b) a V_{H} comprising an amino acid sequence with at least 90% identity to the amino acid sequence set forth in SEQ ID NO: 33 and a V_{L} comprising an amino acid sequence with at least 90% identity to the amino acid sequence set forth in SEQ ID NO: 34, wherein the V_{H} comprises a CDR 1 comprising the amino acid sequence set forth in SEQ ID NO: 7, a CDR-2 comprising the amino acid sequence set forth in SEQ ID NO: 8, and a CDR 3 comprising the amino acid sequence set forth in SEQ ID NO: 9, and the V_{L} comprises a CDR 1 comprising the amino acid sequence set forth in SEQ ID NO: 10, a CDR 2 comprising the amino acid sequence set forth in SEQ ID NO: 11, and a CDR 3 comprising the amino acid sequence set forth in SEQ ID NO: 12;
(c) a V_{H} comprising an amino acid sequence with at least 90% identity to the amino acid sequence set forth in SEQ ID NO: 35 and a V_{L} comprising an amino acid sequence with at least 90% identity to the amino acid sequence set forth in SEQ ID NO: 36, wherein the V_{H} comprises a CDR 1 comprising the amino acid sequence set forth in SEQ ID NO: 13, a CDR-2 comprising the amino acid sequence set forth in SEQ ID NO: 14, and a CDR 3 comprising the amino acid sequence set forth in SEQ ID NO: 15, and the V_{L} comprises a CDR 1 comprising the amino acid sequence set forth in SEQ ID NO: 16, a CDR 2 comprising the amino acid sequence set forth in SEQ ID NO: 17, and a CDR 3 comprising the amino acid sequence set forth in SEQ ID NO: 18;
(d) a V_{H} comprising an amino acid sequence with at least 90% identity to the amino acid sequence set forth in SEQ ID NO: 37 and a V_{L} comprising an amino acid sequence with at least 90% identity to the amino acid sequence set forth in SEQ ID NO: 38, wherein the V_{H} comprises a CDR 1 comprising the amino acid sequence set forth in SEQ ID NO: 19, a CDR-2 comprising the amino acid sequence set forth in SEQ ID NO: 20, and a CDR 3 comprising the amino acid sequence set forth in SEQ ID NO: 21, and the V_{L} comprises a CDR 1 comprising the amino acid sequence set forth in SEQ ID NO: 22, a CDR 2 comprising the amino acid sequence set forth in SEQ ID NO: 23, and a CDR 3 comprising the amino acid sequence set forth in SEQ ID NO: 24; or
(e) a V_{H} comprising an amino acid sequence with at least 90% identity to the amino acid sequence set forth in SEQ ID NO: 39 and a V_{L} comprising an amino acid sequence with at least 90% identity to the amino acid sequence set forth in SEQ ID NO: 40, wherein the V_{H} comprises a CDR 1 comprising the amino acid sequence set forth in SEQ ID NO: 25, a CDR-2 comprising the amino acid sequence set forth in SEQ ID NO: 26, and a CDR 3 comprising the amino acid sequence set forth in SEQ ID NO: 27, and the V_{L} comprises a CDR 1 comprising the amino acid sequence set forth in SEQ ID NO: 28, a CDR 2 comprising the amino acid sequence set forth in SEQ ID NO: 29, and a CDR 3 comprising the amino acid sequence set forth in SEQ ID NO: 30.

In a further embodiment, the present invention provides an integrin binder comprising: (a) a heavy chain variable domain (V_{H}) comprising the amino acid sequence set forth in SEQ ID NO: 31 and a light chain variable domain (V_{L}) comprising the amino acid sequence set forth in SEQ ID NO: 32; (b) a V_{H} comprising the amino acid sequence set forth in SEQ ID NO: 33 and a V_{L} comprising the amino acid sequence set forth in SEQ ID NO: 34; (c) a V_{H} comprising the amino acid sequence set forth in SEQ ID NO: 35 and a V_{L} comprising the amino acid sequence set forth in SEQ ID NO: 36; (d) a V_{H} comprising the amino acid sequence set forth in SEQ ID NO: 37 and a V_{L} comprising the amino acid sequence set forth in SEQ ID NO: 38; or (e) a V_{H} comprising the amino acid sequence set forth in SEQ ID NO: 39 and a V_{L} comprising the amino acid sequence set forth in SEQ ID NO: 40.

In any of the above embodiments, the integrin binder binds human αvβ1, αvβ3, αvβ5, αvβ6, and αvβ8 integrins and mouse αvβ1, αvβ3, αvβ5, αvβ6, and αvβ8 integrins. Binding of the integrin binder may be determined using a cell-based assay, for example, the CELISA assay disclosed in the General Methods herein.

In a further embodiment, the integrin binder comprises an antibody comprising a heavy chain constant domain of the IgG1, IgG2, IgG3, or IgG4 isotype and a light chain constant domain of the human kappa or human lambda isotype.

In a further embodiment, the integrin binder comprises an antibody comprising a heavy chain constant domain of the IgG1 or IgG4 isotype and a light chain constant domain of the human kappa or human lambda isotype.

In a further embodiment, the integrin binder comprises an antibody having a heavy chain constant domain comprising an amino acid sequence having 90% sequence identity to the amino acid sequence set forth in SEQ ID NO: 41.

In a further embodiment, the heavy chain constant domain comprises the amino acid sequence set forth in SEQ ID NO: 41.

In a further embodiment, the light chain constant domain comprises an amino acid sequence comprising 90% identity to the amino acid sequence set forth in SEQ ID NO: 50.

In a further embodiment, the heavy chain constant domain of the IgG1 isotype comprises an Fc domain comprising one or more mutations that render the constant domain effector-silent.

In a further embodiment, the effector-silent constant domain comprises an amino acid sequence set forth in SEQ ID NO: 42, SEQ ID NO: 43, SEQ ID NO: 44, SEQ ID NO: 45, SEQ ID NO: 46, SEQ ID NO: 47, SEQ ID NO: 48, or SEQ ID NO: 49.

In a further embodiment, the light chain constant domain comprises an amino acid sequence comprising 90% identity to the amino acid sequence set forth in SEQ ID NO: 50.

In a further embodiment, the light chain constant domain comprises the amino acid sequence set forth in SEQ ID NO: 50.

In a further embodiment, the integrin binder comprises an antigen-binding fragment of an antibody selected from the group consisting of a Fab fragment, a Fab′ fragment, a F(ab′)₂ fragment, an Fv region, and an ScFv. In a further embodiment, the integrin binder is an antigen-binding fragment of an antibody selected from the group consisting of a Fab fragment, a Fab′ fragment, a F(ab′)₂ fragment, an Fv region, and an ScFv.

In a further embodiment, the integrin binder comprises an ScFv or Fab. In a further embodiment, the integrin binder is an ScFv or Fab.

The present invention further provides a composition comprising any one of the aforementioned integrin binders and a pharmaceutically acceptable carrier or diluent.

The present invention further provides a method for treating cancer or fibrosis in an individual in need thereof comprising administering to the individual a therapeutically effective amount of any one of the integrin binders disclosed herein or a composition disclosed herein to treat the cancer or fibrosis.

The present invention further provides any one of the integrin binders disclosed herein or a composition disclosed herein for treatment of cancer or fibrosis.

The present invention further provides for the use of any one of the integrin binders disclosed herein or a composition disclosed herein for the manufacture of a medicament for treating cancer or fibrosis.

The present invention further provides a combination therapy for treating cancer or fibrosis comprising any one of the integrin binders disclosed herein or a composition disclosed herein and a therapeutic agent. In a further embodiment, the therapeutic agent is a chemotherapy agent or a therapeutic antibody.

In particular embodiments of the methods, uses and compositions herein, the fibrosis is idiopathic pulmonary fibrosis.

The present invention further provides a nucleic acid molecule encoding any one of the integrin binders disclosed herein. In a further embodiment, the present invention provides an expression vector comprising one or more of the nucleic acid molecules disclosed herein. The present invention further provides a host cell comprising the expression vector disclosed herein. The present invention further provides a method for producing an integrin binder disclosed herein comprising (a) providing a host cell disclosed herein; (b) cultivating the host cell in a medium under conditions suitable for expressing the integrin binder; and (c) isolating the integrin binder from the medium.

The present invention further provides any one of the integrin binders disclosed herein conjugated to a detectable moiety. In a further embodiment, the detectable moiety is detectable by magnetic resonance imaging (MRI) or by X-ray imaging.

The present invention further provides a method for detecting integrin expression on the surface of cells in an individual comprising administering to the individual any one of the integrin binders disclosed herein conjugated to a detectable moiety and detecting the cells in the individual bound to the integrin binder.

The present invention further provides a method for treating idiopathic pulmonary fibrosis in an individual in need of the treatment comprising administering to the individual a therapeutically effective amount of (3S)-3-(6-methoxypyridin-3-yl)-3-[2-oxo-3-[3-(5,6,7,8-tetrahydro-1,8-naphthyridin-2-yl)propyl]imidazolidin-1-yl]propanoic acid to treat the idiopathic pulmonary fibrosis.

The present invention further provides (3S)-3-(6-methoxypyridin-3-yl)-3-[2-oxo-3-[3-(5,6,7,8-tetrahydro-1,8-naphthyridin-2-yl)propyl]imidazolidin-1-yl]propanoic acid for treatment of idiopathic pulmonary fibrosis.

The present invention further provides for the use of (3S)-3-(6-methoxypyridin-3-yl)-3-[2-oxo-3-[3-(5,6,7,8-tetrahydro-1,8-naphthyridin-2-yl)propyl]imidazolidin-1-yl]propanoic acid for the manufacture of a medicament for treating idiopathic pulmonary fibrosis.

### BRIEF DESCRIPTION OF THE DRAWINGS

FIG. 1A-1C. Changes of integrin expression upon fibrosis induction in the lung. (FIG. 1A) The expression of integrins in various human primary lung cell types upon TGFβ (5 ng/mL for 24 hours) treatment. Following immunoprecipitation with an anti-av antibody, the αvβ1, αvβ3, αvβ5, and αvβ6 heterodimers were detected by SALLY SUE simple western analysis after using antibodies that recognize each individual β-subunit. Normal human lung fibroblast, NHLF; normal human bronchial epithelial cells, NHBE; small airway epithelial cells, SAEC; bronchial smooth muscle cells, BSMC; pulmonary artery smooth muscle cells, PASMC; pulmonary artery endothelial cells, PAEC. L230 is an anti-av monoclonal antibody available from Enzo Life Sciences, which was used herein as a control. Full-length blot images are presented in FIG. 12A-12E. (FIG. 1B) Development of a bleomycin-induced lung fibrosis model in mice. Bleomycin (BLM) was administered at the indicated doses via intra-tracheal (i.t.) instillation. After 20 days, lungs were collected for histological analyses. Modified Ashcroft score, Picosirus red staining, immunohistochemical analyses of alpha smooth muscle actin (αSMA) and CD68 of total lung were quantified and shown (mean±SEM, n=5). Immunohistochemistry, IHC. One-way ANOVA followed by Tukey's test, *p<0.05, **p<0.01, ***p<0.005 vs Saline group. (FIG. 1C) Integrin expression and signaling in fibrotic lungs (BLM 0.5 U/kg body weight) was determined by SALLY SUE simple western analysis using antibodies that recognized the individual a or β-subunits. Each lane represents total lung homogenate for one animal, n=5 for saline or BLM group. Glyceraldehyde 3-phosphate dehydrogenase (GAPDH) level in total lung lysates was used as a loading control. Full-length blot images are presented in Supplemental FIG. 12F-12N.

FIG. 2A-2G. MK-0429 inhibits lung fibrosis in the bleomycin mouse model. (FIG. 2A) Schematics of compound administration in BLM model. 5 days after BLM intra-tracheal instillation, the animals were given MK-0429 (200 mpk via osmotic minipump for two weeks) or Nintedanib (60 mpk po qd for two weeks). Lungs were collected at Day 19 for histological and biochemical evaluation. mpk, milligrams per kilogram body weight; po, Peros, oral administration; qd, Quaque die, every day. (FIG. 2B) Plasma total drug concentration was measured 2 hours after final oral dose at Day 19. (FIG. 2C) Representative Masson Trichrome staining of mouse lungs. a and d, saline intra-tracheal instillation; b-f, BLM intra-tracheal instillation: a and d, no compound treatment; b, vehicle in minipump; c, MK-0429 in minipump; e, vehicle po; f, Nintedanib po. (FIG. 2D) Modified Ashcroft scores of mouse lung. Mean±SEM, n=10. One-way ANOVA followed by Tukey's test, *p<0.05, **p<0.01, ***p<0.005 vs BLM-vehicle group. (FIG. 2E) Total inflammation area in mouse lungs. Mean±SEM, n=10. One-way ANOVA followed by Tukey's test, *p<0.05, **p<0.01, ***p<0.005 vs BLM-vehicle group. (FIG. 2F) Immunohistochemical analysis of αSMA. Mean±SEM, n=10. One-way ANOVA followed by Tukey's test, *p<0.05, **p<0.01, ***p<0.005 vs BLM-vehicle group. (FIG. 2G) Soluble collagen content in bronchoalveolar lavage fluid (BALF). Mean±SEM, n=10. *p<0.05, **p<0.01, ***p<0.005 vs BLM-vehicle group.

FIG. 3A-3D. Integrin antibody screening and assay development. (FIG. 3A) Staged efforts to screen integrin antibodies from human naïve IgG library using a yeast display platform. (FIG. 3B) IgG-expressing yeast clone selection process. The X-axis represents integrin binding and the Y-axis reflects antibody expression. Yeast cell population with strong antigen binding (boxed) was sorted out for the next round of selection. (FIG. 3C) Cell-based ELISA (CELISA) binding assays were used as the primary screen for integrin antibody selection. Dose-dependent binding of control antibody abituzumab (mAb-24), which binds to various integrin-expressing CHOK1 cells was shown. (FIG. 3D) AlphaLISA integrin-ligand binding assays were used for in vitro functional screen. Dose-dependent inhibition of human integrin-ligand binding by MK-0429 in AlphaLISA assay panel.

FIG. 4. Discovery a set of antibodies with strong blocking activities against both human and mouse integrins. Titration of Ab-29, Ab-30, Ab-31, Ab-32, and Ab-33 for their binding to CHOK1-human αvβ1, αvβ6, and α5β1 stable cell lines in CELISA assays.

FIG. 5A-5B. Ab-31 inhibits integrin-mediated cell adhesion and latent TGFβ activation. (FIG. 5A) The effect of Ab-31 on the adhesion of CHOK1 parental, CHOK1-α5KO-mαvβ1, CHOK1-mαvβ3, and CHOK1-mαvβ5 cells to fibronectin or vitronectin matrix. (FIG. 5B) pan-integrin inhibitors suppress latent TGFβ activation in the transfected Mink lung epithelial cells (TMLC) and CHOK1-integrin co-culture system. The effects of Ab-31, MK-0429, and mAb-24 on PAI-1 luciferase activity were shown.

FIG. 6A-6C. Ab-31 reduces TGFβ-induced uSMA expression in lung fibroblasts. (FIG. 6A) Normal human lung fibroblasts were stimulated with TGFβ (5 ng/mL) and stained with anti-αSMA antibody for immunofluorescence analysis. Cells were pre-treated with or without MK-0429 (10 M) or the ALK5 inhibitor SB-525334 (10 μM) 30 minutes before the addition of TGFβ. 48 hours after the treatment, cells were imaged with Opera Phenix high-content screening system for αSMA expression (fluorescence intensity) and αSMA-associated morphological changes (STAR program). (FIG. 6B) The effects of Ab-31, MK-0429, and mAb-24 on TGFβ-associated αSMA induction in IPF patient lung fibroblasts. (FIG. 6C) Structural modeling predicts a distinct integrin binding mode for Ab-31. The structures of two therapeutic monoclonal antibodies, Abituzumab (17E6, RCSB PDB: 4002) and LM609 (RCSB PDB: 6AVQ), in complex with αvβ3 integrin were shown to highlight the difference in binding modes for each molecule. A model of Ab-31 and αvβ3 integrin complex was determined by docking of related antibody sequence to αvβ3 structure (see “General Methods”). For visualization, only the Fv region of each antibody were shown.

FIG. 7A-7E. Histology analysis of bleomycin-induced lung fibrosis model. (FIG. 7A) Representative histological images of mouse lungs from each experimental group. Scale bar, 100 μM. Immunohistochemistry, IHC. Quantitative analysis of each lung lobe, (FIG. 7B) Modified Ashcroft score, (FIG. 7C) αSMA positive area, (FIG. 7D) Area of Picosirus red (PSR) positive staining, and (FIG. 7E) Percentage of CD68-positive cells. Mean±SEM, n=5. One-way ANOVA followed by Tukey's test, *p<0.05, **p<0.01, ***p<0.005 vs Saline group.

FIG. 8A-8C. MK-0429 inhibits lung fibrosis in mouse bleomycin model. (FIG. 8A) Body weight of each experimental group over the duration of time course. Mean±SEM, n=10. One-way ANOVA followed by Tukey's test, **p<0.01, ***p<0.001 vs Saline group; ##p<0.01, ###p<0.001 vs BLM-vehicle group. (FIG. 8B) Percentage of body weight changes in each experimental group. Mean±SEM, n=10. One-way ANOVA followed by Tukey's test, **p<0.01, ***p<0.001 vs Saline group; ##p<0.01, ###p<0.001 vs BLM-vehicle group. (FIG. 8C) TIMP1 concentration in BALFs. Mean±SEM, n=10. One-way ANOVA followed by Tukey's test, *p<0.05, **p<0.01, ***p<0.001 vs BLM-vehicle group.

FIG. 9A-9E. The expression of integrins in CHOK1 stable lines. (FIG. 9A) FACS of mouse αv and pl expression in CHOK1-α5KO-mαvβ1 cells. Anti-av (RMV7) antibody and anti-β1 (KMI6) antibody were used for detection. (FIG. 9B) FACS of mouse αv and Q3 expression in CHOK1-mαvβ3 cells. Anti-av (RMV7) antibody and anti-03 (HM03.1) antibody were used for detection. (FIG. 9C) FACS of mouse αv and β5 expression in CHOK1-mav$5 cells. Anti-av (RMV7) antibody and anti-05 (P1F6) antibody were used for detection. (FIG. 9D) FACS of mouse αvβ6 expression in CHOK1-mavD6 cells. Anti-αvβ6 (10D5) antibody was used for detection. (FIG. 9E) FACS of mouse αv and β8 expression in CHOK1-mavD8 cells. Anti-av (RMV7) antibody and anti-08 antibody (ADWA-11; Stockis et al., Proc. Natl. Acad. Sci. (USA) 114: E10161-E10168 (2017)) were used for detection.

FIG. 10A-10B. Integrin antibodies with strong blocking activities against both human and mouse integrins. (FIG. 10A) Titration of Ab-29, Ab-30, Ab-31, Ab-32, and Ab-33 for their binding to CHOK1-mouse αvβ1, αvβ6, and α5β1 stable cell lines in CELISA assays. (FIG. 10B) Dose-dependent inhibition of mouse integrin ligand binding by MK-0429 in AlphaLISA assay panel.

FIG. 11. MK-0429 inhibits integrin-mediated cell adhesion. The effect of MK-0429 on the adhesion of CHOK1 parental, CHOK1-α5KO-mαvβ1, CHOK1-mαvβ3, and CHOK1-mαvβ5 cells to fibronectin or vitronectin matrix.

FIG. 12A-12E. SALLY SUE simple western full-length blot images for the blot images presented in in FIG. 1A. The expression of various integrins in human primary lung cell types upon TGFβ(5 ng/ml for 24 hours) treatment. Following immunoprecipitation with an anti-αv antibody, the αvβ1, αvβ3, αvβ5, and αvβ6 heterodimers were detected by SALLY SUE simple western analysis after using antibodies that recognize each individual P-subunit. Normal human lung fibroblast, NHLF; normal human bronchial epithelial cells, NHBE; small airway epithelial cells, SAEC; bronchial smooth muscle cells, BSMC; pulmonary artery smooth muscle cells, PASMC; human pulmonary artery endothelial cells, HPAEC; Hematopoietic stem cells, HSC; human primary kidney fibroblast, HPKF; Renal Proximal Tubule Epithelial Cells, RPTEC; Podocytes, epithelial cells of Bowman's capsule in the kidneys that wrap around capillaries of the glomerulus; Normal Human Mesangial Cells, NHMC. Integrin antibodies were reported in Table 8. Exposure time: 4 seconds.

FIG. 12F-12N. SALLY SUE simple western full-length blot images for blot images presented in FIG. 1C. Integrin expression and signaling in fibrotic lungs was determined by SALLY SUE simple western analysis using antibodies that recognized the individual subunits. GAPDH level in total lung lysates was used as a loading control. Integrin antibodies were reported in Table 8. Exposure time: 4 seconds.

### DETAILED DESCRIPTION OF THE INVENTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule λ for its partner Y can generally be represented by the dissociation constant (KD). Affinity can be measured by common methods known in the art, including KinExA and surface plasmon resonance (SPR; Biacore™). Specific illustrative and exemplary embodiments for measuring binding affinity are described in the following.

As used herein, the term “administration” and “treatment,” as it applies to an animal, human, experimental subject, cell, tissue, organ, or biological fluid, refers to contact of an exogenous pharmaceutical, therapeutic, diagnostic agent, or composition comprising a human integrin binder as disclosed herein to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., human, rat, mouse, dog, cat. rabbit). In a preferred embodiment, the term “subject” refers to a human.

As used herein, the term “amino acid” refers to a simple organic compound containing both a carboxyl (—COOH) and an amino (—NH₂) group. Amino acids are the building blocks for proteins, polypeptides, and peptides. Amino acids occur in L-form and D-form, with the L-form in naturally occurring proteins, polypeptides, and peptides. Amino acids and their code names are set forth in the following chart.

| Amino acid | Three letter code | One letter code |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “antibody” or “immunoglobulin” as used herein refers to a glycoprotein comprising at least two heavy chains (HCs) and two light chains (LCs) inter-connected by disulfide bonds. Each HC is comprised of a heavy chain variable region or domain (V_{H}) and a heavy chain constant region or domain. Each light chain is comprised of an LC variable region or domain (V_{L}) and a LC constant domain. In certain naturally occurring IgG, IgD, and IgA antibodies, the heavy chain constant region is comprised of three domains, CH1, CH2 and CH3. In general, the basic antibody structural unit for antibodies is a Y-shaped tetramer comprising two HC/LC pairs (2H). Each tetramer includes two identical pairs of polypeptide chains, each pair having one LC (about 25 kDa) and HC chain (about 50-70 kDa) (H+L). Each HC:LC pair comprises one V_{H}: one V_{L} pair. The one V_{H}:one V_{L} pair may be referred to by the term “Fab”. Thus, each antibody tetramer comprises two Fabs, one per each arm of the Y-shaped antibody.

The LC constant domain is comprised of one domain, CL. The human V_{H} includes seven family members: V_{H}1, V_{H}2, V_{H}3, V_{H}4, V_{H}5, V_{H}6, and V_{H}7; and the human V_{L} includes 16 family members: V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10. Each of these family members can be further divided into particular subtypes. The V_{H} and V_{L} can be further subdivided into regions of hypervariability, termed complementarity determining region (CDR) areas, interspersed with regions that are more conserved, termed framework regions (FR). Each V_{H} and V_{L} is composed of three CDR regions and four FR regions, arranged from amino-terminus to carboxy-terminus in the following order: FR1, CDR 1, FR2, CDR 2, FR3, CDR 3, FR4. Numbering of the amino acids in a VH may be determined using the Kabat numbering scheme. See Béranger, et al., Ed. Ginetoux, Correspondence between the IMGT unique numbering for C-DOMAIN, the IMGT exon numbering, the Eu and Kabat numberings: Human IGHG, Created: 17/05/2001, Version: 08/06/2016, which is accessible at www.imgt.org/IMGTScientificChart/Numbering/Hu_IGHGnber.html).

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG1 (Eu), which has a constant domain that begins at amino acid position 118 of the amino acid sequence of the IgG1 described in Edelman et al., Proc. Natl. Acad. Sci. USA. 63: 78-85 (1969), and is shown for the IgG1, IgG2, IgG3, and IgG4 constant domains in Béranger et al., op. cit.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following.
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence);
Chothia numbering scheme is based on the location of the structural loop region (See Chothia & Lesk, J. Mol. Biol. 196: 901-917 (1987); Al-Lazikani et al., J. Mol. Biol. 273: 927-948 (1997));
AbM numbering scheme is a compromise between the two used by Oxford Molecular's AbM antibody modelling software (see Karu et al, ILAR Journal 37: 132-141 (1995);
Contact numbering scheme is based on an analysis of the available complex crystal structures (See www.bioinf.org.uk: Prof. Andrew C. R. Martin's Group; Abhinandan & Martin, Mol. Immunol. 45:3832-3839 (2008)).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509(1997); Lefranc et al., Dev Comp Immunol. 27:55-77 (2003)).

The following general rules disclosed in www.bioinf.org.uk: Prof. Andrew C. R. Martin's Group and reproduced in Table 1 below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 1

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat Numbering)³ | | | | |
| H1 | H31--H35 | H26--H35 | H26--H32 | H30--H35 | H26--H33 |
| | (Chothia Numbering) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

The entire nucleotide sequence of the heavy chain and light chain variable regions are commonly numbered according to Kabat while the three CDRs within the variable region may be defined according to any one of the aforementioned numbering schemes.

In general, the state of the art recognizes that in many cases, the CDR 3 region of the heavy chain is the primary determinant of antibody specificity, and examples of specific antibody generation based on CDR 3 of the heavy chain alone are known in the art (e.g., Beiboer et al., J. Mol. Biol. 296: 833-849 (2000); Klimka et al., British J. Cancer 83: 252-260 (2000); Rader et al., Proc. Natl. Acad. Sci. USA 95: 8910-8915 (1998); Xu et al., Immunity 13: 37-45 (2000).

As used herein, the term “Fc domain”, or “Fc” as used herein is the crystallizable fragment domain or region obtained from an antibody that comprises the CH2 and CH3 domains of an antibody. In an antibody, the two Fc domains are held together by two or more disulfide bonds and by hydrophobic interactions of the CH3 domains. The Fc domain may be obtained by digesting an antibody with the protease papain. Typically, amino acids in the Fc domain are numbered according to the Eu numbering convention (See Edelmann et al., Biochem. 63: 78-85 (1969)).

As used herein, the term “antigen” as used herein refers to any foreign substance which induces an immune response in the body.

As used herein, the term “antigen binding fragment” refers to a polypeptide or polypeptides comprising a fragment of a full-length antibody, which retains the ability to specifically bind to the antigen bound by the full-length antibody, and/or to compete with the full-length antibody for specifically binding to the antigen. Examples of antigen binding fragments include but are not limited to Fab fragment, Fab′ fragment, F(ab′)2 fragment, Fv region, and scFv.

As used herein, “specifically binds” refers, with respect to a target antigen, to the preferential association of a binder, in whole or part, with the target antigen and not to other molecules, particularly molecules found in human blood or serum. Binders as shown herein typically bind specifically to the target antigen with high affinity, reflected by a dissociation constant (K_{D}) of 10-7 to 10⁻¹¹ M or less. Any K_{D} greater than about 10⁻⁶ M is generally considered to indicate nonspecific binding. As used herein, a binder that “specifically binds” or “binds specifically” to a target antigen refers to a binder that binds to the target antigen with high affinity, which means having a K_{D} of 10⁻⁷ M or less, in particular embodiments a K_{D} of 10⁻⁸ M or less, or 5×10⁻⁹ M or less, or between 10⁻⁸ M and 10⁻¹¹ M or less, but does not bind with measurable binding to a non-target antigen as determined in a cell ELISA or Surface Plasmon Resonance assay (SPR; Biacore) using 10 μg/mL antibody. Particular embodiments of the present invention are any one of the integrin binders disclosed herein having a binding affinity to human αvβ1, αvβ3, αvβ5, αvβ6, αvβ8, and αv 5β1 integrins and mouse αvβ1, αvβ3, αvβ5, αvβ6, and αvβ8 integrins of K_{D} of 10⁻⁷ M or less, in particular embodiments a K_{D} of 10⁻⁸ M or less, or 5×10⁻⁹ M or less, or between 10⁻⁸ M and 10⁻¹¹ M or less.

As used herein, the term “Fab fragment” refers to an antigen binder comprising one antibody light chain and the CH1 and V_{H} of one antibody heavy chain. The heavy chain of a Fab molecule cannot form a disulfide bond with another heavy chain molecule. A “Fab fragment” can be the product of papain cleavage of an antibody.

As used herein, the term “Fab′ fragment” refers to an antigen binder comprising one antibody light chain and a portion or fragment of one antibody heavy chain that contains the V_{H} and the CH1 domain up to a region between the CH1 and CH2 domains, such that an interchain disulfide bond can be formed between the two heavy chains of two Fab′ fragments to form a F(ab′)₂ molecule.

As used herein, the term “F(ab′)2 fragment” refers to an antigen binder comprising two antibody light chains and two heavy chains containing the V_{H} and the CH1 domain up to a region between the CH1 and CH2 domains, such that an interchain disulfide bond is formed between the two heavy chains. An F(ab′)₂ fragment thus is composed of two Fab′ fragments that are held together by a disulfide bond between the two heavy chains. An “F(ab′)₂ fragment” can be the product of pepsin cleavage of an antibody.

As used herein, the term “Fv region” refers to an antigen binder comprising the variable regions from both the heavy and light chains of an antibody but lacks the constant regions.

As used herein, the term “ScFv” or “single-chain variable fragment” refers to a fusion protein comprising a V_{H} and V_{L} fused or linked together by a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker.

As used herein, the term “diabody” refers to an antigen binder comprising a small antibody fragment with two antigen-binding regions, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementarity domains of another chain and create two antigen-binding regions. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

These and other potential constructs are described at Chan & Carter (2010) Nat. Rev. Immunol. 10:301. These antibody fragments are obtained using conventional techniques known to those with skill in the art, and the fragments are screened for utility in the same manner as are intact antibodies. Antigen-binding fragments can be produced by recombinant DNA techniques, or by enzymatic or chemical cleavage of intact immunoglobulins.

As used herein, the term “chimeric antigen receptor” (CAR) refers to a recombinant polypeptide comprising at least an extracellular domain that binds specifically to an antigen or a target, a transmembrane domain and an intracellular T cell receptor-activating signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

As used herein, the term “extracellular antigen binding domain,” “extracellular domain,” or “extracellular ligand binding domain” when used in reference to a CAR refers to the part of a CAR that is located outside of the cell membrane and is capable of binding to an antigen, target or ligand.

As used herein, the term “hinge region” when used in reference to a CAR refers to the part of a CAR that connects two adjacent domains of the CAR protein, e.g., the extracellular domain and the transmembrane domain.

As used herein, the term “transmembrane domain” refers to the portion of a CAR that extends across the cell membrane and anchors the CAR to cell membrane.

As used herein, the term “intracellular T cell receptor-activating signaling domain”, “cytoplasmic signaling domain,” or “intracellular signaling domain” refers to the part of a CAR that is located inside of the cell membrane and is capable of transducing an effector signal.

As used herein, the term “isolated” antibodies or antigen-binding fragments thereof are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

As used herein, the term “monoclonal antibody” refers to a population of substantially homogeneous antibodies, i.e., the antibody molecules comprising the population are identical in amino acid sequence except for possible naturally occurring mutations that may be present in minor amounts. In contrast, conventional (poly clonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al., Nature 256: 495 (1975) or may be made by recombinant DNA methods (see, e.g., U.S. Pat. No. 4,816,567). The “monoclonal antibodies” may also be isolated from phage antibody libraries using the techniques described in Clackson et al., Nature 352: 624-628 (1991), and Marks et al., J. Mol. Biol. 222: 581-597 (1991). for example. See also Presta, J. Allergy Clin. Immunol. 116: 731 (2005).

As used herein, the term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters. Genes include both naturally occurring nucleotide sequences encoding a molecule of interest and synthetically derived nucleotide sequences encoding a molecule of interest, for example, complementary DNA (cDNA) obtained from a messenger RNA (mRNA) nucleotide sequence.

As used herein, the term “germline” or “germline sequence” refers to a sequence of unrearranged immunoglobulin DNA sequences. Any suitable source of unrearranged immunoglobulin sequences may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al., Nucleic Acids Res. 33: D256-D261 (2005).

As used herein, the term “library” as used herein is, typically, a collection of related but diverse polynucleotides that are, in general, in a common vector backbone. For example, a light chain or heavy chain immunoglobulin library may contain polynucleotides, in a common vector backbone, that encode light and/or heavy chain immunoglobulins, which are diverse but related in their nucleotide sequence; for example, which immunoglobulins are functionally diverse in their abilities to form complexes with other immunoglobulins, e.g., in an antibody display system of the present invention, and bind a particular antigen.

As used herein, the term “polynucleotides” discussed herein form part of the present invention. A “polynucleotide”, “nucleic acid” or “nucleic acid molecule” include DNA and RNA, single- or double-stranded. Polynucleotides e.g., encoding an immunoglobulin chain or component of the antibody display system of the present invention, may, in an embodiment of the invention, be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

Polynucleotides e.g., encoding an immunoglobulin chain or component of the antibody display system of the present invention, may be operably associated with a promoter. A “promoter” or “promoter sequence” is, in an embodiment of the invention, a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease Si), as well as protein binding domains (consensus sequences) responsible for the binding of RNA polymerase. The promoter may be operably associated with other expression control sequences, including enhancer and repressor sequences or with a nucleic acid of the invention. Promoters which may be used to control gene expression include, but are not limited to, cytomegalovirus (CMV) promoter (U.S. Pat. Nos. 5,385,839 and 5,168,062), the SV40 early promoter region (Benoist, et al., Nature 290: 304-310 (1981)), the promoter contained in the 3′ long terminal repeat of Rous sarcoma virus (Yamamoto et al., Cell 22: 787-797 (1980)), the herpes thymidine kinase promoter (Wagner et al., Proc. Natl. Acad. Sci. USA 78: 1441-1445 (1981)), the regulatory sequences of the metallothionein gene (Brinster et al., Nature 296: 39-42 (1982)); prokaryotic expression vectors such as the P-lactamase promoter (Villa-Komaroff et al., Proc. Natl. Acad. Sci. USA 75: 3727-3731 (1978)), or the tac promoter (DeBoer et al., Proc. Natl. Acad. Sci. USA 80: 21-25 (1983)); see also “Useful proteins from recombinant bacteria” in Scientific American 242: 74-94 (1980); and promoter elements from yeast or other fungi such as the Gal 4 promoter, the ADC (alcohol dehydrogenase) promoter, PGK (phosphoglycerol kinase) promoter or the alkaline phosphatase promoter.

As used herein, the terms “vector”, “cloning vector” and “expression vector” include a vehicle (e.g., a plasmid) by which a DNA or RNA sequence can be introduced into a host cell so as to transform the host and, optionally, promote expression and/or replication of the introduced sequence. Polynucleotides encoding an immunoglobulin chain or component of the antibody display system of the present invention may, in an embodiment of the invention, be in a vector.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny of a parent cell will have precisely identical DNA content, due to deliberate or inadvertent mutations. Mutant progeny having the same function or biological activity as screened for in the originally transformed cell are included. Where distinct designations are intended, it will be clear from the context.

As used herein, the term “control sequences” or “regulatory sequences” refers to DNA sequences necessary for the expression of an operably linked coding sequence in a particular host organism. The control sequences that are suitable for expression in eukaryotes, for example, include a promoter, operator or enhancer sequences, transcription termination sequences, and polyadenylation sequences for expression of a messenger RNA encoding a protein and a ribosome binding site for facilitating translation of the messenger RNA.

As used herein, a nucleic acid is “operably linked” when it is placed into a functional relationship with another nucleic acid sequence, e.g., a regulatory sequence. For example, DNA for a pre-sequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “expression” as used herein is defined as the transcription and/or translation of a particular nucleotide sequence.

As used herein, the term “treat” or “treating” means to administer a therapeutic agent, such as a composition containing any of the human integrin binders of the present invention, topically, subcutaneously, intramuscular, intradermally, or systemically to an individual in need. The amount of a therapeutic agent that is effective to treat cancer or proliferative disease in the individual may vary according to factors such as the injury or disease state, age, and/or weight of the individual, and the ability of the therapeutic agent to elicit a desired response in the individual. Whether the therapeutic objective has been achieved can be assessed by the individual and/or any clinical measurement typically used by physicians or other skilled healthcare providers to assess the severity or progression status of the treatment. Thus, the terms denote that a beneficial result has been or will be conferred on a human or animal individual in need.

As used herein, the term “treatment,” as it applies to a human or veterinary individual, refers to therapeutic treatment, as well as diagnostic applications. “Treatment” as it applies to a human or veterinary individual, encompasses contact of the antibodies or antigen binding fragments of the present invention to a human or animal subject.

As used herein, the term “therapeutically effective amount” refers to a quantity of a specific substance sufficient to achieve a desired effect in an individual being treated. For instance, this may be the amount necessary to inhibit or reduce the severity of a disease or disorder in an individual.

As used herein, the term “combination therapy” refers to treatment of a human or animal individual comprising administering a first therapeutic agent and a second therapeutic agent consecutively or concurrently to the individual. In general, the first and second therapeutic agents are administered to the individual separately and not as a mixture; however, there may be embodiments where the first and second therapeutic agents are mixed prior to administration. As used herein, the term “MK-0429” refers to (3S)-3-(6-methoxypyridin-3-yl)-3-[2-oxo-3-[3-(5,6,7,8-tetrahydro-1,8-naphthyridin-2-yl)propyl]imidazolidin-1-yl]propanoic acid having the formula

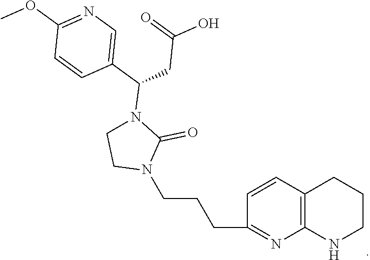 [SMILES: COc1ccc([C@H](CC(=O)O)N2CCN(CCCc3ccc4c(n3)NCCC4)C2=O)cn1]

MK-0429 has been previously disclosed in U.S. Pat. No. 6,017,926; U.S. Pat. Pubs. US20020040039 and US20040053968; and in Hutchinson et al., J. Med. Chem. 46(22):4790-8 (2003); Pickarski et al., Oncol. Rep. 33(6):2737-45 (2015); Zhou et al., Pharmacol. Res. Perspect. 5(5):e00354 (2017); Zhang et al., Scientific Reports 11: 2118 (2021, Pub online Jan. 22, 2021); Zhang et al., bioRxiv preprint https://doi.org/10.1101/2020.07.20.207555 (pub online Jul. 22, 2020).

### Introduction

Lung fibrosis, or the scarring of the lung, is a devastating disease with huge unmet medical need. There are limited treatment options and its prognosis is worse than most types of cancer. We previously discovered that MK-0429 is an equipotent pan-inhibitor of integrins that reduces proteinuria and kidney fibrosis in a preclinical model. In search of newer integrin inhibitors for fibrosis, we characterized fully-human monoclonal antibodies discovered using a yeast display platform. We identified several potent neutralizing anti-integrin antibodies with unique human and mouse cross-reactivity. Among these, several blocked the binding of multiple integrins to their ligands with IC₅₀'s comparable to those of MK-0429. Furthermore, both MK-0429 and several of the antibodies suppressed integrin-mediated cell adhesion and latent TGFβ activation. In IPF patient lung fibroblasts, TGFβ treatment induced profound αSMA expression in phenotypic imaging assays and these antibodies demonstrated potent in vitro activity at inhibiting αSMA expression, suggesting that the anti-integrin antibody is able to modulate TGFβ action though mechanisms beyond the inhibition of latent TGFβ activation.

IPF is multi-factorial disease and the dominant mechanism that drives pathogenesis is unclear. The mechanism of action for Pirfenidone is presently unknown, likely involving multiple pathways that include anti-inflammation and TGFβ suppression (Takeda et al, Patient Prefer. Adherence 8: 361-370 (2014)). Nintedanib is an inhibitor for multiple receptor tyrosine kinases, such as VEGFR, PDGFR, and FGFR (Wollin et al., Eur. Respir. J. 45: 1434-1445 (2015)). Currently, there are several mechanisms being tested in the clinic for IPF patients, including but not limited to the CTGF antibody Pamrevlumab, the Autotaxin inhibitor GLPG-1690, and recombinant Pentraxin 2 (PRM-151).

In recent years, integrin inhibitors have emerged as key mediators of tissue fibrosis. In particular, av-containing integrins, such as αvβ6, modulate local TGFβ activation and myofibroblast activation with strong preclinical validation for lung fibrosis (Munger et al., Cell 96, 319-328 (1999); Horan et al., Am. J. Respir. Crit. Care. Med. 177, 56-65 (2008); Henderson & Sheppard, Biochim. Biophys. Acta 1832, 891-896, (2013)). We initiated an antibody discovery campaign and discovered a set of novel fully human anti-integrin monoclonal antibodies with human and mouse cross-reactivity. Among these, Ab-31 potently blocked integrin-ligand binding, inhibited integrin-mediated cell adhesion, suppressed both the activation of latent TGFβ and the αSMA expression induced by activated TGFβ. Notably, Ab-31 demonstrated potent activity at inhibiting TGFβ response in IPF patient lung fibroblasts. It is intriguing that both Ab-31 and MK-0429 have comparable activities when tested in vitro using AlphaLISA integrin-ligand blocking assays while their respective impacts on TGFβ signaling in IPF patient lung fibroblasts was drastically different. In previous reports, bivalent 17E6 and LM609 were postulated to interfere with integrin clustering and internalization on the cell surface, enhancing their therapeutic effects over that of a monovalent Fab fragment (Mahalingam et al., J. Biol. Chem. 289, 13801-13809 (2014); Borst et al., Structure 25, 1732-1739 (2017)).

The complexity of integrin biology and the overlapping roles of multiple integrins in the progression of the disease suggest that a pharmacological pan inhibitor would be beneficial, leading to clinically meaningful inhibition of fibrosis. Interestingly, a recent genome-wide association study (GWAS) study of large population provides strong genetic evidence that supports targeting the integrin to improve lung function, potentially expanding the use of integrin inhibitors to a broader patient population, such as chronic obstructive pulmonary disease (COPD). The poly-pharmacology nature of an αv inhibitor raises potential safety concerns. Notably, MK-0429 has been tested in the clinic in osteoporosis patients over the duration of 52 weeks with relatively well-tolerated safety profile (Murphy et al., J. Clin. Endocrinol. Metab. 90, 2022-2028 (2005); Rosenthal et al., Asia Pac. J. Clin. Oncol. 6, 42-48 (2010)).

One main mechanism-of-action of integrin inhibitors is to inhibit latent TGFβ activation. Compared to the preclinical cardiovascular safety signal observed with TGFβ receptor inhibition, the safety profile of pan-integrin inhibition is more tolerable. Recently, an αvβ6 antibody (BG00011) was withdrawn from phase 2 clinical trials in IPF patients due to safety concerns (clinicaltrials.gov identifier NCT03573505). The pan-av integrin binders of the present invention provide an improvement over currently available integrin inhibitors.

We also developed a small molecule integrin inhibitor MK-0429 with good oral bioavailability in humans (Murphy et al., J. Clin. Endocrinol. Metab. 90, 2022-2028 (2005)). MK-0429 was initially designed as an RGD mimetic against αvβ3 integrin that incorporates key pharmacophores representing the guanidine and carboxylic acid of the RGD tripeptide sequence (Hutchinson et al., J. Med. Chem. 46, 4790-4798 (2003); Coleman et al., J. Med. Chem. 47, 4829-4837 (2004)). We recently found that MK-0429 is an equipotent pan-inhibitor of multiple av integrins, and it reduces proteinuria and renal fibrosis in an experimental diabetic nephropathy model (Zhou et al., Pharmacol. Res. Perspect. 5(5):e00354 (2017)). As disclosed herein, we further demonstrate that MK-0429 significantly inhibits fibrosis progression in a bleomycin-induced lung fibrosis mouse model.

### Integrin Binders

The integrin binders of the present invention are pan-av integrin binders comprising chimeric or fully human antibodies or antigen binding fragments thereof that specifically bind human integrins αvβ1, αvβ3, αvβ5, αvβ6, and αvβ8 integrins and mouse integrins αvβ1, αvβ3, αvβ5, αvβ6, and αvβ8 integrins as determined in the cell-based binding assay (CELISA) as disclosed in the General Methods herein. Exemplary integrin binders include antibodies Ab-29, Ab-30, Ab-31, Ab-32, and Ab-33. Antibodies Ab-29, Ab-30, Ab-31, and Ab-32 also bind human integrin αvβ1 as determined in the cell-based binding assay (CELISA) as disclosed in the General Methods herein.

The integrin binders disclosed herein comprise a V_{H} domain and a V_{L} domain, each domain comprising three CDRs and four Frameworks (FR) in the following arrangement
FR 1-CDR 1-FR 2-CDR 2-FR 3-CDR 3-FR 4.

These integrin binders comprise six complementarity determining regions (CDRs) comprising a particular combination of three CDRs from a V_{H} and three CDRs from the V_{L} that pairs with the V_{H}. The CDR sequences may be defined according to any numbering scheme useful for defining CDR sequences including but not limited to the Kabat, Chothia, AbM, ImMunoGeneTics (IMGT), or Contact numbering scheme. Guidance for defining the CDR sequences may be found in the general rules disclosed in www.bioinf.org.uk: Prof Andrew C. R. Martin's Group and reproduced in Table 1.

In a particular embodiment, the CDRs are defined by Kabat or IMGT. The CDR amino acid sequences shown in Tables 2-6 are set forth according to the Kabat numbering scheme for identifying CDR amino acid sequences.

TABLE 2
Amino Acid Sequences of integrin binder
Ab-29 (V_{H} and V_{L} CDRs as defined by Kabat)

| | CDR 1 | CDR 2 | CDR 3 |
|---|---|---|---|
| V_{H} | SNSYYWG | SIYYSGST | VRRVAGRGDS |
| | (SEQ ID | YYNPSLKS | YYYYYGMDV |
| | NO: 1) | (SEQ ID | (SEQ ID |
| | | NO: 2) | NO: 3) |
| V_{L} | RASQGISRWLA | AASSLQS | QQAISHPWT |
| | (SEQ ID | (SEQ ID | (SEQ ID |
| | \NO: 4) | NO: 5) | NO: 6) |

TABLE 3
Amino Acid Sequences of integrin binder
Ab-30 (V_{H} and V_{L} CDRs as defined by Kabat)

| | CDR 1 | CDR 2 | CDR 3 |
|---|---|---|---|
| V_{H} | SGYYWG | SIYHSGNT | VPRYSRGDA |
| | (SEQ ID | YYNPSLKS | YYYYGMDV |
| | NO: 7) | (SEQ ID | (SEQ ID |
| | | NO: 8) | NO: 9) |
| V_{L} | RASQSI | KASSLES | QQVKIY |
| | SSWLA | (SEQ ID | PPYT |
| | (SEQ ID | NO: 11) | (SEQ ID |
| | NO: 10) | | NO: 12) |

TABLE 4
Amino Acid Sequences of integrin binder-
Ab-31 (V_{H} and V_{L} CDRs as defined by Kabat)

| | CDR 1 | CDR 2 | CDR 3 |
|---|---|---|---|
| V_{H} | SGYYWG | SIYHSGNT | VPRYSRGDA |
| | (SEQ ID | YYNPSLKS | YYYYGMDV |
| | NO: 13) | (SEQ ID | (SEQ ID |
| | | NO: 14) | NO: 15) |
| V_{L} | RASQSI | KASSLES | QQSGPYPWT |
| | GSWLA | (SEQ ID | (SEQ ID |
| | (SEQ ID | NO: 17) | NO: 18) |
| | NO: 16) | | |

TABLE 5
Amino Acid Sequences of integrin binder
Ab-32 (V_{H} and V_{L} CDRs as defined by Kabat)

| | CDR 1 | CDR 2 | CDR 3 |
|---|---|---|---|
| V_{H} | SGYYWG | SIYHSGNT | VPRYSRGDA |
| | (SEQ ID | YYNPSLKS | YYYYGMDV |
| | NO: 19) | (SEQ ID | (SEQ ID |
| | | NO: 20) | NO: 21) |
| V_{L} | RASQSVR | GASSRAT | QQAGLVFT |
| | SSYLA | (SEQ ID | (SEQ ID |
| | (SEQ ID | NO: 23) | NO: 24) |
| | NO: 22) | | |

TABLE 6
Amino Acid Sequences of integrin binder
Ab-33 (V_{H} and V_{L} CDRs as defined by Kabat)

| | CDR 1 | CDR 2 | CDR 3 |
|---|---|---|---|
| V_{H} | NYAIS | GIIPIFGTA | APRSARGDM |
| | (SEQ ID | NYAQKFQG | YYYYYGMDV |
| | NO: 25) | (SEQ ID | (SEQ ID |
| | | NO: 26) | NO: 27) |
| V_{L} | RASQSI | KASSLES | QQYGRYPWT |
| | GSWLA | (SEQ ID | (SEQ ID |
| | (SEQ ID | NO: 29) | NO: 30) |
| | NO: 28) | | |

A particular CDR amino acid sequence determined using any one of the schemes for identifying CDR amino acid sequences (See Table 1) have more or less amino acids than that of CDR amino acid sequences identified according to any other numbering scheme but the CDR amino acid sequences will overlap to some extent. Thus, the CDR amino acid sequences defined according to Kabat are not to be construed as limiting and any integrin binder in which the CDR amino acid sequences have been identified by another numbering scheme will fall within the scope of the integrin binders of the present invention provided the amino acid sequences for such integrin binders comprise the six CDR amino acid sequences as identified by Kabat. For all integrin binders disclosed herein unless indicated otherwise, the amino acids comprising the variable domains as a whole are numbered according to the Kabat numbering scheme independently of how the amino acids comprising the CDR are defined. The heavy chain constant domains are numbered according to the Eu numbering scheme.

In particular embodiments of the invention, the integrin binder comprises (a) a V_{H} domain comprising a CDR 1 comprising the amino acid sequence set forth in SEQ ID NO: 1, a CDR 2 comprising the amino acid sequence set forth in SEQ ID NO: 2, and a CDR 3 comprising the amino acid sequence set forth in SEQ ID NO: 3; and (b) a V_{L} domain comprising a CDR 1 comprising the amino acid sequence set forth in SEQ ID NO: 4, a CDR 2 comprising the amino acid sequence set forth in SEQ ID NO: 5, and a CDR 3 comprising the amino acid sequence set forth in SEQ ID NO: 6, wherein the CDR sequences are defined by the Kabat numbering scheme.

In particular embodiments of the invention, the integrin binder comprises (a) a V_{H} domain comprising a CDR 1 comprising the amino acid sequence set forth in SEQ ID NO: 7, a CDR 2 comprising the amino acid sequence set forth in SEQ ID NO: 8, and a CDR 3 comprising the amino acid sequence set forth in SEQ ID NO: 9; and (b) a V_{L} domain comprising a CDR 1 comprising the amino acid sequence set forth in SEQ ID NO: 10, a CDR 2 comprising the amino acid sequence set forth in SEQ ID NO: 11, and a CDR 3 comprising the amino acid sequence set forth in SEQ ID NO: 12, wherein the CDR sequences are defined by the Kabat numbering scheme.

In particular embodiments of the invention, the integrin binder comprises (a) a V_{H} domain comprising a CDR 1 comprising the amino acid sequence set forth in SEQ ID NO: 13, a CDR 2 comprising the amino acid sequence set forth in SEQ ID NO: 14, and a CDR 3 comprising the amino acid sequence set forth in SEQ ID NO: 15; and (b) a V_{L} domain comprising a CDR 1 comprising the amino acid sequence set forth in SEQ ID NO: 16, a CDR 2 comprising the amino acid sequence set forth in SEQ ID NO: 17, and a CDR 3 comprising the amino acid sequence set forth in SEQ ID NO: 18, wherein the CDR sequences are defined by the Kabat numbering scheme.

In particular embodiments of the invention, the integrin binder comprises (a) a V_{H} domain comprising a CDR 1 comprising the amino acid sequence set forth in SEQ ID NO: 19, a CDR 2 comprising the amino acid sequence set forth in SEQ ID NO: 20, and a CDR 3 comprising the amino acid sequence set forth in SEQ ID NO: 21; and (b) a V_{L} domain comprising a CDR 1 comprising the amino acid sequence set forth in SEQ ID NO: 22, a CDR 2 comprising the amino acid sequence set forth in SEQ ID NO: 23, and a CDR 3 comprising the amino acid sequence set forth in SEQ ID NO: 24, wherein the CDR sequences are defined by the Kabat numbering scheme.

In particular embodiments of the invention, the integrin binder comprises (a) a V_{H} domain comprising a CDR 1 comprising the amino acid sequence set forth in SEQ ID NO: 25, a CDR 2 comprising the amino acid sequence set forth in SEQ ID NO: 26, and a CDR 3 comprising the amino acid sequence set forth in SEQ ID NO: 27; and (b) a V_{L} domain comprising a CDR 1 comprising the amino acid sequence set forth in SEQ ID NO: 28, a CDR 2 comprising the amino acid sequence set forth in SEQ ID NO: 29, and a CDR 3 comprising the amino acid sequence set forth in SEQ ID NO: 30, wherein the CDR sequences are defined by the Kabat numbering scheme.

In further embodiments, the integrin binder comprises a V_{H} domain comprising the amino acid sequence set forth in SEQ ID NO: 31 and a V_{L} domain comprising the amino acid sequence set forth in SEQ ID NO: 32.

In further embodiments, the integrin binder comprises a V_{H} domain comprising the amino acid sequence set forth in SEQ ID NO: 33 and a V_{L} domain comprising the amino acid sequence set forth in SEQ ID NO: 34.

In further embodiments, the integrin binder comprises a V_{H} domain comprising the amino acid sequence set forth in SEQ ID NO: 35 and a V_{L} domain comprising the amino acid sequence set forth in SEQ ID NO: 36.

In further embodiments, the integrin binder comprises a V_{H} domain comprising the amino acid sequence set forth in SEQ ID NO: 37 and a V_{L} domain comprising the amino acid sequence set forth in SEQ ID NO: 38.

In further embodiments, the integrin binder comprises a V_{H} domain comprising the amino acid sequence set forth in SEQ ID NO: 39 and a V_{L} domain comprising the amino acid sequence set forth in SEQ ID NO: 40.

In further embodiments of the invention, the integrin binder is an antibody comprising a heavy chain (HC) constant domain of the IgG₁ isotype. In particular embodiments, the heavy chain constant domain comprises 1, 2, 3, 4, 5, 6, 7, 8, 9, or 10 amino acid substitutions, additions, deletions, or combinations thereof compared to the amino acid sequence of the native IgG1 isotype.

IgG₁ heavy chain constant domain comprising the amino acid sequence shown in SEQ ID NO: 41 or a variant thereof comprising 1, 2, 3, 4, 5, 6, 7, 8, 9, or 10 amino acid substitutions, additions, deletions, or combinations thereof.

In particular embodiments of the invention, the constant domains as disclosed herein may comprise a C-terminal lysine or lack either a C-terminal lysine or a C-terminal glycine-lysine dipeptide.

In any one of the embodiments disclosed herein, the light chain may comprise a human kappa light chain constant domain comprising SEQ ID NO: 50.

Integrin binders Comprising an Effector-silent Fc Domain Integrin binders of the present invention may be full-sized antibodies that comprise an HC constant domain or Fc domain thereof that has been modified such that the antibody displays no measurable binding to one or more FcRs or displays reduced binding to one or more FcRs compared to that of an unmodified antibody of the same IgG isotype. Such effector-silent antibodies may in further embodiments display no measurable binding to each of FcγRIIIa, FcγRIIa, and FcγRI or display reduced binding to each of FcγRIIIa, FcγRIIa, and FcγRI compared to that of an unmodified antibody of the same IgG isotype. In particular embodiments, the HC constant domain or Fc domain of such antibodies is a human HC constant domain or Fc domain.

In particular embodiments, the effector-silent antibodies comprise an Fc domain of an IgG1 isotype that has been modified to lack N-glycosylation of the asparagine (Asn) residue at position 297 (Eu numbering system) of the HC constant domain. The consensus sequence for N-glycosylation is Asn-Xaa-Ser/Thr (wherein Xaa at position 298 is any amino acid except Pro); the N-glycosylation consensus sequence is Asn-Ser-Thr. The modification may be achieved by replacing the codon encoding the Asn at position 297 in the nucleic acid molecule encoding the HC constant domain with a codon encoding another amino acid, for example Ala, Asp, Gln, Gly, or Glu, e.g., N297A, N297Q, N297G, N297E, or N297D. Alternatively, the codon for Ser at position 298 may be replaced with the codon for Pro or the codon for Thr at position 299 may be replaced with any codon except the codon for Ser. In a further alternative each of the amino acids comprising the N-glycosylation consensus sequence is replaced with another amino acid. Such modified IgG molecules have no measurable effector function. In particular embodiments, these mutated HC molecules may further comprise 1, 2, 3, 4, 5, 6, 7, 8, 9, or 10 additional amino acid substitutions, insertions, and/or deletions, wherein said substitutions may be conservative mutations or non-conservative mutations. In further embodiments, such IgGs modified to lack N-glycosylation at position 297 may further include one or more additional mutations disclosed herein for eliminating measurable effector function.

An exemplary IgG1 HC constant domain mutated at position 297, which abolishes the N-glycosylation of the HC constant domain, is set forth in SEQ ID NO: 48. In particular embodiments, these mutated HC molecules may further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the Fc domain of the IgG1 HC constant domain comprising the effector-silent antibodies is modified to include one or more amino acid substitutions selected from E233P, L234A, L235A, L235E, N297A, N297D, D265S, and β331S (wherein the positions are identified according to Eu numbering) and wherein said HC constant domain is effector-silent. In particular embodiments, the modified IgG1 further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the HC constant domain comprises L234A, L235A, and D265S substitutions (wherein the positions are identified according to Eu numbering). In particular embodiments, the HC constant domain comprises an amino acid substitution at position Pro329 and at least one further amino acid substitution selected from E233P, L234A, L235A, L235E, N297A, N297D, D265S, and β331S (wherein the positions are identified according to Eu numbering). These and other substitutions are disclosed in WO9428027; WO2004099249; WO20121300831, U.S. Pat. Nos. 9,708,406; 8,969,526; 9,296,815; Sondermann et al. Nature 406, 267-273 (2000)).

In particular embodiments of the above, the HC constant domain comprises an L234A/L235A/D265A; L234A/L235A/P329G; L235E; D265A; D265A/N297G; or V234A/G237A/P238S/H268A/V309 μL/A330S/P331S substitutions, wherein the positions are identified according to Eu numbering. In particular embodiments, the HC molecules further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the effector-silent antibodies comprise an IgG1 isotype, in which the Fc domain of the HC constant domain has been modified to be effector-silent by substituting the amino acids from position 233 to position 236 of the IgG1 with the corresponding amino acids of the human IgG2 HC and substituting the amino acids at positions 327, 330, and 331 with the corresponding amino acids of the human IgG4 HC, wherein the positions are identified according to Eu numbering (Armour et al., Eur. J. Immunol. 29(8):2613-24 (1999); Shields et al., J. Biol. Chem. 276(9):6591-604(2001)). In particular embodiments, the modified IgG1 further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the effector-silent antibodies comprise a V_{H} fused or linked to a hybrid human immunoglobulin HC constant domain, which includes a hinge region, a CH2 domain and a CH3 domain in an N-terminal to C-terminal direction, wherein the hinge region comprises an at least partial amino acid sequence of a human IgD hinge region or a human IgG1 hinge region; and the CH2 domain is of a human IgG4 CH2 domain, a portion of which, at its N-terminal region, is replaced by 4-37 amino acid residues of an N-terminal region of a human IgG2 CH2 or human IgD CH2 domain. Such hybrid human HC constant domain is disclosed in U.S. Pat. No. 7,867,491, which is incorporated herein by reference in its entirety.

Exemplary IgG1 HC constant domains contemplated herein include HC constant domains comprising an amino acid sequence selected from the group consisting of amino acid sequences set forth in SEQ ID NO: 42, SEQ ID NO: 43, SEQ ID NO: 44, SEQ ID NO: 45, SEQ ID NO: 46, SEQ ID NO: 47, SEQ ID NO: 48, and SEQ ID NO: 49.

In particular embodiments of the integrin binders disclosed herein, the integrin binder is an antibody comprising an IgG1 Fc domain as disclosed herein, which further comprises a C-terminal lysine or lack either a C-terminal lysine or a C-terminal glycine-lysine dipeptide.

In any one of the embodiments disclosed herein, the light chain may comprise a human kappa light chain constant domain comprising SEQ ID NO: 50.

Scfv Fusion Proteins that Bind Integrin

In particular embodiments, the V_{H} and V_{L} disclosed herein are expressed as an ScFv fusion protein in which the V_{L} and V_{H} domains are linked together by a peptide linker. The peptide linker joins the carboxyl terminus of one variable region domain to the amino terminus of the other variable domain without compromising the fidelity of the V_{H}-V_{L} paring and antigen-binding sites. Thus, the ScFv may comprise a fusion protein in which the C-terminus of a V_{L} is linked by a peptide linker to the N-terminus of a V_{H} or a fusion protein in which the C-terminus of a V_{H} is linked by a peptide linker to the N-terminus of a V_{L}. Peptide linkers for linking the variable domains can vary from 10 to 25 amino acids in length and are typically, but not always, composed of hydrophilic amino acids such as glycine (G) and serine (S) having the structure G4S (SEQ ID NO: 53), for example, (G4S)ₙ, wherein n is 1, 2, 3, 4, or 5 (SEQ ID NO: 54). Peptide linkers of shorter lengths (0-4 amino acids) have also been used; however, ScFv bearing shorter linkers may form multimers. Generally, the (G4S)₃ peptide comprising three repeating G4S units (SEQ ID NO: 55) is used as an ScFv peptide linker (See for example, Leath et al., Int. J. Oncol. 24:765-771 (2004); Holliger et al., Proc. Natl. Acad. Sci. USA 90:6444-6448 (1993); Iliades et al., FEBS Lett. 409:437-441 (1997)).

Exemplary ScFv fusion proteins comprise the structure V_{L}-(G4S)ₙ—V_{H} (“(G4S)ₙ” disclosed as SEQ ID NO: 54) or V_{H}-(G4S)ₙ—V_{L} (“(G4S)ₙ” disclosed as SEQ ID NO: 54) wherein the V_{H} domain comprises a CDR 1 comprising the amino acid sequence set forth in SEQ ID NO: 1, a CDR 2 comprising the amino acid sequence set forth in SEQ ID NO: 2, and a CDR 3 comprising the amino acid sequence set forth in SEQ ID NO: 3; and the V_{L} domain comprises a CDR 1 comprising the amino acid sequence set forth in SEQ ID NO: 4, a CDR 2 comprising the amino acid sequence set forth in SEQ ID NO: 5, and a CDR 3 comprising the amino acid sequence set forth in SEQ ID NO: 6, wherein the CDR sequences are defined by the Kabat numbering scheme. In particular embodiment, n is 1, 2, 3, 4, or 5.

Exemplary ScFv fusion proteins comprise the structure V_{L}-(G4S)ₙ—V_{H} (“(G4S)ₙ” disclosed as SEQ ID NO: 54) or V_{H}-(G4S)ₙ—V_{L} (“(G4S)ₙ” disclosed as SEQ ID NO: 54) wherein the V_{H} domain comprises a CDR 1 comprising the amino acid sequence set forth in SEQ ID NO: 7, a CDR 2 comprising the amino acid sequence set forth in SEQ ID NO: 8, and a CDR 3 comprising the amino acid sequence set forth in SEQ ID NO: 9; and the V_{L} domain comprises a CDR 1 comprising the amino acid sequence set forth in SEQ ID NO: 10, a CDR 2 comprising the amino acid sequence set forth in SEQ ID NO: 11, and a CDR 3 comprising the amino acid sequence set forth in SEQ ID NO: 12, wherein the CDR sequences are defined by the Kabat numbering scheme. In particular embodiment, n is 1, 2, 3, 4, or 5.

Exemplary ScFv fusion proteins comprise the structure V_{L}-(G4S)ₙ—V_{H} (“(G4S)ₙ” disclosed as SEQ ID NO: 54) or V_{H}-(G4S)ₙ—V_{L} (“(G4S)ₙ” disclosed as SEQ ID NO: 54) wherein the V_{H} domain comprises a CDR 1 comprising the amino acid sequence set forth in SEQ ID NO: 13, a CDR 2 comprising the amino acid sequence set forth in SEQ ID NO: 14, and a CDR 3 comprising the amino acid sequence set forth in SEQ ID NO: 15; and the V_{L} domain comprises a CDR 1 comprising the amino acid sequence set forth in SEQ ID NO: 16, a CDR 2 comprising the amino acid sequence set forth in SEQ ID NO: 17, and a CDR 3 comprising the amino acid sequence set forth in SEQ ID NO: 18, wherein the CDR sequences are defined by the Kabat numbering scheme. In particular embodiment, n is 1, 2, 3, 4, or 5.

Exemplary ScFv fusion proteins comprise the structure V_{L}-(G4S)ₙ—V_{H} (“(G4S)ₙ” disclosed as SEQ ID NO: 54) or V_{H}-(G4S)ₙ—V_{L} (“(G4S)ₙ” disclosed as SEQ ID NO: 54) wherein the V_{H} domain comprises a CDR 1 comprising the amino acid sequence set forth in SEQ ID NO: 19, a CDR 2 comprising the amino acid sequence set forth in SEQ ID NO: 20, and a CDR 3 comprising the amino acid sequence set forth in SEQ ID NO: 21; and the V_{L} domain comprises a CDR 1 comprising the amino acid sequence set forth in SEQ ID NO: 22, a CDR 2 comprising the amino acid sequence set forth in SEQ ID NO: 23, and a CDR 3 comprising the amino acid sequence set forth in SEQ ID NO: 24, wherein the CDR sequences are defined by the Kabat numbering scheme. In particular embodiment, n is 1, 2, 3, 4, or 5.

Exemplary ScFv fusion proteins comprise the structure V_{L}-(G4S)ₙ—V_{H} (“(G4S)ₙ” disclosed as SEQ ID NO: 54) or V_{H}-(G4S)ₙ—V_{L} (“(G4S)ₙ” disclosed as SEQ ID NO: 54) wherein the V_{H} domain comprises a CDR 1 comprising the amino acid sequence set forth in SEQ ID NO: 25, a CDR 2 comprising the amino acid sequence set forth in SEQ ID NO: 26, and a CDR 3 comprising the amino acid sequence set forth in SEQ ID NO: 27; and the V_{L} domain comprises a CDR 1 comprising the amino acid sequence set forth in SEQ ID NO: 28, a CDR 2 comprising the amino acid sequence set forth in SEQ ID NO: 29, and a CDR 3 comprising the amino acid sequence set forth in SEQ ID NO: 30, wherein the CDR sequences are defined by the Kabat numbering scheme. In particular embodiment, n is 1, 2, 3, 4, or 5.

Exemplary ScFv fusion proteins comprise the structure V_{L}-(G4S)ₙ—V_{H} (“(G4S)ₙ” disclosed as SEQ ID NO: 54) or V_{H}-(G4S)ₙ—V_{L} (“(G4S)ₙ” disclosed as SEQ ID NO: 54) wherein the V_{H} comprises the amino acid sequence set forth in SEQ ID NO: 31 and the V_{L} comprises the amino acid sequence set forth in SEQ ID NO: 32; and, wherein n is 1, 2, 3, 4, or 5.

Exemplary ScFv fusion proteins comprise the structure V_{L}-(G4S)ₙ—V_{H} (“(G4S)ₙ” disclosed as SEQ ID NO: 54) or V_{H}-(G4S)ₙ—V_{L} (“(G4S)ₙ” disclosed as SEQ ID NO: 54) wherein the V_{H} comprises the amino acid sequence set forth in SEQ ID NO: 33 and V_{L} comprises the amino acid sequence set for in SEQ ID NO: 34; and, wherein n is 1, 2, 3, 4, or 5.

Exemplary ScFv fusion proteins comprise the structure V_{L}-(G4S)ₙ—V_{H} (“(G4S)ₙ” disclosed as SEQ ID NO: 54) or V_{H}-(G4S)ₙ—V_{L} (“(G4S)ₙ” disclosed as SEQ ID NO: 54) wherein the V_{H} comprises the amino acid sequence set forth in SEQ ID NO: 35 and the V_{L} comprises the amino acid sequence set for in SEQ ID NO: 36; and, wherein n is 1, 2, 3, 4, or 5.

Exemplary ScFv fusion proteins comprise the structure V_{L}-(G4S)ₙ—V_{H} (“(G4S)ₙ” disclosed as SEQ ID NO: 54) or V_{H}-(G4S)ₙ—V_{L} (“(G4S)ₙ” disclosed as SEQ ID NO: 54) wherein the V_{H} comprises the amino acid sequence set forth in SEQ ID NO: 37 and the V_{L} comprises the amino acid sequence set for in SEQ ID NO: 38; and, wherein n is 1, 2, 3, 4, or 5.

Exemplary ScFv fusion proteins comprise the structure V_{L}-(G4S)ₙ—V_{H} (“(G4S)ₙ” disclosed as SEQ ID NO: 54) or V_{H}-(G4S)ₙ—V_{L} (“(G4S)ₙ” disclosed as SEQ ID NO: 54) wherein V_{H} comprises the amino acid sequence set forth in SEQ ID NO: 39 and the V_{L} comprises the amino acid sequence set for in SEQ ID NO: 40; and, wherein n is 1, 2, 3, 4, or 5.

The ScFvs disclosed herein may be provided in a bispecific format comprising a CD3 binder (ScFv) linked by a peptide linker to an ScFv that binds an HSLN as disclosed herein. When these molecules, called Bispecific T-cell engagers (BiTE®s), bind CD3 on T cells and integrin expressed on the surface of a cell, it brings the T cells to a tumor site.

ScFvs disclosed herein may also be fused to cellular toxins, radioisotopes, cytokines, and enzymes for cancer, autoimmune, and/or inflammatory therapeutic applications. In particular embodiments, the peptide linker may comprise 1 to 10 G4S peptide units (SEQ ID NO: 56).

In further embodiments, the ScFvs disclosed herein may be linked to or inserted in different locations of an intact IgG molecule to confer dual epitope binding. For example, a bispecific antibody may be provided comprising two heterodimeric heavy chain constant domains wherein the N-terminus of one heavy chain constant domain is fused to the C-terminus of an ScFv disclosed herein and the N-terminus of the other heavy chain constant domain is fused to the C-terminus of an ScFv that targets an antigen other than an integrin or a Fab′ that targets an antigen other than an integrin.

### Nucleic Acid Molecules Encoding the Integrin Binders

The present invention further provides nucleic acid molecules that encode the integrin binders of the present invention. In particular embodiments, the integrin binder comprises a V_{H} domain encoded by a nucleic acid molecule and a V_{L} encoded by a nucleic acid molecule. Nucleic acid sequences encoding the integrin disclosed herein may be obtained by back-translating the amino acid sequence of the integrin into a nucleic acid sequence that encodes the integrin. The codons of the nucleic acid molecule so obtained may be further modified to correspond to codons commonly or more efficiently used when translated in a particular cell type. Methods and computer programs for back-translating and/or optimizing a nucleic acid molecule for enhancing expression in a particular host cell are well known in the art, e.g., the IDT Codon Optimization Tool commercially available from Integrated DNA Technologies, Inc. 1710 Commercial Park, Coralville, Iowa 52241, USA.; U.S. Pat. No. 8,326,547; WO2020024917A1.

In particular embodiments, the HC and LC (or V_{H} and V_{L}) are expressed as a fusion protein in which the N-terminus of the HC and the LC (or V_{H} and V_{L}) are fused at the N-terminus to a leader peptide to facilitate the transport of the antibody through the secretory pathway. In particular embodiments, the N-terminus of the ScFv fusion protein is fused at the N-terminus to a leader or signal peptide to facilitate the transport of the ScFv through the secretory pathway. Examples of leader/signal peptides that may be used those comprising the amino acid sequence set forth in SEQ ID NO: 51 or SEQ ID NO: 52. Thus, in particular embodiments, the aforementioned nucleic acid molecules may comprise a polynucleotide encoding a leader peptide linked to the 5′ end of the nucleic acid molecule encoding the integrin.

The nucleic acid molecules disclosed herein may include one or more substitutions that optimize one or more of the codons for enhancing the expression of the nucleic acid molecule in a particular host cell, e.g., yeast or fungal host cell, non-human mammalian host cell, human host cell, insect host cell, or prokaryote host cell.

### Methods for Making Integrin Binders

The present invention includes recombinant methods for making integrin binders comprising introducing into a host cell (i) an expression vector that encodes the V_{H} and V_{L} of an integrin binder or the HC and LC of an integrin binder, or (ii) two expression vectors, one encoding the V_{H} of an integrin binder or the HC of an integrin binder the other encoding the V_{L} of an integrin binder or the LC of an integrin binder. The nucleic acid molecules or polynucleotides encoding the V_{H}, V_{L}, HC, or LC are operably linked to a promoter and other transcription and translation regulatory sequences. The host cell is cultured under conditions and a time period suitable for expression of the nucleic acid molecules followed by isolating the integrin binder from the host cell and/or medium in which the host cell is grown. See e.g., WO2004041862, WO2006122786, WO2008020079, WO2008142164 or WO2009068627. The expression vector may be a plasmid or viral vector. The invention also relates to hosts or host cells that contain such nucleic acid molecule encoding the integrin binders or components thereof, e.g., solely the V_{H} or HC or solely the V_{L} or HC

Eukaryotic and prokaryotic host cells, including mammalian cells as hosts for expression of the integrin binder are well known in the art and include many immortalized cell lines available from the American Type Culture Collection (ATCC). These include, but are not limited to, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Thus, mammalian host cells include human, mouse, rat, dog, monkey, pig, goat, bovine, horse, and hamster cells. Cell lines of particular preference are selected through determining which cell lines have high expression levels. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia pastoris, Saccharomyces cerevisiae, and Trichoderma reesei. The present invention includes any host cell comprising an integrin binder of the present invention or comprising one or more nucleic acid molecules encoding such an integrin binder or comprising an expression vector that comprises one or more nucleic acid molecules encoding such integrin binder.

Further, expression of an integrin binder from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0216846B1, 0256055B1, 0323997B1, and 0338841B1. Thus, in an embodiment of the invention, the mammalian host cells lack a glutamine synthetase gene and are grown in the absence of glutamine in the medium wherein, however, the nucleic acid molecule encoding the immunoglobulin chain comprises a glutamine synthetase gene which complements the lack of the gene in the host cell. Such host cells containing the integrin binder or nucleic acid(s) or expression vector(s) as discussed herein as well as expression methods, as discussed herein, for making the integrin binder using such a host cell are part of the present invention.

The present invention includes methods for purifying an integrin binder comprising introducing a sample (e.g., culture medium, cell lysate or cell lysate fraction, e.g., a soluble fraction of the lysate) comprising the integrin binder to a purification medium (e.g., cation-exchange medium, anion-exchange medium and/or hydrophobic exchange medium) and either collecting purified integrin binder from the flow-through fraction of said sample that does not bind to the medium; or, discarding the flow-through fraction and eluting bound integrin binder from the medium and collecting the eluate. In an embodiment of the invention, the medium is in a column to which the sample is applied. In an embodiment of the invention, the purification method is conducted following recombinant expression of the integrin binder in a host cell, e.g., wherein the host cell is first lysed and, optionally, the lysate is purified of insoluble materials prior to purification on a medium; or wherein the integrin binder is secreted into the culture medium by the host cell and the medium or a fraction thereof is applied to the purification medium.

In general, glycoproteins produced in a particular cell line or transgenic animal will have a glycosylation pattern that is characteristic for glycoproteins produced in the cell line or transgenic animal. Therefore, the particular glycosylation pattern of an integrin binder will depend on the particular cell line or transgenic animal used to produce the integrin binder. integrin binders comprising only non-fucosylated N-glycans are part of the present invention and may be advantageous, because non-fucosylated antibodies have been shown to typically exhibit more potent efficacy than their fucosylated counterparts both in vitro and in vivo (See for example, Shinkawa et al., J. Biol. Chem. 278: 3466-3473 (2003); U.S. Pat. Nos. 6,946,292 and 7,214,775). These integrin binders with non-fucosylated N-glycans are not likely to be immunogenic because their carbohydrate structures are a normal component of the population that exists in human serum IgG.

The present invention includes integrin binders comprising N-linked glycans that are typically added to immunoglobulins produced in Chinese hamster ovary cells (CHO N-linked glycans) or to engineered yeast cells (engineered yeast N-linked glycans), such as, for example, Pichia pastoris. For example, in an embodiment of the invention, the integrin binder comprises one or more of the “engineered yeast N-linked glycans” or “CHO N-linked glycans” (e.g., G0 and/or G0-F and/or G1 and/or G1-F and/or G2-F and/or Man₅). In an embodiment of the invention, the integrin binder comprises the engineered yeast N-linked glycans, i.e., G0 and/or G1 and/or G2, optionally, further including Man5. In an embodiment of the invention, the integrin binders comprise the CHO N-linked glycans, i.e., G0-F, G1-F and G2-F, optionally, further including G0 and/or G1 and/or G2 and/or Man5. In an embodiment of the invention, about 80% to about 95% (e.g., about 80-90%, about 85%, about 90% or about 95%) of all N-linked glycans on the integrin binders are engineered yeast N-linked glycans or CHO N-linked glycans. See Nett et al. Yeast. 28: 237-252 (2011); Hamilton et al. Science. 313: 1441-1443 (2006); Hamilton et al. Curr Opin Biotechnol. 18(5): 387-392 (2007). For example, in an embodiment of the invention, an engineered yeast cell is GFI5.0 or YGLY8316 or strains set forth in U.S. Pat. No. 7,795,002 or Zha et al. Methods Mol Biol. 988: 31-43 (2013). See also international patent application publication no. WO2013066765.

### Administration Pharmaceutical Compositions

The integrin binder disclosed herein may be provided in suitable pharmaceutical compositions comprising the integrin binder and a pharmaceutically acceptable carrier. The carrier may be a diluent, adjuvant, excipient, or vehicle with which the integrin binder is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules or of the invention in such pharmaceutical formulation may vary widely, i.e., from less than about 0.5%, usually to at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21.sup.st Edition, Troy, D. B. ed., Lipincott Williams and Wilkins, Philadelphia, Pa. 2006, Part 5, Pharmaceutical Manufacturing pp 691-1092, see especially pp. 958-989.

The mode of administration of the integrin binder may be any suitable route such as parenteral administration, e.g., intradermal, intramuscular, intraperitoneal, intravenous or subcutaneous, pulmonary, transmucosal (oral, intranasal, intravaginal, rectal) or other means appreciated by the skilled artisan, as well known in the art.

The integrin binder may be administered to an individual (e.g., patient) by any suitable route, for example parentally by intravenous (i.v.) infusion or bolus injection, intramuscularly or subcutaneously, or intraperitoneally. i.v. infusion may be given over for, example, 15, 30, 60, 90, 120, 180, or 240 minutes, or from 1, 2, 3, 4, 5, 6, 7, 8, 9, 10, 11 or 12 hours.

The administration of the integrin binder may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

The integrin binder may be administered by maintenance therapy, such as, e.g., once a week for a period of 6 months or more.

The integrin binder may also be administered prophylactically in order to reduce the risk of developing cancer, delay the onset of the occurrence of an event in cancer progression, and/or reduce the risk of recurrence when a cancer is in remission. This may be especially useful in patients wherein it is difficult to locate a tumor that is known to be present due to other biological factors.

The integrin binder may be lyophilized for storage and reconstituted in a suitable carrier prior to use. This technique has been shown to be effective with conventional protein preparations and well known lyophilization and reconstitution techniques can be employed.

### Combination Therapy Treatments

Combination therapies of the present invention comprising an integrin binder disclosed herein and another therapeutic agent (small molecule or antibody) may be used for the treatment any proliferative disease, in particular, treatment of cancer. In particular embodiments, the combination therapy of the present invention may be used to treat melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

In another embodiment, the combination therapy of the present invention may be used to treat pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, or cancer of hematological tissues.

### Combination Therapy Comprising an Integrin Binder and a Chemotherapy Agent

The combination therapy of the present invention may be administered to an individual having a cancer in combination with chemotherapy. The individual may undergo the chemotherapy at the same time the individual is undergoing the combination therapy of the present invention. The individual may undergo the combination therapy of the present invention after the individual has completed chemotherapy. The individual may be administered the chemotherapy after completion of the combination therapy. The combination therapy of the present invention may also be administered to an individual having recurrent or metastatic cancer with disease progression or relapse cancer and who is undergoing chemotherapy or who has completed chemotherapy.

The chemotherapy may include a chemotherapy agent selected from the group consisting of
(i) alkylating agents, including but not limited to, bifunctional alkylators, cyclophosphamide, mechlorethamine, chlorambucil, and melphalan;
(ii) monofunctional alkylators, including but not limited to, dacarbazine, nitrosoureas, and temozolomide (oral dacarbazine);
(iii) anthracyclines, including but not limited to, daunorubicin, doxorubicin, epirubicin, idarubicin, mitoxantrone, and valrubicin;
(iv) cytoskeletal disruptors (taxanes), including but not limited to, paclitaxel, docetaxel, abraxane, and taxotere;
(v) epothilones, including but not limited to, ixabepilone, and utidelone;
(vi) histone deacetylase inhibitors, including but not limited to, vorinostat, and romidepsin;
(vii) inhibitors of topoisomerase 1, including but not limited to, irinotecan, and topotecan;
(viii) inhibitors of topoisomerase ii, including but not limited to, etoposide, teniposide, and tafluposide;
(ix) kinase inhibitors, including but not limited to, bortezomib, erlotinib, gefitinib, imatinib, vemurafenib, and vismodegib;
(λ) nucleotide analogs and precursor analogs, including but not limited to, azacitidine, azathioprine, fluoropyrimidines (e.g., such as capecitabine, carmofur, doxifluridine, fluorouracil, and tegafur) cytarabine, gemcitabine, hydroxyurea, mercaptopurine, methotrexate, and tioguanine (formerly thioguanine);
(xi) peptide antibiotics, including but not limited to, bleomycin and actinomycin; a platinum-based agent, including but not limited to, carboplatin, cisplatin, and oxaliplatin;
(xii) retinoids, including but not limited to, tretinoin, alitretinoin, and bexarotene; and
xiii) vinca alkaloids and derivatives, including but not limited to, vinblastine, vincristine, vindesine, and vinorelbine.

Selecting a dose of the chemotherapy agent for chemotherapy depends on several factors, including the serum or tissue turnover rate of the entity, the level of symptoms, the immunogenicity of the entity, and the accessibility of the target cells, tissue or organ in the individual being treated. The dose of the additional therapeutic agent should be an amount that provides an acceptable level of side effects. Accordingly, the dose amount and dosing frequency of each additional therapeutic agent will depend in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dose regimen may be made by the clinician, e.g., using parameters or factors known or suspected in the art to affect treatment or predicted to affect treatment, and will depend, for example, the individual's clinical history (e.g., previous therapy), the type and stage of the cancer to be treated and biomarkers of response to one or more of the therapeutic agents in the combination therapy.

Thus, the present invention contemplates embodiments of the combination therapy of the present invention that further includes a chemotherapy step comprising platinum-containing chemotherapy, pemetrexed and platinum chemotherapy or carboplatin and either paclitaxel or nab-paclitaxel. In particular embodiments, the combination therapy with a chemotherapy step may be used for treating at least NSCLC and HNSCC.

The combination therapy further in combination with a chemotherapy step may be used for the treatment any proliferative disease, in particular, treatment of cancer. In particular embodiments, the combination therapy of the present invention may be used to treat melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

In another embodiment, the combination therapy further in combination with a chemotherapy step may be used to treat pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, or cancer of hematological tissues.

In particular embodiments, the combination therapy with a chemotherapy step may be used to treat one or more cancers selected from melanoma (metastatic or unresectable), primary mediastinal large B-cell lymphoma (PMBCL), urothelial carcinoma, MSIHC, gastric cancer, cervical cancer, hepatocellular carcinoma (HCC), Merkel cell carcinoma (MCC), renal cell carcinoma (including advanced), and cutaneous squamous carcinoma.

### Combination Therapy Comprising an Integrin Binder and a Therapeutic Antibody

The integrin binder of the present invention may be administered in combination with one or more therapeutic agents, which may be an antibody, for treatment of cancer or proliferative disease. The individual may undergo treatment with the therapeutic antibody at the same time the individual is undergoing the combination therapy of the present invention. The individual may undergo the combination therapy of the present invention after the individual has completed treatment with the therapeutic antibody. The individual may be administered the treatment with the therapeutic antibody after completion of the combination therapy. The combination therapy of the present invention may also be administered to an individual having recurrent or metastatic cancer with disease progression or relapse cancer and who is undergoing chemotherapy or who has completed chemotherapy. In particular embodiments, the therapeutic agent targets the programmed death 1 receptor or ligand, PD-1 and PD-L1, respectively.

Exemplary anti-PD-1 antibodies that may be used in a combination therapy with the integrin binders disclosed herein include any antibody that binds PD-1 and inhibits PD-1 from binding PD-L1 and/or PD-L2. Exemplary anti-PD-1 antibodies that may be used in a combination therapy with the integrin binders disclosed herein include any antibody that binds PD-L1 or PDL-2 and inhibits PD-1 from binding PD-L1 or PD-L2, respectively. In a further embodiment, the exemplary anti-PD-1 antibody is selected from the group consisting of nivolumab, pembrolizumab, and cemiplimab-rwlc. Exemplary antibodies include the following anti-PD-1 antibodies and compositions comprising an anti-PD1 antibody and a pharmaceutically acceptable salt.

Pembrolizumab, also known as KEYTRUDA, lambrolizumab, MK-3475 or SCH-900475, is a humanized anti-PD-1 antibody described in U.S. Pat. No. 8,354,509 and WO2009/114335 and disclosed, e.g., in Hamid, et al., New England J. Med. 369 (2): 134-144 (2013).

Nivolumab, also known as OPDIVO, MDX-1106-04, ONO-4538, or BMS-936558, is a fully human IgG4 anti-PD-1 antibody described in WO2006/121168 and U.S. Pat. No. 8,008,449.

Cemiplimab-rwlc, also known as cemiplimab, LIBTAYO or REGN2810, is a recombinant human IgG4 monoclonal antibody that is described in WO2015112800 and U.S. Pat. No. 9,987,500.

In particular embodiments, the anti-PD-1 antibody comprises (i) a V_{H} comprising the three HC-CDRs of pembrolizumab fused or linked to an effector-silent HC constant domain and (ii) a V_{L} comprising the three LC-CDRs of pembrolizumab fused or linked to a LC kappa or lambda constant domain.

In particular embodiments, the anti-PD-1 antibody comprises (i) a V_{H} comprising the three HC-CDRs of nivolumab fused or linked to an effector-silent HC constant domain and (ii) a V_{L} comprising the three LC-CDRs of nivolumab fused or linked to a LC kappa or lambda constant domain.

In particular embodiments, the anti-PD-1 antibody comprises (i) a V_{H} comprising the three HC-CDRs of cemiplimab-rwlc fused or linked to an effector-silent HC constant domain and (ii) a V_{L} comprising the three LC-CDRs of nivolumab fused or linked to a LC kappa or lambda constant domain.

In particular embodiments, the anti-PD-1 antibody V_{H} may be fused or linked to an IgG1, IgG2, IgG3, or IgG4 HC constant domain that has been modified to include one or more mutations in the Fc domain that render the resulting anti-PD-1 antibody effecter-silent.

### Injection Device for Administering an Integrin Binder or Composition

The present invention also provides an injection device comprising any one of the integrin binders or compositions disclosed herein. An injection device is a device that introduces a substance into the body of a patient via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., comprising the integrin binder or composition disclosed herein), a needle for piecing skin and/or blood vessels for injection of the fluid; and a plunger for pushing the fluid out of the cylinder and through the needle bore. In an embodiment of the invention, an injection device that comprises the integrin binder or composition is an intravenous (IV) injection device. Such a device includes the integrin binder or composition in a cannula or trocar/needle which may be attached to a tube which may be attached to a bag or reservoir for holding fluid (e.g., saline; or lactated ringer solution comprising NaCl, sodium lactate, KCl, CaCl₂ and optionally including glucose) introduced into the body of the subject through the cannula or trocar/needle.

The integrin binder or composition may, in an embodiment of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an embodiment of the invention, an injection device is an autoinjector; a jet injector or an external infusion pump. A jet injector uses a high-pressure narrow jet of liquid which penetrate the epidermis to introduce the integrin binder or composition to a patient's body. External infusion pumps are medical devices that deliver the integrin binder or composition into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates

### Kits Comprising an Integrin Binder or Composition

Further provided are kits comprising one or more components that include, but are not limited to, an integrin binder or composition as discussed herein in association with one or more additional components including, but not limited to, a further therapeutic agent, as discussed herein. The integrin binder or composition and/or the therapeutic agent can be formulated as a pure composition or in combination with a pharmaceutically acceptable carrier, in a pharmaceutical composition.

In one embodiment, the kit includes an integrin binder or composition in one container (e.g., in a sterile glass or plastic vial) and a further therapeutic agent in another container (e.g., in a sterile glass or plastic vial).

In another embodiment, the kit comprises a combination of the invention, including an integrin binder or composition in combination with one or more therapeutic agents formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above. Thus, the present invention includes a kit comprising an injection device and the integrin binder or composition, e.g., wherein the injection device includes the integrin binder disclosed herein or composition, or wherein the integrin binder or composition is in a separate vessel.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

The examples describe the discovery a new class of anti-integrin monoclonal antibodies that may inhibit integrin-ligand binding, integrin-mediated cell adhesion, and TGF-β1 signaling. The antibodies exhibit distinct human and mouse cross-reactivity and structural modeling predicts a unique mode of inhibition.

### General Methods

Cultured cells and reagents. Normal human lung fibroblasts (Lonza) and Primary Human IPF Lung Parenchymal Fibroblasts (Donor2) (BioIVT, #PCR-70-0214) were incubated at 37° C. and 5% CO₂. To induce fibrotic response in primary cells, a final concentration of 5 ng/mL of recombinant human TGFβ 1 (BioLegend, #580702) was added to the culture media and treated for 24˜48 hours. Anti-TGFβ1 neutralizing antibody (1D11) and the isotype control mouse IgG1 were from BioXcell. SB-525334 and bleomycin were from Sigma. MK-0429 was synthesized by Merck & Co., Inc., Kenilworth, NJ, USA. CHOK1-integrin stable lines were cultured in DMEM/F12, Glutamax™ (Gibco #10565018), 10% FBS (Gibco 310091148), 1×Pen/Strep (Gibco 315140-148), and 6 μg/mL Puromycin (Gibco #A1113803).

Recombinant integrin proteins. The full extracellular domains for all α and β integrin subunits (human and mouse versions, Table 7) were codon optimized for mammalian expression (Genewiz, NJ) and inserted into the HindIII and XhoI sites of pcDNA3.1. αv and α5 contained a C-terminal (GGGS)₃ linker (SEQ ID NO: 57) with an acidic coiled-coil with a cysteine for disulfide-bond formation, a GG-Avitag (Avidity, CO), and a hexa-histidine tag (SEQ ID NO: 58). 01,03, D5, D6, and 38 contained a C-terminal (GGGS)₃ linker (SEQ ID NO: 57) with a basic coiled-coil with a cysteine, and a GG-Avitag.

TABLE 7
Recombinant human and mouse integrin expressing constructs

| Integrin subunit | N-term | C-term |
|---|---|---|
| hαV | 1 | 992 |
| hα5 | 1 | 995 |
| hβ1 | 1 | 728 |
| hβ3 | 1 | 718 |
| hβ5 | 1 | 719 |
| hβ6 | 1 | 709 |
| hβ8 | 1 | 684 |
| mαV | 1 | 988 |
| mα5 | 1 | 999 |
| mβ1 | 1 | 728 |
| mβ3 | 1 | 717 |
| mβ5 | 1 | 719 |
| mβ6 | 1 | 706 |
| mβ8 | 1 | 679 |

For expression, 10 μL of Expi293 cells (ThermoFisher, standard protocol) were co-transfected with 0.5 mg/L each of both an α and a β subunit, either human (h) or mouse (m) and grown for 72 hours at 37° C. in shake flasks (huvPl, hαvβ3, havD5, havD6, hαvβ8, ha5b, mαvβ1, mαvβ3, mαvβ5, mαvβ6, mαvβ8, mα5β1). Media was harvested by centrifugation and soluble supernatant concentrated to 1 μL by TFF (tangential flow filtration, Pellicon 10 K) in 25 mM Tris pH 8.0, 300 mM NaCl, 40 mM Imidazole. Sample was centrifuged again (15 minutes @3500 xg) prior to purification. For expression, the sample was loaded over a HisTrap™ FF (2×5 mL) column pre-equilibrated in 25 mM Tris pH 8.0, 300 mM NaCl, 40 mM Imidazole, washed for 15 column volumes (CV) with the equilibration buffer, and eluted with a gradient from 40 to 500 mM Imidazole (in 25 mM Tris pH 8.0, 300 mM NaCl). Protein not needing biotinylation was further purified using a Superdex™ 200 column in 25 mM TRIS pH 8.0, 150 mM NaCl, 1 mM MgCl₂, 1 mM CaCL₂. Eluted fractions were concentrated to 2 mg/mL, aliquoted into containers and frozen. For biotinylation, sample that needed to be biotinylated after elution from the initial HisTrap™ FF column was concentrated to 3.0 mg/mL and desalted/buffer exchanged (ZebaSpin™ desalting column) into 25 mM Tris pH 8.0, 150 mM NaCl. Sample was combined with Biomix™ B and BirA™ (as per Avidity protocol) and incubated at 4° C. for 14 hours. Biotinylated sample was further purified using a Superdex™ 200 column in 25 mM TRIS pH 8.0, 150 mM NaCl, 1 mM MgCl₂, 1 mM CaCl₂). Eluted fractions were concentrated to 2 mg/mL, aliquoted and frozen. Biotinylation was verified by either streptavidin binding gel shift, or with the Pierce Fluorescence Biotin Quantification Kit (#46610). Antibody Discovery. Optimization, and Production. De Novo Antibody Discovery for antibodies that bind av-integrins was executed on pre-immune yeast display libraries obtained from Adimab LLC. with a diversity of 1010 (Sivasubramanian et al., MAbs 9, 29-42 (2017)). The soluble proteins used in the yeast display selections were biotinylated recombinant integrin ectodomain proteins described in Table 7. All proteins were analytically and biophysically verified by binding against known anti-integrin antibodies (Table 8), size-exclusion chromatography (SEC), Sodium dodecyl sulfate-polyacrylamide gel electrophoresis (SDS-PAGE), and endotoxin analyses.

TABLE 8
Anti-integrin antibodies used for FACS and SALLY SUE simple Western

| Integrin | Antibody | | | | |
|---|---|---|---|---|---|
| target | Clone | Company | Catalog # | Isotype | Reactivity |
| α5 | HMα5-1 | BD | 553350 | hamster IgG1 | mouse |
| α5 | EPR7854 | Abcam | 150361 | rabbit IgG | human, |
| | | | | | mouse |
| α5 | PB1 | DSHB | — | mouse IgG1 | hamster |
| αv | RMV-7 | BD | 552299 | rat IgG1 | mouse |
| αv | P2W7 | R&D Systems | MAB1219 | mouse IgG1 | human |
| β1 | KMI6 | BD | 558741 | rat IgG2a | mouse |
| β1 | P5D2 | R&D Systems | MAB17781 | mouse IgG1 | human |
| β1 | 7E2 | DSHB | — | mouse IgG1 | hamster |
| β3 | HMβ3.1 | BD | 553347 | hamster IgG1 | |
| αvβ3 | 27.1(VNR-1) | Abcam | ab78289 | mouse IgG1 | human |
| β5 | P1F6 | Millipore | MAB1961 | mouse IgG1 | mouse |
| ανβ5 | P5H9 | R&D Systems | MAB2528 | mouse IgG1 | human |
| αvβ6 | 10D5 | BD | 566922 | mouse IgG2a | human, |
| | | | | | mouse |
| β8 | ADWA-11 | Dean Shepard- | — | mouse IgG1 | human, |
| | | UCSF | | | mouse |
| β8 | 416922 | LSBio | LS-C70803 | mouse IgG2b | human |

DSHB—Developmental Studies Hybridoma Bank
BD—BD Biosystems
ADWA-11 - See Stockis et al., Proc. Natl. Acad. Sci. (USA) 114: E10161-E10168 (2017)

Briefly, a yeast IgG library was subjected to multiple rounds of selection by magnetic activated cell sorting (MACS) and florescence activated cell sorting (FACS, BD ARIA III) in phosphate-buffered saline (PBS) buffer containing 1 mM MnCl₂. Selections were performed using 100 nM human or mouse avβ1 followed by rounds of enrichment for populations that were cross-reactive to 100 nM of αvβ3, αvβ5, αvβ6, and αvβ8. Along with the selection progress, the decreased antigen concentrations are also applied to enhance selection pressure to identify higher affinity binders. Isoform-specific selections were achieved by negative sorting of αvβ1 to collect the population that are not binding to α5β1. Top clones were isolated by affinity maturing its parental clone through shuffling the light chain and optimizing heavy chain CDR 1 and CDR 2 sequences. The selection of optimization libraries was repeated using 10 nM αβ1 followed by enrichment for cross-reactivity to αvβ3, αvβ5, αvβ6, and αvβ8, but not α5pl. The isolated clones were then sequenced to identify the unique antibodies and screened for isoform binding profiles by Octet Red. The heavy chain and light chain genes of top clones were cloned into pTT5 mouse Fc-mutated IgG1 backbone vector and produced in Chinese Hamster Ovary (CHO) cells and purified using protein A chromatography. Antibodies were formulated at 2 mg/mL in a buffer composed of 20 mM sodium acetate and 9% sucrose (pH 5.5). Isotype control antibodies was used in subsequent assays.

Integrin cell-based ELISA (CELISA) assay. Cell binding EC₅₀ data for antibodies were obtained for CHOK1 parental cells and CHOK1 cells expressing human or mouse αvβ1, αvβ6, and αvβ1 integrins by cell-based ELISA. Three days prior to the assay cells were seeded at 10,000 cells per well in 100 μL/well of media in Falcon 96-well Flat-Bottom Tissue Culture Plates (REF 353916), resulting in an even cell monolayer in each well on the day of the assay. On the day of the assay, media was removed from the cells and a fivefold titration of primary antibodies was added at concentrations ranging from 0 to 200 nM in TBSF+MnCl₂ buffer (25 mM Tris, 0.15 M NaCl, 0.1% BSA, 0.5 mM MnCl₂, pH 7.5). Cells incubated with primary antibody solution for 1 hour at room temperature. Primary antibody was removed and plates were washed twice with 150 μL/well of 1×DPBS+Tween 20 (TEKnova Cat #β0297, diluted to 1× in distilled water GIBCO #15230-147), before adding 50 μL/well of a 1:5000 dilution of HRP-conjugated goat anti-mouse IgG (Southern Biotech, #1030-05) in TBSF+MnCl₂. After 30 minutes of incubation at room temperature, secondary antibody was removed, and cells were washed twice with 150 μL/well of 1×DPBS+Tween 20. Then 50 μL/well of TMB substrate (1-Step Ultra TMB-ELISA, Thermo Scientific, #34028) was added, incubated with cells at room temperature, and 50 μL/well of TMB stop solution (Seracare, #KPL 50-85-05) was then added after five minutes. Absorbance was read on a TECAN plate reader at 450 nm, with a 620 nm reference wavelength.

Integrin AlphaLISA assay. In the AlphaLISA integrin assays, we used a HEPES based buffer (25 mM HEPES pH 7.4, 137 mM NaCl, 1 mM MgCl₂, 1 mM MnCl₂, 2 mM CaCl₂, 2.7 mM KCl, and 0.05% Tween 20). Optimal assay conditions were determined with a titration of the reagents for signal noise ratio (S/N), linear range, etc. For the respective integrins, we used the final concentrations described in Table 9.

TABLE 9
AlphaLISA assay condition and reagents

| | |
|---|---|
| Integrin EC₈₀ | Acceptor Bead Solution |

| | | | | |
|---|---|---|---|---|
| (batch dependent) | Ligand | antibody | AlphaLISA Acceptor | Donor |

Human AlphalISA Integrin Assays: Final conditions

| | | | | | | | | | | | | | | |
|---|---|---|---|---|---|---|---|---|---|---|---|---|---|---|
| hαvβ1 | 1.4 | nM | human | 1.0 | nM | n/a | | | α - Fibronectin | 10.0 | μg/mL | Streptavidin | 10.0 | μg/mL |
| hα5β1 | 0.4 | nM | Fibronectin | 0.5 | nM | | | | Acceptor beads/ | | | Donor beads/ | | |
| | | | R&D Systems | | | | | | Perkin Elmer | | | Perkin Elmer | | |
| | | | (1918-FN) | | | | | | (CUSM03822000) | | | (6760002) | | |
| | | | | | | | | | See notes | | | | | |
| hαvβ3 | 3.0 | nM | human | 10.0 | nM | n/a | | | α - GST | 15.0 | μg/mL | | 15.0 | μg/mL |
| hαvβ5 | 2.5 | nM | Vitronectin- | 1.0 | nM | | | | Acceptor beads/ | | | | | |
| | | | GST tagged | | | | | | Perkin Elmer | | | | | |
| | | | EMD Millipore | | | | | | (AL110C) | | | | | |
| | | | (08-126) | | | | | | | | | | | |
| hαvβ6 | 0.1 | nM | human LAP | 0.5 | nM | α-Human LAP | 3.0 | nM | α - goat IgG | 15.0 | μg/mL | | 15.0 | μg/mL |
| hαvβ8 | 1.0 | nM | R&D system | 3.0 | nM | (goat pAb) | 3.0 | nM | Acceptor beads/ | | | | | |
| | | | (246-LP) | | | R&D system | | | Perkin Elmer | | | | | |
| | | | | | | (AF-246-NA) | | | (AL107C) | | | | | |

Mouse Alphalisa Integrin Assays: Final Conditions

| | | | | | | | | | | | | | | |
|---|---|---|---|---|---|---|---|---|---|---|---|---|---|---|
| mαvβ1 | 4.3 | nM | mouse | 1.0 | nM | α - Fibronectin | 5.0 | nM | α-rabbit IgG | 15 | μg/mL | Streptavidin | 15 | μg/mL |
| mα5β1 | 1.0 | nM | fibronectin | 2.5 | nM | (Rabbit pAb) | | | Acceptor/ | | | Donor beads/ | | |
| | | | Abcam | | | Abcam | | | Perkin Elmer | | | Perkin Elmer | | |
| | | | (ab92784) | | | (ab2413) | | | (AL104C) | | | (6760002) | | |
| mavβ3 | 3.0 | nM | mouse | 5.0 | nM | α - Vitronectin | 2.0 | nM | | | | | | |
| mαvβ5 | 0.6 | nM | Vitronectin | 2.5 | nM | (Rabbit pAb) | | | | | | | | |
| | | | Abcam | | | Abcam | | | | | | | | |
| | | | (ab92727) | | | (ab140016) | | | | | | | | |
| mαvβ6 | 1.4 | nM | mouse LAP-6His | 2.5 | nM | n/a | | | α-6xHIS/ | 15 | μg/mL | | 15 | μg/mL |
| mavβ8 | 1.0 | nM | Chem partner | 2.5 | nM | | | | Perkin Elmer | | | | | |
| | | | | | | | | | (AL128) | | | | | |

Briefly, the testing antibody (8 μL) was added as a 4×(of Final testing concentration) solution in the HEPES buffer described previously into a 384w AlphaPlate™ (Perkin Elmer #6005350). Then the integrin and ligand were added sequentially as 8×(4 μL each). Plates were sealed and incubated at room temperature for two hours. Then 4×acceptor bead solution (8 L) was added, and the plates were resealed and incubated at room temperature (RT) for one hour. Finally, 4×(8 L) donor beads solution was added in a darkened room and incubated for 45 minutes at room temperature. Plates were read on the Envision (Perkin Elmer) in AlphaScreen™ mode within three hours of donor bead addition.

Cell adhesion assay. For plate preparation, Epic 384-well assay plates (Corning #5040) were washed with OptiMEM™ (Gibco #31985-070) and coated with murine fibronectin (Abcam #ab92784) at a concentration of 0.1 μg/well for CHO-α5KO-mαvβ1 cells; murine vitronectin (Abcam #ab92727) at 0.075 μg/well for CHO-mαvβ3 cells; or murine vitronectin at 0.125 μg/well for CHO-mαvβ5 (all at a volume of 25 μL/well in OptiMEM™), for one hour. Coating solution was then removed by flicking and plates were washed with OptiMEM™ and blocked with 25 μL/well of assay buffer (1% Ovalbumin (Sigma #A5503-10G) in OptiMEM™ containing 1×Pen-Strep (Gibco #15140-148)). A 5-minute baseline reading was taken on a Corning Epica Plate Reader (Model: Epic BT-157900) before removing the assay buffer and adding cells pre-incubated with inhibitors. The Epic™ plate reader detects cellular mass redistribution (adhesion) on the plate surface as a picometer wavelength shift over time. For inhibitor titration preparation: fivefold serial dilutions of small molecule inhibitor MK-0429 or antibody inhibitor Ab-31 were prepared in assay buffer at 4× the final concentration in a 384-well polypropylene plate in a volume of 15 μL per well, starting at a concentration of 800 nM.

For cell preparation: On the day of the assay, frozen cell stocks were thawed and transferred to 10 mL complete media (DMEM/F12+Glutamax™ (Gibco #10565-018) containing 10% FBS (Gibco #10091-148) and 1×Pen-Strep (Gibco 315140-148) for CHO and CHO-mαvβ3 cells, or F12K Nutrient Mixture (Gibco #21127-022) containing 10% FBS (Gibco #10091-148) and 1×Pen-Strep (Gibco #15140-148) for CHO-α5KO-mαvβ3 and CHO-mαvβ3 cells at 37° C. in a 15 mL falcon tube. Cells were pelleted, re-suspended in 10 mL of complete media and allowed to recover for 1.5 hours at 37° C. with gentle shaking. Following cell recovery step, cells were counted using Trypan blue and re-suspended to densities between 26 and 2.676 cells/mL in assay buffer. 45 μL/well of this cell solution was added to the plate containing 15 μL/per well of inhibitor using an Agilent Bravo liquid handler. Final cell concentrations were between 1.56 and 26 cells/mL and final inhibitor concentrations were in a range from 0 to 200 nM. Cells were incubated with the inhibitors for 1 hour at room temperature (RT) with gentle rocking, after which 50 μL per well was transferred to the Epic assay plate, and a time course of cell adhesion was monitored for an additional 3 hours.

Latent TGFβ activation assay. Transfected mink lung epithelial cell (TMLC) cell line containing the TGFβ responsive PAI-1 promoter driving luciferase expression (Mazzieri et al, Methods Mol. Biol. 142, 13-27 (2000)) and the CHO-K1 human and mouse αvβ×integrin cell lines were grown under optimized growth conditions to 90% confluency. TMLC cells were trypsinized and plated at 25,000 cells/well in 50 μL in Costar clear bottom white walled plates. In a separate plate, 25 μL of serially diluted antibodies at 4×concentration were added to wells containing 25 μL of either 25,000 cells of human or mouse αvβ×cells. After a brief mixing, the 50 μL mixture was transferred to the TMLC cell plates for a total volume of 100 μL and incubated at 37° C. for 16 hours. 100 μL of ONE-Glo kit (#PRE6120) was added to each well and read after three to five minutes on the Envision plate reader.

αSMA imaging. Primary cells (passage 3-5) were grown on Cell carrier Ultra, collagen Type 1 coated 96-well plates (Perkin Elmer, #6055700) at a density of 25,000 cells/well. Once the cells adhere, they were starved in serum free media for 24 hours followed by addition of treatments, i.e., 5 ng/mL TGFβ (Biolegend #580702)±inhibitors. Cells were pre-treated with inhibitors for 30 minutes prior to the addition of TGFβ stimulation. 48 hours post treatment, the cells were fixed in 4% methanol free paraformaldehyde (ThermoFisher Scientific #28908) for 20 minutes at room temperature and incubated with anti-alpha smooth muscle Actin antibody (Abcam #ab7817) at 1:4000 overnight at 4° C. Following primary antibody incubation, cells were incubated with Goat anti-mouse secondary antibody (ThermoFisher Scientific #A32723) at 1:500 and Hoechst (ThermoFisher Scientific #62249) at 1:1000 for one hour at RT. The fixed and stained cells were images for immunofluorescence on Perkin Elmer's Opera Phenix high content Imager.

Mouse bleomycin lungfibrosis models. Adult male C57BL/6 mice (Taconic, Rensselaer, NY) were housed in a temperature and humidity-controlled facility with a 12-hour light: 12-hour dark cycle. Animals had ad libitum access to food (Purina Rodent Chow 5053, LabDiet, St. Louis, MO) and water. All procedures utilizing experimental animals were conducted in accordance with the Guide for the Care and Use of Laboratory Animals, and experimental procedures were reviewed and approved by the Institutional Animal Care and Use Committee at MRL, Kenilworth, NJ.

For osmotic minipump implantation, trifluoroacetate (TFA) salt of MK-0429 was formulated in 50% dimethylsulfoxide (DMSO)/50% H₂O at a concentration of 416 mg/mL. MK-0429 or vehicle solution were filled in minipumps (Alzet, #1007D, flow rate 0.5 μL/hour). Minipumps were placed in mice subcutaneously in a pocket on the back between the shoulder blades. A small incision was made and a subcutaneous pocket formed by blunt dissection. A sterile minipump was inserted into the subcutaneous pocket. The incision was closed with staples or non-absorbable suture or absorbable (subcuticular) suture. The minipumps were used for continuous drug delivery for two weeks.

Mice at 12˜13 weeks of age were randomized to five groups: saline, bleomycin instillation with vehicle (osmotic minipump), bleomycin instillation with MK-0429 treatment, bleomycin instillation with vehicle oral treatment, and bleomycin instillation with Nintedanib treatment. All mice were anaesthetized with isoflurane. Bleomycin was dosed by intratracheal (i.t) instillation in a volume of 50 μL, at a dose of 1 unit/kg body weight. After instillation, mice were kept in a heads-up position for 2-5 minutes before putting into cages. MK-0429 (200 mg/kg) was administered by Osmotic pump from day 5 to day 14 and Nintedanib (60 mg/kg) was administered, p.o., q.d., for 14 days. Vehicle was dosed orally at 10 am daily from the day 5 to the end of the studies and dosing volume was 10 mL/kg. For plasma PK analysis, an aliquot of 50 μL plasma was spiked into a 96-well plate, and 200 μL of acetonitrile containing internal standard were added for protein precipitation. The mixture was vortexed, centrifuged at 4000 rpm for 20 minutes. Then 50 μL of supernatant fraction was mixed with 200 μL H₂O and the final solution were injected for LC-MS/MS analysis. The methods for quantitative analysis were developed on UPLC (Waters) chromatographic system equipped with an API4000 QTrap™ mass spectrometer (Applied Biosystems, Concord, Ontario, Canada). Analyst 1.5 software packages (Applied Biosystems) were used to control the LC-MS/MS system and data acquisition and processing.

Histopathology analysis. Upon completion of the study, animals were euthanized and tissues were collected for histological assessment. Lung tissues were perfused with 10% formalin, fixed for 24 hours, and then paraffin embedded. Tissue sections were stained with hematoxylin & eosin (H&E) and Masson's trichrome, subsequently evaluated under light microscope. The severity of histopathologic changes and fibrosis in the lung were graded as described previously by pathologists (Ashcroft et al., J. Clin. Pathol. 41, 467-470 (1988); Hubner et al., Biotechniques 44(507-511), 514-607 (2008)). Following deparaffinization and rehydration, each lung tissue section was processed to identify αSMA deposition by immunohistochemistry. The primary antibodies used were αSMA antibody from Sigma. The Aperio ScanScope™ XT Slide Scanner (Aperio Technologies) system was used to capture whole slide digital images with a 20×objective. Digital images were managed using Aperio Spectrum™. The positive stains were identified and quantified using a macro created from a color deconvolution algorithm (Aperio Technologies). Statistical analysis was performed by using One-way ANOVA followed by Tukey's test.

Integrin co-immunoprecipitation and western blot analyses. Cells or lung tissue were lysed with assay buffer (50 mM Tris, pH7.4, 150 mM NaCl, 1 mM EDTA, 10% glycerol, 2% NP-40) with the addition of protease inhibitor (Roche cOmplete™ mini-pellet, EDTA free, #4693159001) and phosphatase inhibitor cocktails (Sigma #β5726). The total protein concentration was determined by Bradford reagent (Bio-Rad #5000006). One mg of cell lysates were incubated with anti-av antibody (Enzo #ALX-803-304-C100) and Protein G magnetic beads (Pierce #88802) at 4° C. overnight. After washes, the beads were treated with 0.1× sample buffer with fluorescent dye and boiled at 95° C. for 5 minutes. The supernatant was subsequently subjected for SALLY SUE Simple Western analysis from ProteinSimple (Harris, Methods Mol. Biol. 1312, 465-468 (2015)). The antibodies used for detecting integrin isoforms were shown in Table 10, and anti-Focal Adhesion Kinase (FAK) and GAPDH antibodies were from Cell Signaling Transduction.

Molecular modeling. To generate a model for our antibodies, we employed the Antibody Modeler tool within MOE 2019 (Chemical Computing Group, Montreal, Canada) using the determined sequence for Ab-31 and related antibodies to generate a final Fab model. Default settings in MOE 2019 were used during model generation, with the highest scoring model of the ten produced models selected for further study.

To model how our antibodies may bind to integrin, previously published structures of α5β1 integrin (PDB: 3VI4) and αvβ3 (PDB: 6AVQ) were used as models for antibody binding to integrin (Xiong et al., Science 294, 339-345 (2001); Nagae et al., J. Cell Biol. 197, 131-140 (2012)). Each structure was first prepared by deleting additional copies of integrin in the original crystal structures, followed by running the Structure Preparation and Protonate 3D tools within MOE 2019 to add missing side chains, loops, hydrogens, and cap termini using the Amber14EHT force field. Protein-Protein docking was then carried out in MOE 2019 using integrin as the receptor and the modeled antibody Fv region as the ligand while enabling both hydrophobic patch potentials and restraining the ligand site to the CDRs of the modeled Ab-31 structure as annotated by MOE 2019. The resulting poses were then visualized across Ab-31 and related antibodies which were also modeled, as well as across two distinct integrin structures to identify poses that were consistent with the previously described experimental data. The final model was then compared the published integrin-antibody complexes of Abituzumab (PDB: 4002) and LM609 (PDB: 6AVQ) (Mahalingam et al., J. Biol. Chem. 289, 13801-13809 (2014); Borst et al., Structure 25, 1732-1739 (2017)). All images were generated in MOE 2019.

### Example 1

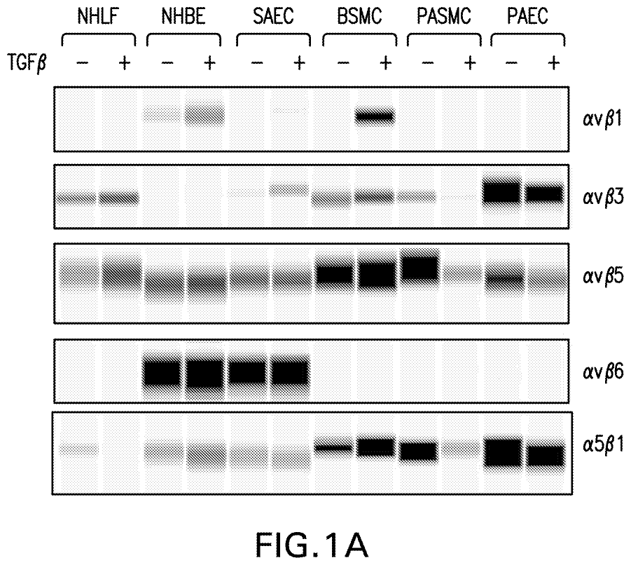

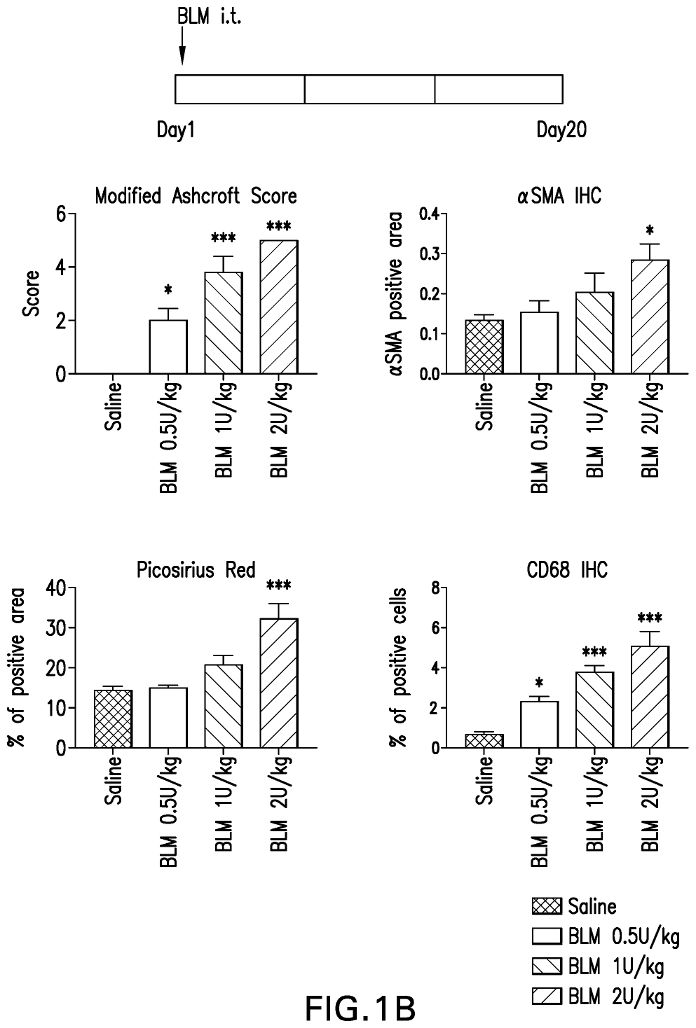

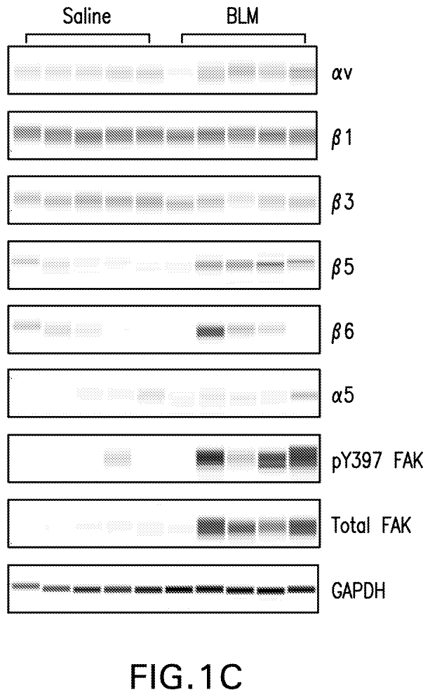

Integrin expression in fibrotic lungs. Recent studies suggested pharmacological targeting of integrins is beneficial in multiple tissue fibrosis, including lung, liver, and kidney fibrosis (Henderson, et al., Nat. Med. 19, 1617-1624 (2013)). To explore the potential role for integrin antagonist in the lung, we first examined the expression of integrins in normal human lung fibroblasts (NHLF), normal human bronchial epithelial cells (NHBE), small airway epithelial cells (SAEC), bronchial smooth muscle cells (BSMC), pulmonary artery smooth muscle cells (PASMC), and pulmonary artery endothelial cells (PAEC). The cells were treated with or without TGFβ, a master regulator of myofibroblast activation and extracellular matrix deposition (Akhurst & Hata, Nat. Rev. Drug. Discov. 11, 790-811 (2012)). The total pool of integrins were immunoprecipitated from the cell lysates and subsequently blotted for the abundance of each 0 subunit. The expression αvβ6 integrin was highly restricted to epithelial cells NHBE and SAEC (FIG. 1A). αvβ6 is a known epithelium-specific integrin and it binds to latent TGFβ in the extracellular matrix, subsequently inducing local activation of the growth factor and promoting fibrosis (Munger et al., Cell 96, 319-328 (1999); Hahm et al., Am. J. Pathol. 170, 110-125 (2007)). Meanwhile, αvβ3 and αvβ5 were more broadly expressed in a variety of lung cell types (FIG. 1A). αvβ1, the less-known member of the integrin family, was barely detected in lung fibroblasts (NHLF) and appeared more abundant in epithelial cells (NHBE) and smooth muscle cells (BSMC) upon TGFβ treatment (FIG. 1A), suggesting that αvβ1 is a highly inducible integrin expressed in multiple cell types. This observation is consistent with our previous report in kidney cell types (Zhou et al., Pharmacol. Res. Perspect. 5(5):e00354 (2017)), pointing to a potential role for αvβ1 integrin beyond fibroblasts.

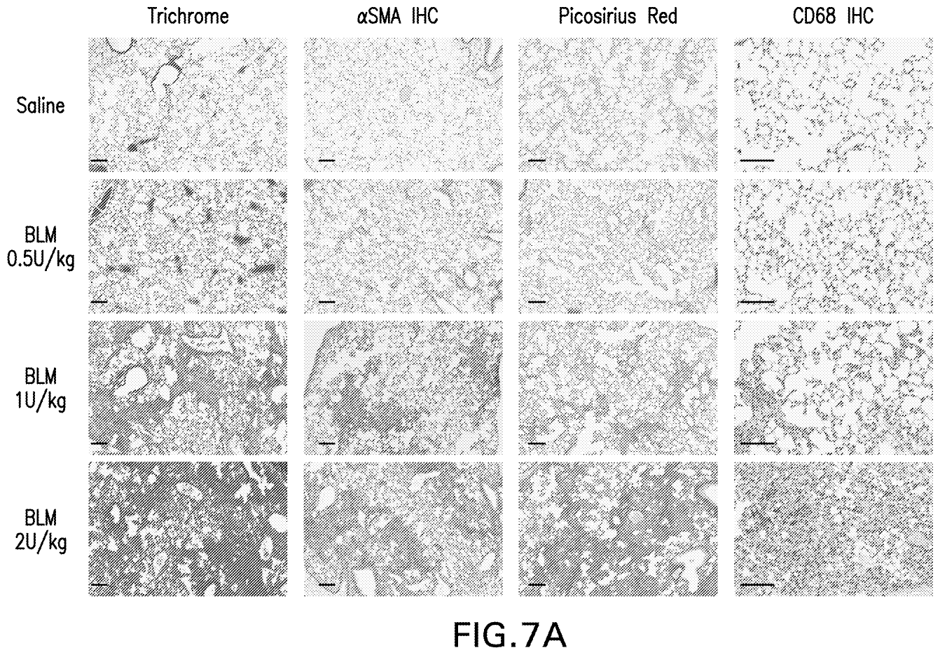

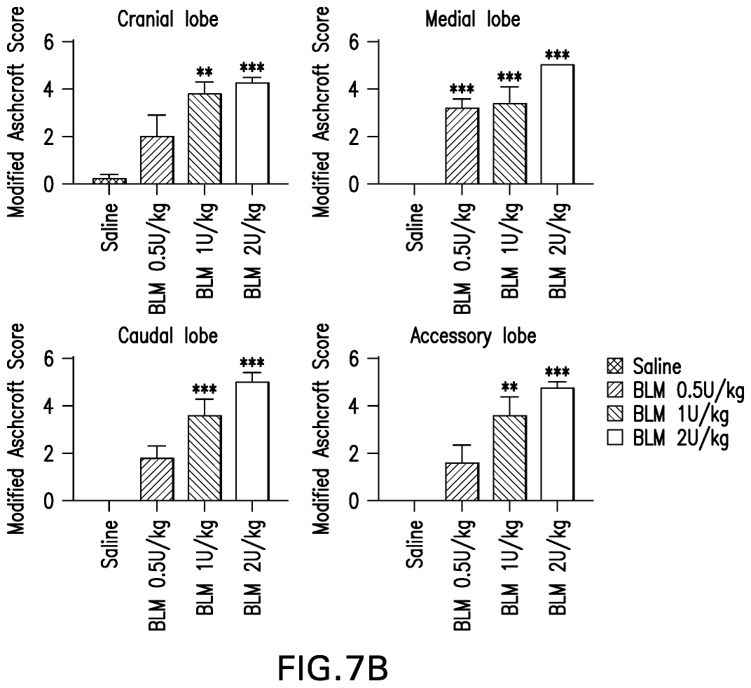

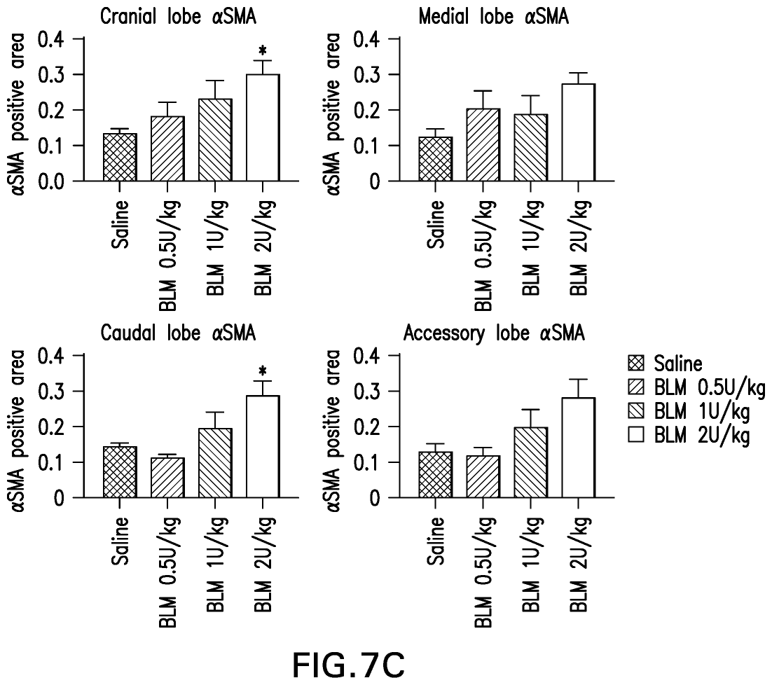

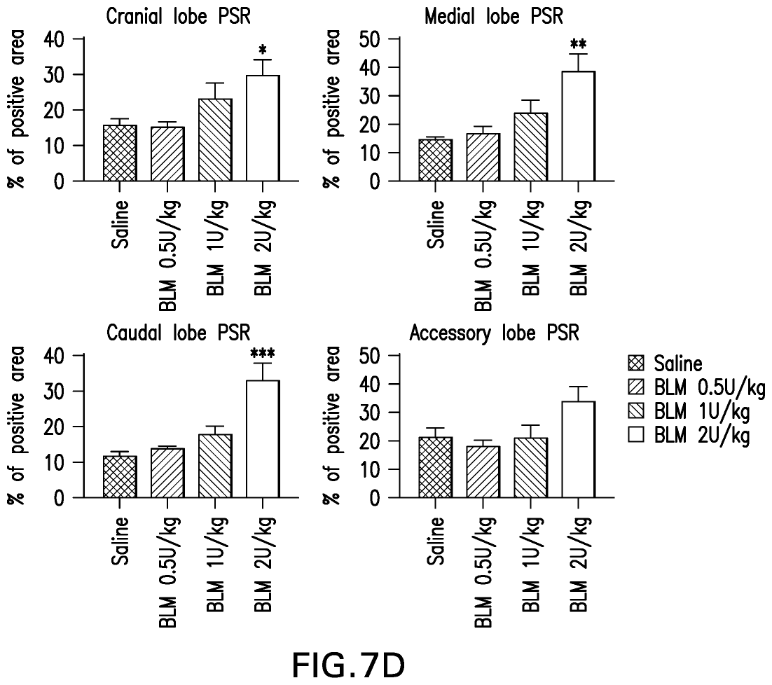

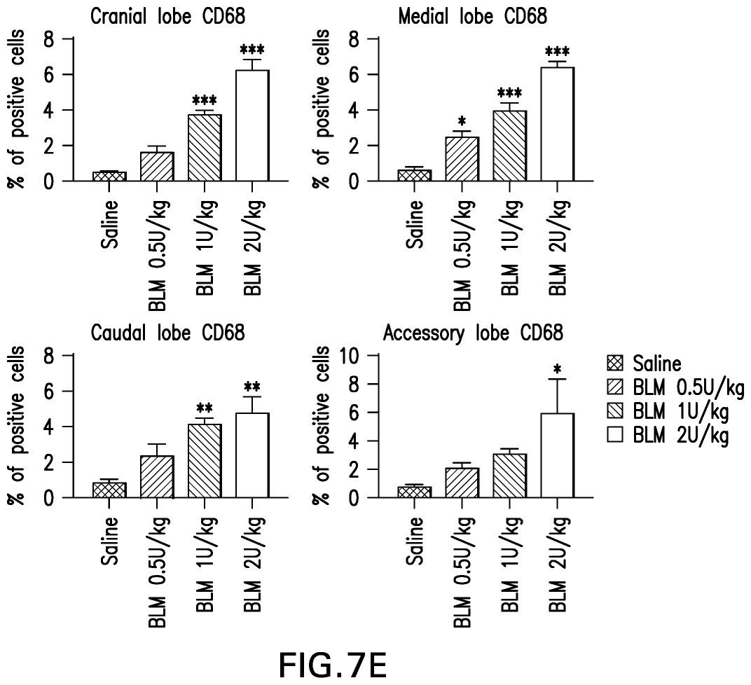

Bleomycin-induced lung fibrosis has been commonly used to evaluate the efficacy of a therapeutic agent in preclinical animal studies. After the initial phase of inflammation and cytokine storm, animals develop fibrosis and progressive lung function decline (Moore et al., Am. J. Respir. Cell. Mol. Biol. 49, 167-179 (2013)). To develop the bleomycin model in mice, we administered various doses of bleomycin via intra-tracheal instillation (FIG. 1B). Twenty days after dosing, lungs were collected for histological evaluation. Bleomycin induced a dose-dependent increase of fibrosis in the lung, as shown by the overall modified Ashcroft score, αSMA-positive cells, and collagen deposition (Picosirius red staining) (FIG. 1B). More cells were reactive to CD68 upon bleomycin administration, indicating significant inflammatory macrophage infiltration. Fibrosis and inflammation were evident across all lung lobes evaluated, suggesting widespread lesions in the lung (FIG. 7A-7B).

To determine the expression of integrins in bleomycin-injured lungs, we next carried out SALLY SUE simple Western analysis of total lung extracts by using a capillary-based electrophoresis system (Harris, Methods Mol. Biol. 1312, 465-468 (2015)). The abundance of αv and β5 integrins was significantly upregulated in bleomycin-treated animals, comparing to those from the saline-treated group (FIG. 1C). 03 integrin expression appeared to be slightly reduced; whereas, β6 and α5 integrin expression was variable among individual animals. Additionally, phospho-Tyr397 and total FAK levels were increased in the bleomycin-treated group, suggesting activation of classical integrin signaling in the lung and providing additional experimental support for integrin targeting in this model.

### Example 2

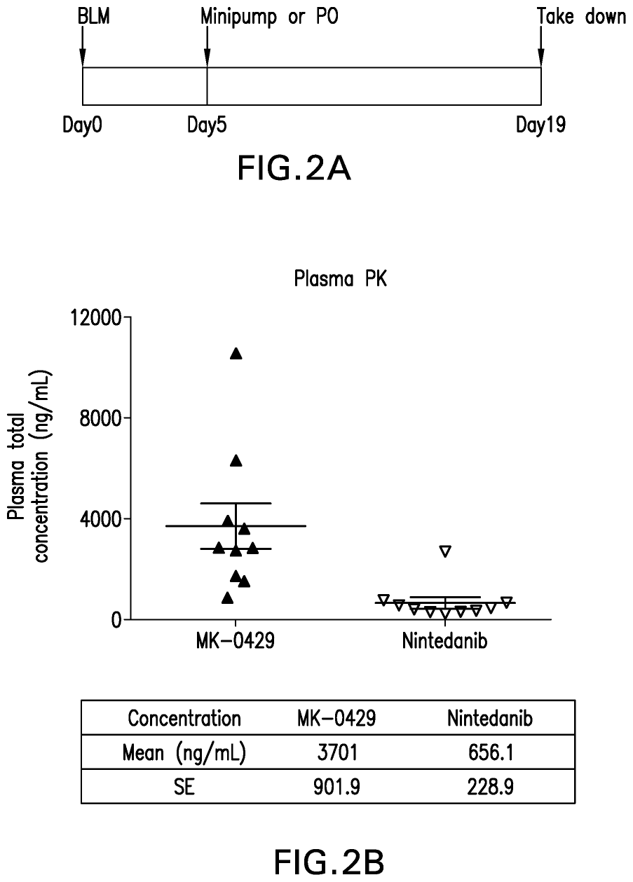

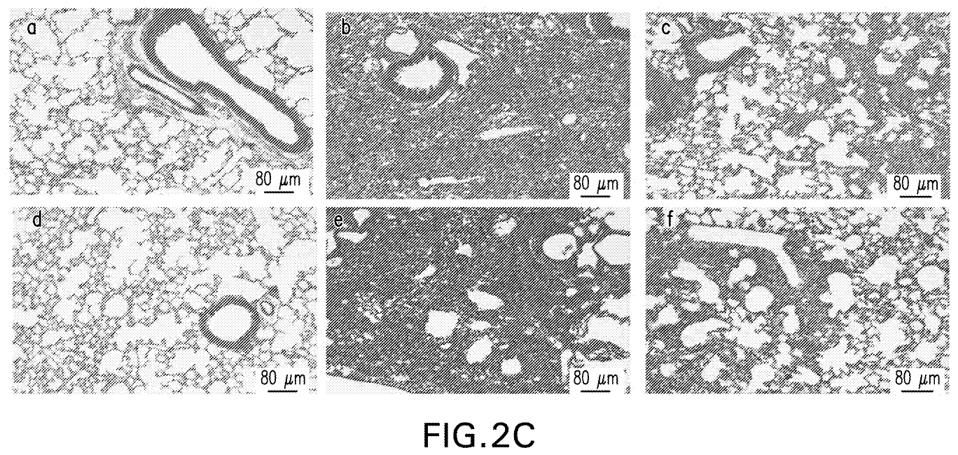

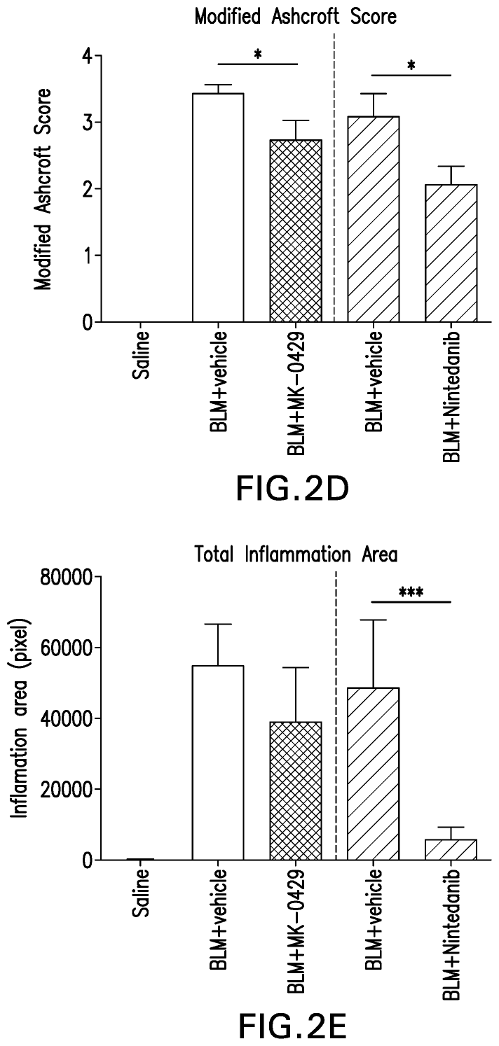

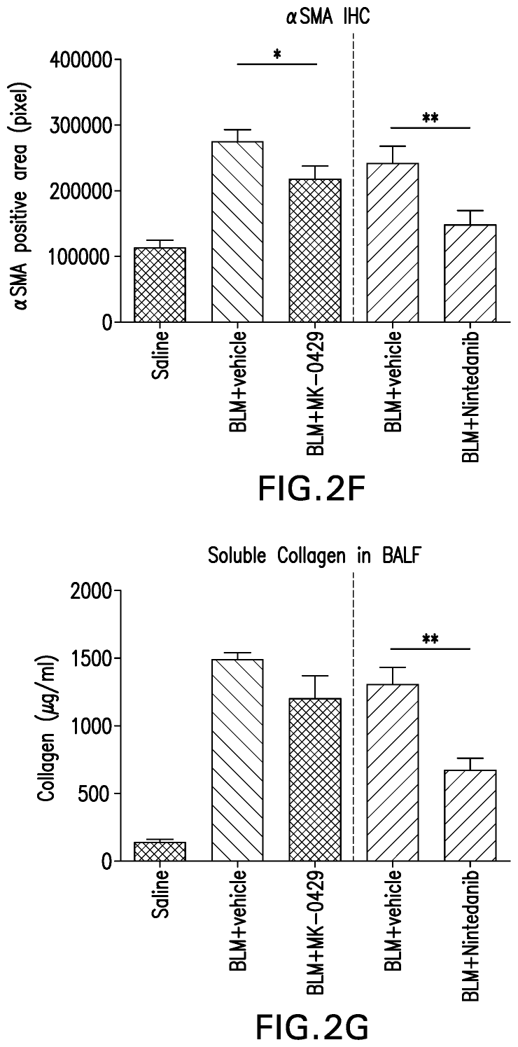

MK-0429 suppresses fibrosis progression in bleomycin model. We previously found that MK-0429 is an equipotent pan-inhibitor of integrins which reduces proteinuria and renal fibrosis in diabetic nephropathy model (Zhou et al., Pharmacol. Res. Perspect. 5(5):e00354 (2017)). We next determined whether MK-0429 could inhibit the progression of lung fibrosis in vivo. Five days after bleomycin administration, we treated mice with MK-0429 (200 mpk via osmotic minipump) or a benchmarking agent (Nintedanib, 60 mpk, po, qd). The animals were analyzed for histology and biomarkers after treatment for 14 days (FIG. 2A). This treatment regimen allowed us to probe the efficacy of the integrin mechanism after bypassing the initial phase of inflammation and cytokine storm.

Plasma concentration levels of MK-0429 and Nintedanib were determined two hours after the last dose at the end of the study. The mean plasma concentration was 3701÷902 ng/mL for MK-0429, and 656+229 ng/mL Nintedanib (FIG. 2B), consistent with the previous report (Zhou et al., Pharmacol. Res. Perspect. 5(5):e00354 (2017)). Notably, this concentration of Nintedanib exceeds the level of no-observed-adverse-effect-level (NOAEL) dose identified in mice from New Drug Application pharmacology review of Nintedanib (U.S. Food and Drug Administration, Center for Drug Evaluation and Research, Clinical Pharmacology and Biopharmaceutics Review, Application No. 20583200rig1s000).

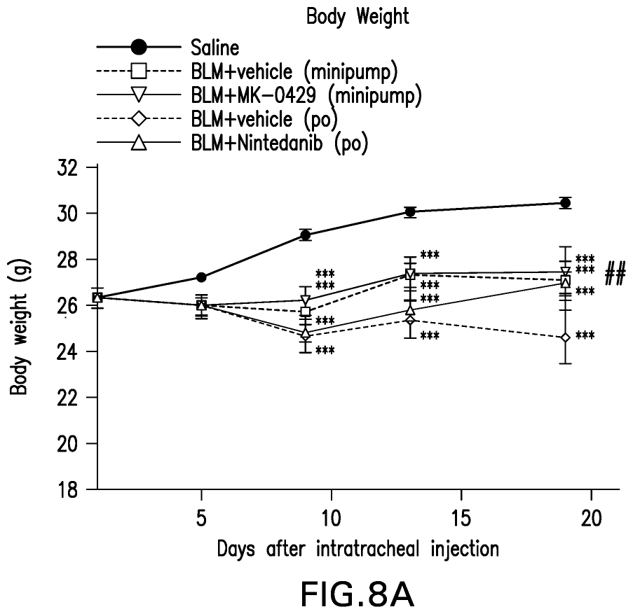

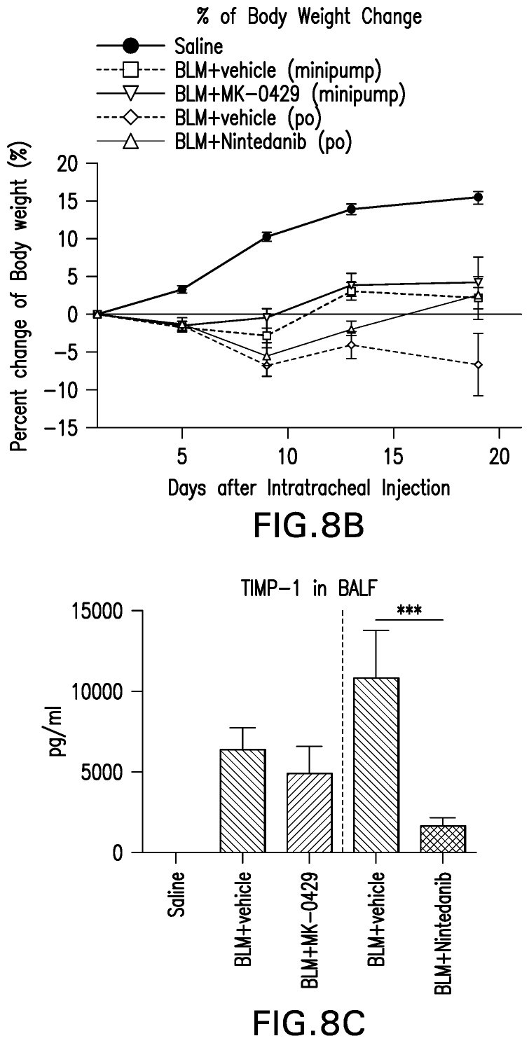

Bleomycin instillation significantly decreased the bodyweight when compared to mice treated with Saline (FIG. 8A-8B). After MK-0429 and Nintedanib administration, there was no significant body weight difference between the bleomycin group and different treatment groups. There was no significant difference for percentage change of body weight among various groups. Mouse lungs were collected at Day 19 for histological evaluation. As shown by the Masson Trichrome staining (FIG. 2C), control lung tissues had normal lung structure with some collagen around the bronchioles (FIG. 2C, panels a and d). Administration of bleomycin caused severe multifocal and diffuse fibrosis, thickening of alveolar septa, intra-alveolar fibrosis, and increased perivascular and peribronchiolar infiltration of inflammatory cells (FIG. 2C, panel b). Nintedanib significantly improved modified Ashcroft score and decreased inflammation after 14 days treatment (FIG. 2C, panel c; FIG. 2D, FIG. 2E). Meanwhile, MK-0429 treatment at 200 mpk significantly decreased the modified Ashcroft score and led to a nonstatistical significant decrease of inflammation in the lung (FIG. 2D, FIG. 2E). Myofibroblast proliferation in lung tissue was detected by αSMA IHC staining. As shown in FIG. 2F, bleomycin significantly increased the immunoreactivity for αSMA, both Nintedanib and MK-0429 significantly decreased bleomycin-induced αSMA expression.

Bronchioalveolar lavage fluid (BALF) was also collected for biomarker analyses Bleomycin increased soluble collagen content and TIMP-1 levels in BALF (FIG. 2G, FIG. 8C). Nintedanib significantly decreased BALF soluble collagen and TIMP-1 content after 14 days treatment. The decreases in BALF soluble collagen and TIMP-1 content upon MK-0429 treatment did not reach statistical significance. Together, our results demonstrate that MK-0429 is effective at reducing fibrosis progression in a bleomycin lung injury model.

### Example 3

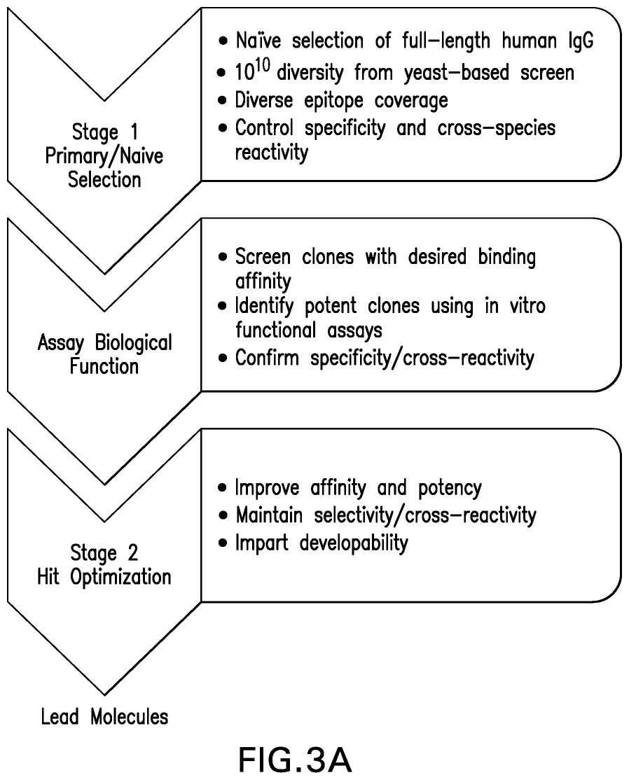

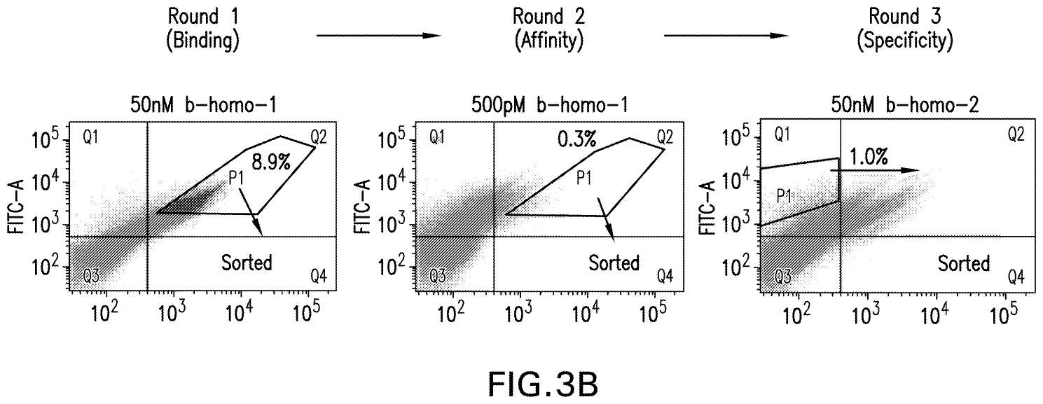

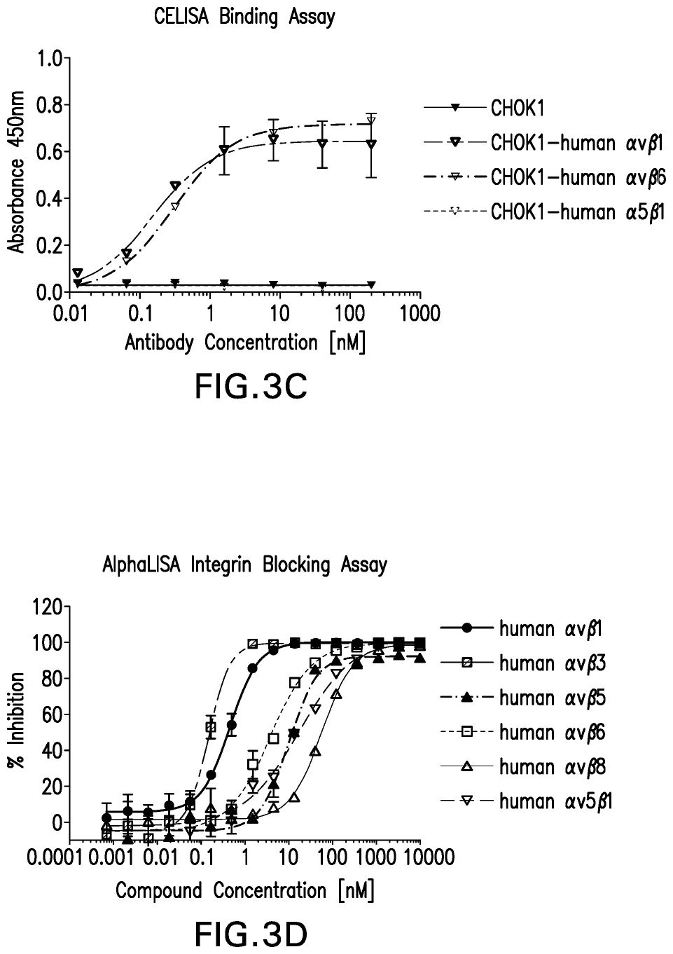

Discovery of novel integrin monoclonal antibodies with human and mouse cross-reactivity. To obtain new integrin inhibitors for fibrosis, we screened a full-length human naïve IgG yeast surface display library to find fully human antibodies with better potency than MK-0429. The library identification and counter selection of αv-integrin antibodies (FIG. 3A). Extracellular domains of recombinant human and mouse αvβ1, αvβ3, αvβ5, αvβ6, αvβ8, and 501 integrin proteins (Table 7) were purified, biotinylated, and used as baits to screen for specific binders through several rounds of enrichment by magnetic bead isolation or fluorescence activated cell sorting (FACS). In round 1 and 2, IgG-presenting yeast library clones were enriched for target binding and affinity using bait αvβ×proteins at a concentration of 50 nM and 500 pM, respectively (FIG. 3B). A yeast cell population with strong antigen binding was sorted out for the next round of selection. In round 3, the best-binders were depleted for αvβ1 binding and PSR (poly-specificity reagent) non-specific binding. From the initial screen, we identified 188 unique binders for IgG expression, purification, and functional characterization.

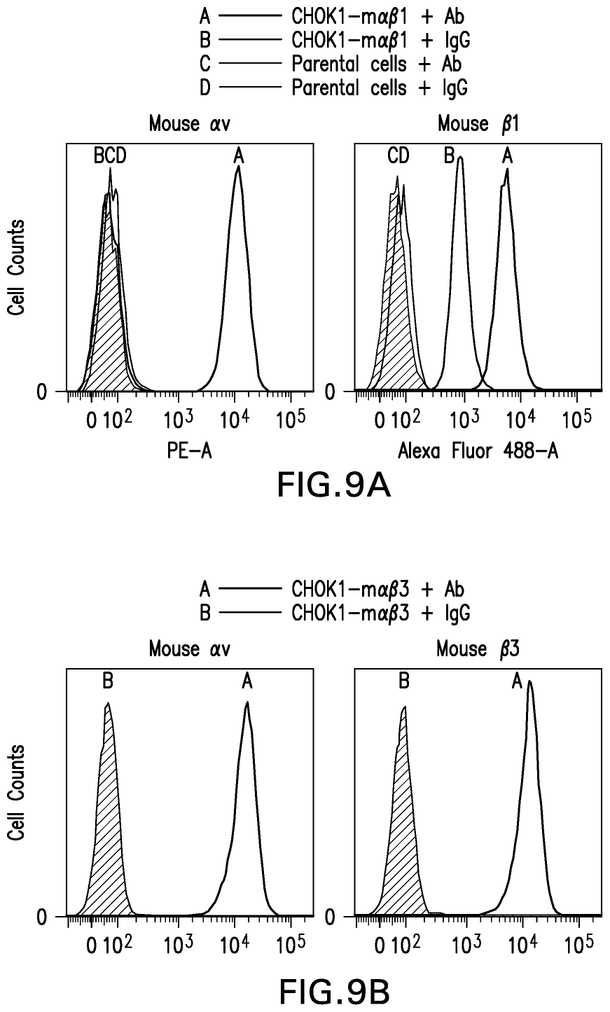

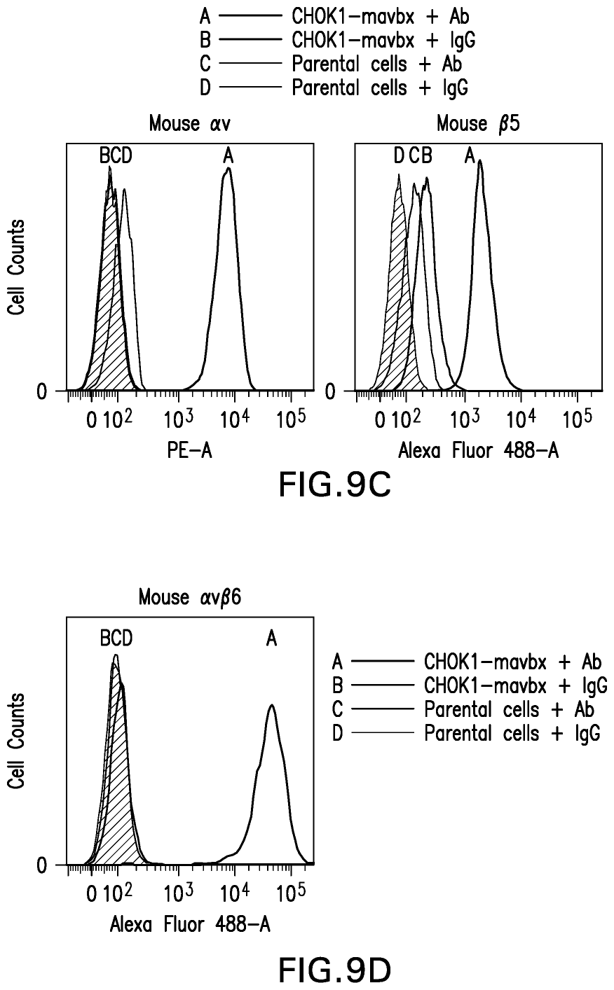

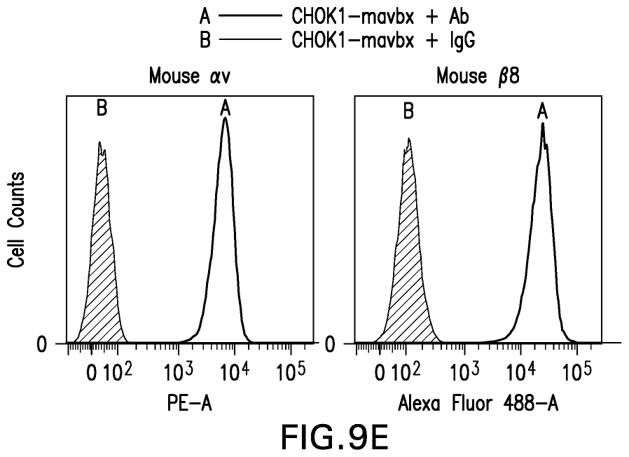

To examine the binding affinity of each clone, we next generated a set of CHOK1 cell lines that stably expressed human or mouse αvβ1. αvβ3, αvβ5, αvβ6, αvβ8, and α5β1 integrins. The relative abundance of each integrin was determined by FACS analyses after staining the cells with corresponding integrin antibodies (FIG. 9, Table 8). The binding affinity and specificity of each yeast clone to CHOK1 parental cells or CHOK1-αvβ1, αvβ6, or α5β1-expressing cells were determined upon antibody titration in a cell-based ELISA (CELISA) assay. As shown in FIG. 3C, control mAb-24 preferentially bound to human αvβ1 and αvβ6 integrin but not α5β1 integrin in this high-throughput cell-based binding assay. 34 antibody clones with ten-fold higher binding affinity towards αvβ1 and αvβ6 integrins were selected for further functional characterization.

We previously determined the in vitro potency and selectivity of MK-0429 by using solid-phase ELISA and thermal shift assays (Zhou et al., Pharmacol. Res. Perspect. 5(5):e00354 (2017)). To develop a more sensitive and high-throughput functional assay to screen integrin antibodies, we utilized the AlphaLISA platform to determine the blocking of integrins-ligand binding by antibodies. It has been shown that fibronectin, vitronectin, and TGFβ latency-associated peptide (LAP) function as endogenous ligands for av$l, αvβ3/5, and αvβ6/8 (Reed, N. I. et al., Sci. Transl. Med. 7, 288 (2015); Ozawa et al., J. Biol. Chem. 291, 11551-11565 (2016); Weinreb et al., J. Biol. Chem. 279, 17875-17887 (2004) ; Vogel et al., J. Cell Biol. 121, 461-468 (1993)). Additionally, α531 is a well-known fibronectin receptor Wu, et al., J. Biol. Chem. 268, 21883-21888 (1993)). Thus, we used different ligands for each integrin (Table 9) and optimized the assay conditions to obtain curve-fitted IC₅₀ values for MK-0429. As shown by the results in FIG. 3D, MK-0429 demonstrated potent inhibition against human αvβ1 (IC₅₀=0.46 nM), αvβ3 (IC₅₀=0.15 nM), αvβ5 (IC₅₀=9.9 nM), αvβ6 (IC₅₀=3.8 nM), αvβ1 (IC₅₀=58.3 nM), and αvβ1 (IC₅₀=17.3 nM), consistent with our previous solid-phase ELISA assay results (Zhou et al., Pharmacol. Res. Perspect. 5(5):e00354 (2017)). Subsequently, we selected strong binders to examine their abilities to block integrin function in both human and mouse AlphaLISA assays.

Our initial screen led to the identification of five unique antibody clones from different germlines that potently inhibited multiple av-integrins. The top clones were selected for light chain shuffling and affinity maturation on heavy chain CDR 1 and CDR 2 sequences. Daughter clones with improved binding affinity and potency were expressed in mammalian cells and purified for further characterization to identify lead molecules (FIG. 3A).

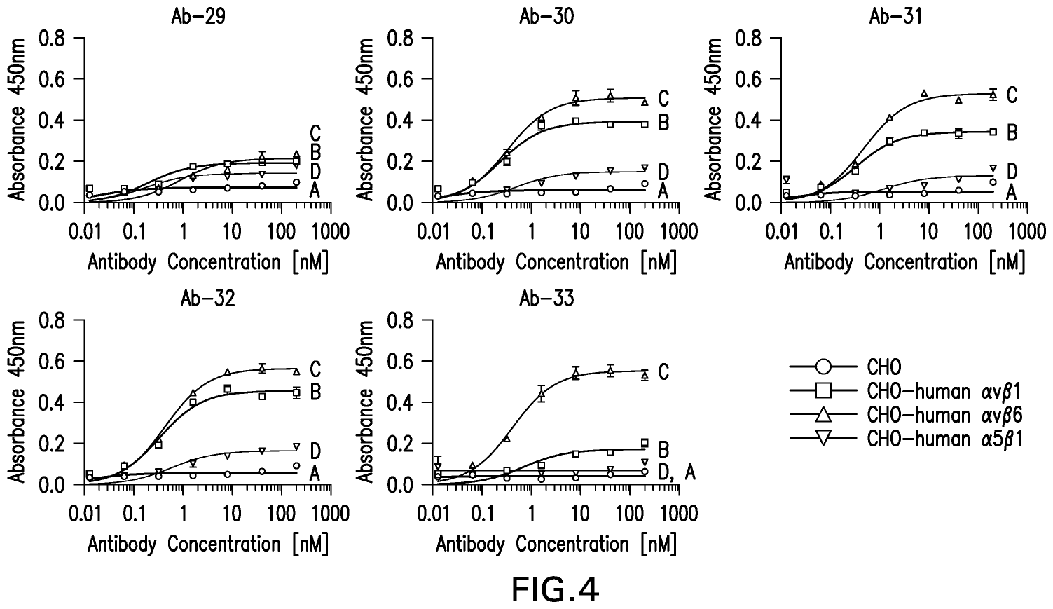

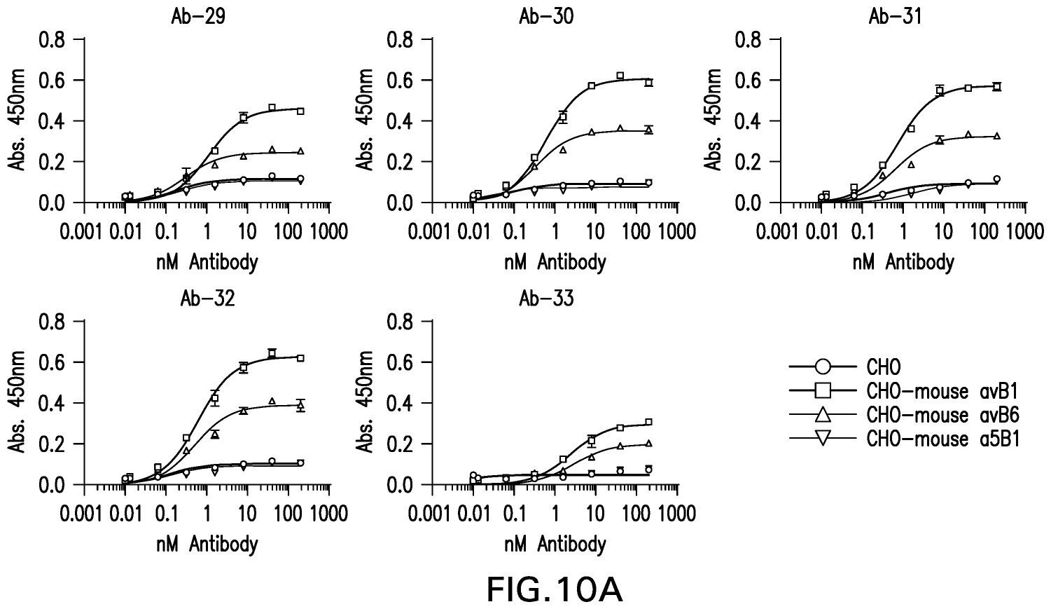

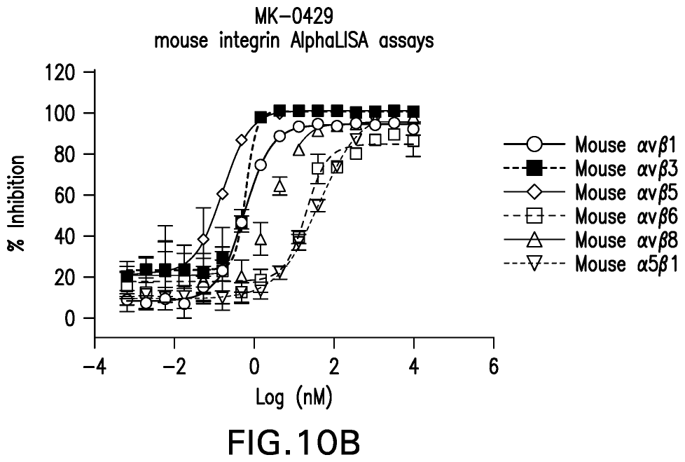

After an initial screening and two rounds of affinity maturation, we identified five unique antibodies, Ab-29, Ab-30, Ab-31, Ab-32, and Ab-33, which strongly bound to human αvβ1 and αvβ6 integrins, and in a lesser extent towards αvβ1 integrin (FIG. 4A). The EC₅₀ of Ab-29, Ab-30, Ab-31, Ab-32, and Ab-33 in cell-based ELISA (CELISA) assays are presented in Table 10, as well as their IC₅₀ in human and mouse AlphaLISA integrin blocking assays, Table 11 and Table 12, respectively. The antibody EC₅₀s of cell-based binding towards each integrin shown in Table 10 suggest that they are pan-av inhibitors. Notably, those five molecules were also reactive to mouse αvβ3l, αvβ36, and α5031 integrins (Table 11 and Table 12, FIG. 10A).

TABLE 10
EC₅₀ of selected antibodies in cell-based ELISA assays

| EC₅₀ or | Cell-based Binding | | | | | |
|---|---|---|---|---|---|---|
| IC₅₀ (nM | hαvβ1 | hαvβ6 | hα5β1 | mαvβ1 | mαvβ6 | mα5β1 |
| Ab-29 | 0.198 | 0.861 | 0.166 | 1.087 | 0.285 | 0.213 |
| Ab-30 | 0.230 | 0.323 | 0.495 | 0.593 | 0.335 | 0.030 |
| Ab-31 | 0.320 | 0.483 | 1.22 | 0.743 | 0.640 | 1.761 |
| Ab-32 | 0.335 | 0.438 | 0.597 | 0.609 | 0.508 | 0.131 |
| Ab-33 | 0.702 | 0.417 | N/A | 2.840 | 2.840 | N/A |
| MK-0429 | — | — | — | — | — | — |
| mAb-24 | — | — | — | — | — | — |

TABLE 11
IC₅₀ of selected antibodies in human
AlphaLISA integrin blocking assays

| EC₅₀ or | Human integrin AlphaLISA assays | | | | | |
|---|---|---|---|---|---|---|
| IC₅₀ (nM | hαvβ1 | hαvβ3 | hαvβ5 | hαvβ6 | hαvβ8 | hα5β1 |
| Ab-29 | 2.57 | 1.161 | 14.78 | 96.02 | 200.30 | 9.46 |
| Ab-30 | 3.15 | 1.24 | 53.51 | 25.41 | 19.74 | 43.10 |
| Ab-31 | 2.26 | 0.94 | 31.73 | 30.89 | 47.79 | 47.34 |
| Ab-32 | 2.41 | 1.03 | 25.99 | 16.40 | 48.00 | 34.07 |
| Ab-33 | 1.27 | 1.28 | 22.27 | 7.49 | 12.17 | 31.12 |
| MK-0429 | 1.0 | 1.6 | 2.9 | 0.5 | 3.1 | 26.5 |
| mAb-24 | 0.93 | 5.62 | 3.50 | 3.64 | 2.91 | >250 |

TABLE 12
IC₅₀ of selected antibodies in human
AlphaLISA integrin blocking assays

| EC₅₀ or | Mouse integrin AlphaLISA assays | | | | | |
|---|---|---|---|---|---|---|
| IC₅₀ (nM | mαvβ1 | mαvβ3 | mαvβ5 | mαvβ6 | mαvβ8 | mα5β1 |
| Ab-29 | 3.4 | 3.4 | 17.7 | 26.3 | 101.9 | >250 |
| Ab-30 | 6.8 | 4.2 | >250 | 23.2 | 25.1 | >250 |
| Ab-31 | 6.6 | 3.2 | >250 | 17.7 | 66.3 | >250 |
| Ab-32 | 6.7 | 4.3 | >250 | 17.1 | 71.9 | >250 |
| Ab-33 | 4.5 | 9.2 | 96.3 | 6.6 | 24.7 | >250 |
| MK-0429 | 1.3 | 0.9 | 48.3 | 0.4 | 5.3 | 50.3 |
| mAb-24 | >250 | >250 | >250 | >250 | >250 | >250 |

To date, there are no αv-integrin antibodies with human and mouse cross-reactivity reported. We next carried out AlphaLISA integrin blocking assays to examine the effect of each antibody on integrin-ligand binding. As shown in Table 10, Ab-29, Ab-30, Ab-31, Ab-32, and Ab-33 substantially inhibited human αvβ1, αvβ3, αvβ5, αvβ6, αvβ8, and α5β1 integrins, with IC₅₀s comparable to those of MK-0429 and control antibody abituzumab (mAb-24). MK-0429 was potent at blocking mouse integrin-ligand binding in all cases except mouse αvβ6 and α5β1, where it showed weaker activities (Table 11 and Table 12, FIG. 10B). Ab-29, Ab-30, Ab-31, Ab-32, and Ab-33 also strongly inhibited mouse αvβ1, αvβ3, αvβ6, and αvβ8 integrins. Their activities towards mouse αvβ1 integrin were negligible. The binding affinity (Kd) of each antibody to selected integrin proteins was within the range of 2-48 nM as assessed by the ForteBio Octet Red™ system (Table 13). Epitope binning revealed unique binding sites for our antibodies compared to other known αv-integrin antibodies. Overall, our molecules represent a new class of αv-integrin blocking antibodies that could be used as mouse surrogates for rodent preclinical studies.

TABLE 13
Octet Red binding parameter of Ab-29, Ab-30, Ab- 31, Ab-
32, and Ab-33 to selected human and mouse integrins

| Clone | hαvβ1 | hαvβ3 | mαvβ1 | mαvβ6 |
|---|---|---|---|---|
| Name | KD (M) | KD (M) | KD (M) | KD (M) |
| Ab-29 | 2.70E−09 | 6.58E−09 | 4.21E−09 | 4.11E−09 |
| Ab-30 | 6.750E−09 | 5.29E−09 | 3.5E−08 | 3.03E−09 |
| Ab-31 | 1.40E−08 | 6.47E−09 | 4.84E−08 | 2.9E−09 |
| Ab-32 | 7.40E−09 | 6.14E−09 | 2.59E−08 | 2.59E−09 |
| Ab-33 | 1.27E−08 | 5.07E−09 | 3.77E−08 | 2.45E−09 |

### Example 4

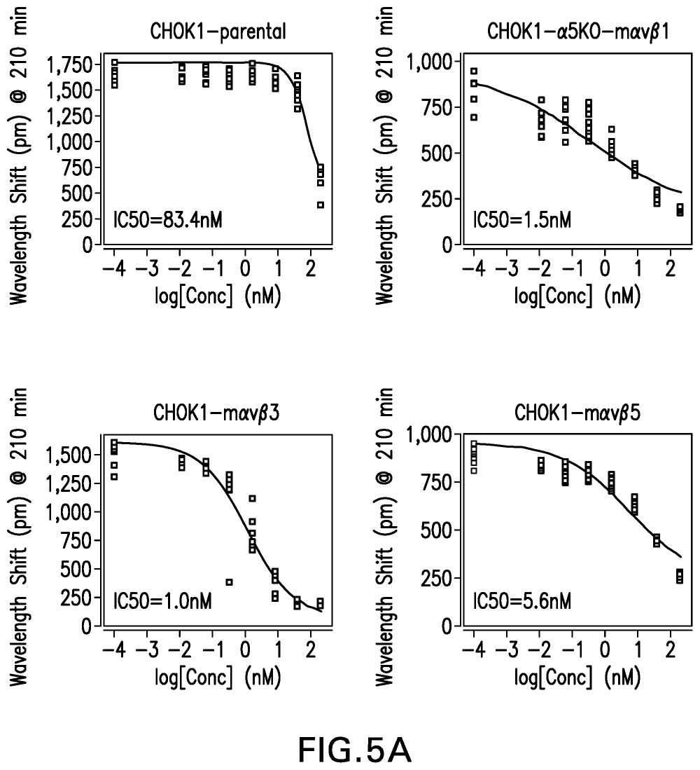

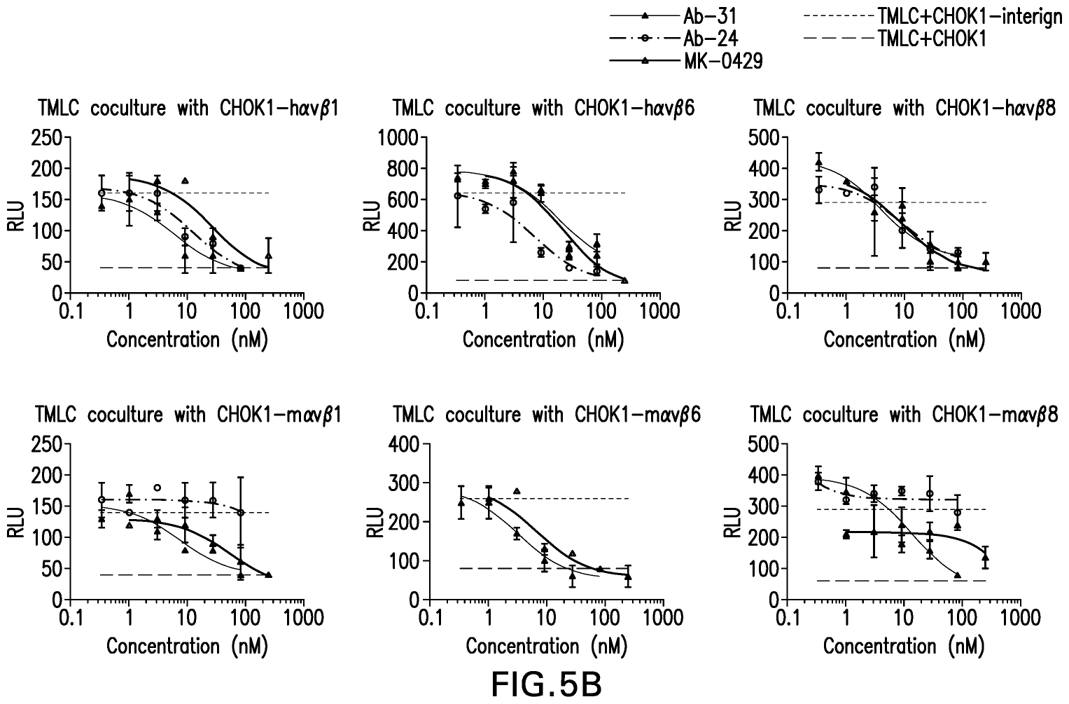

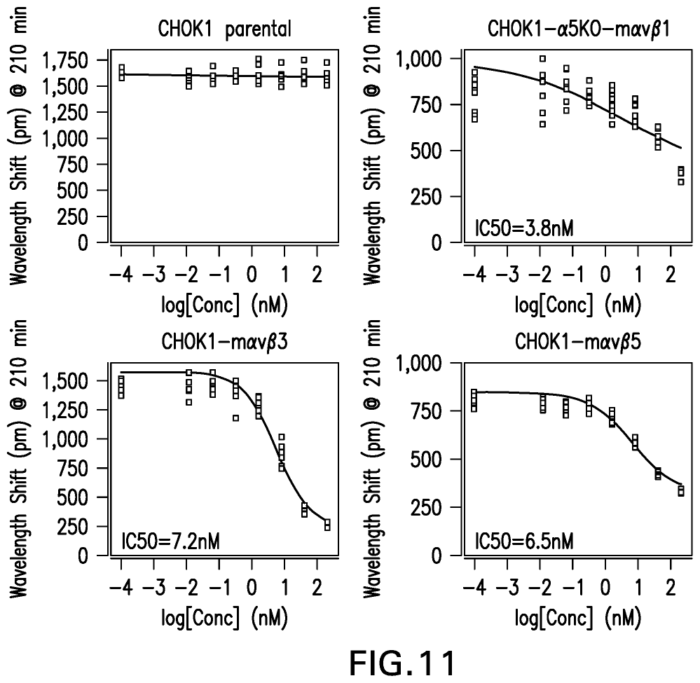

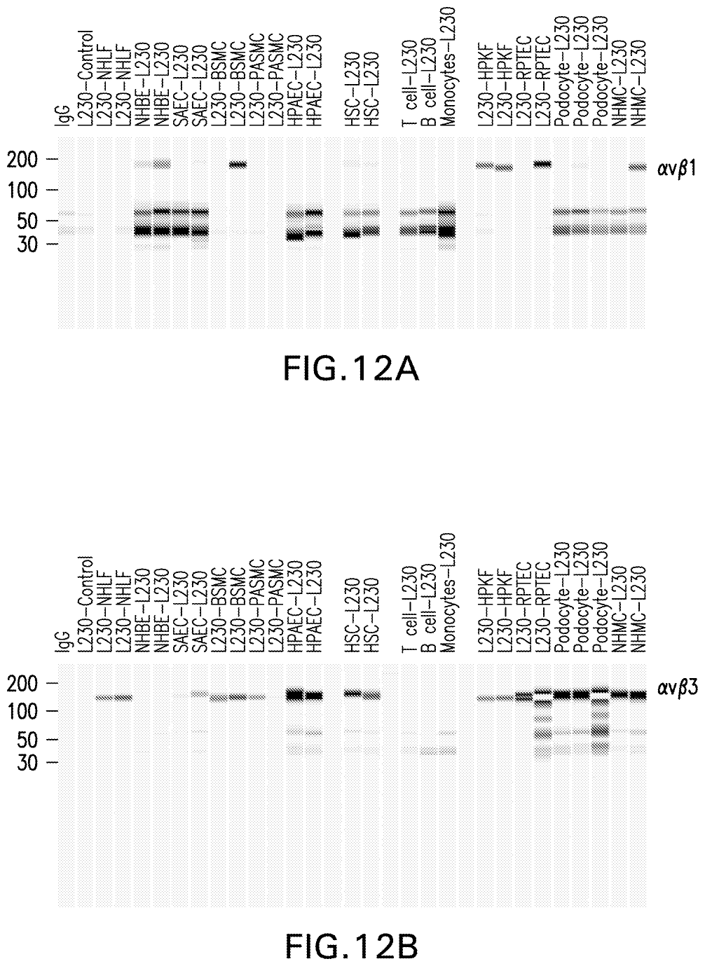

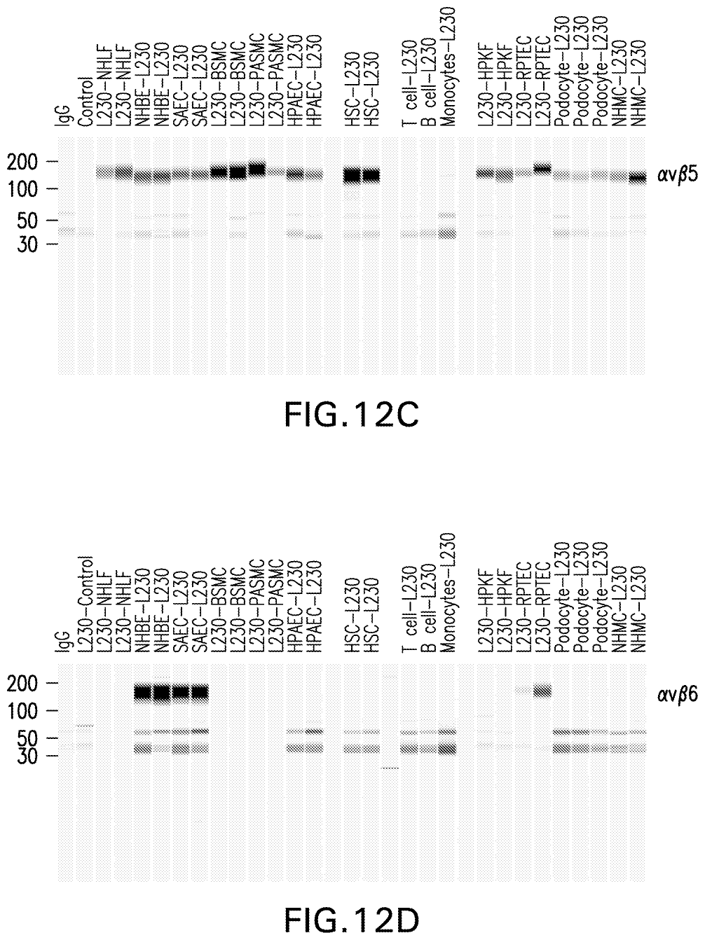

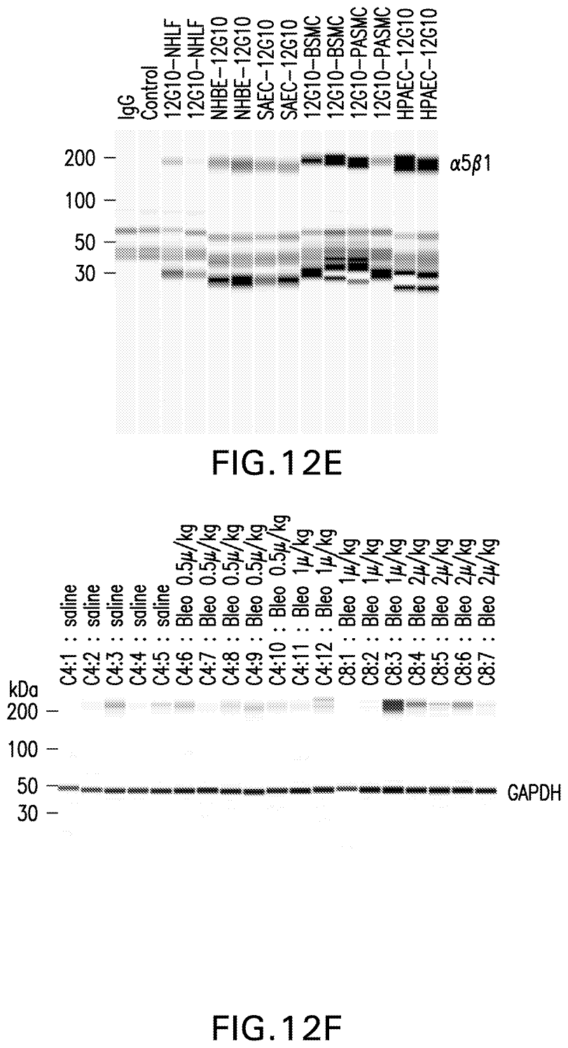

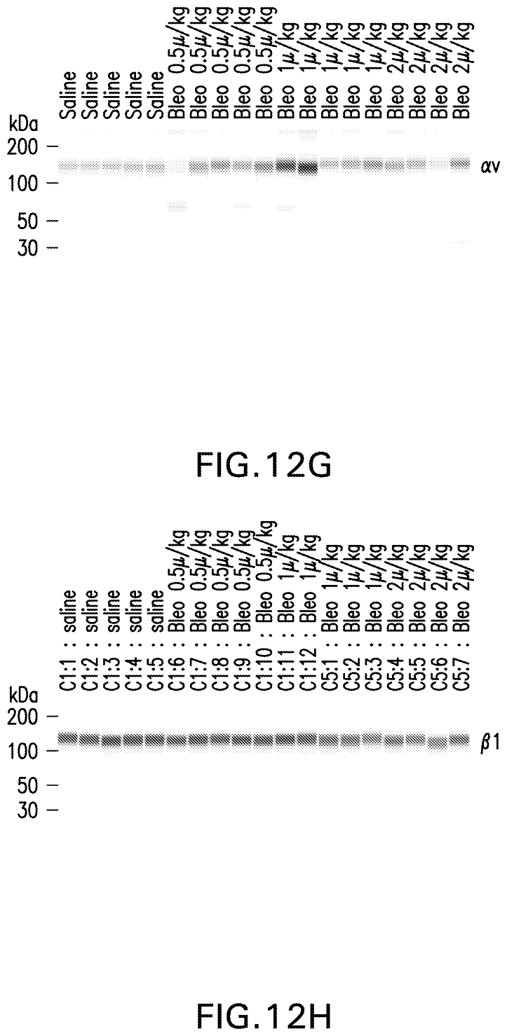

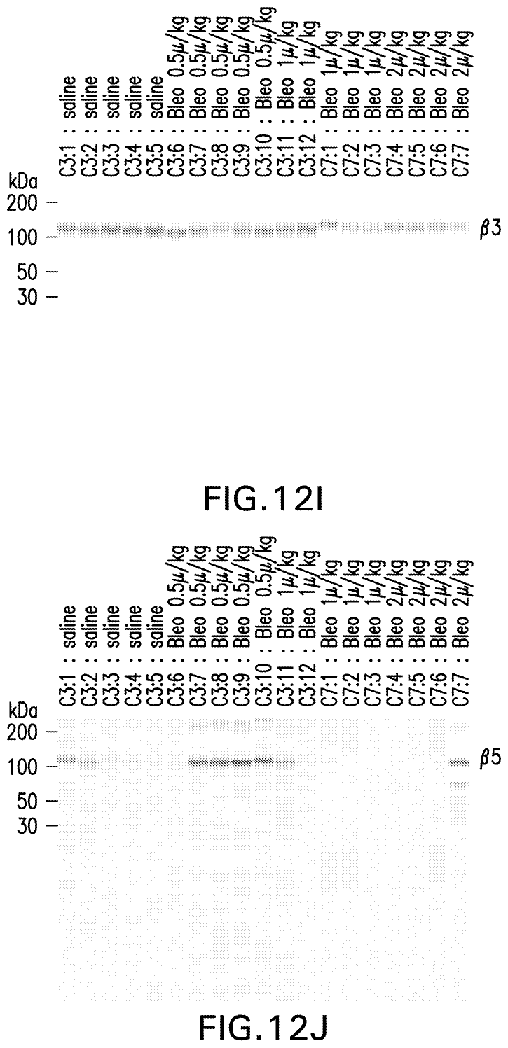

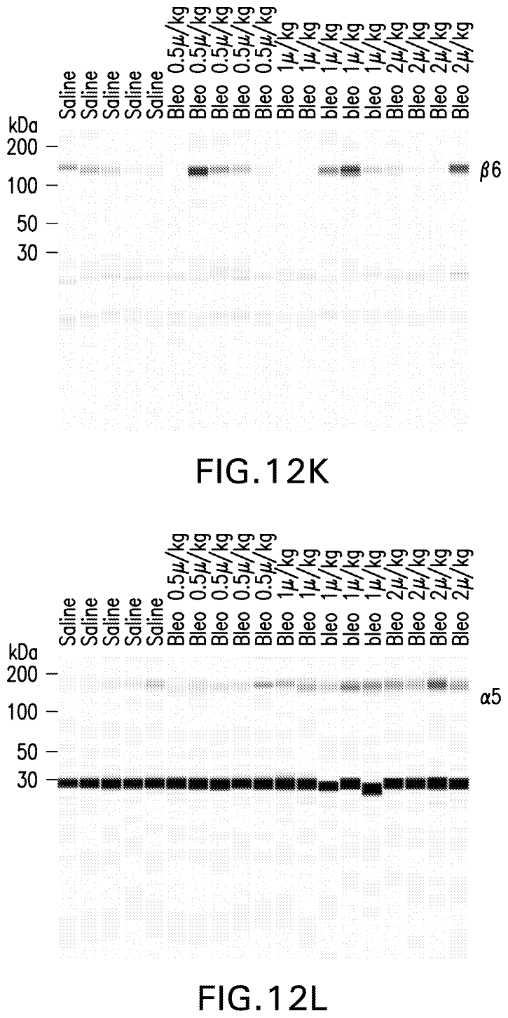

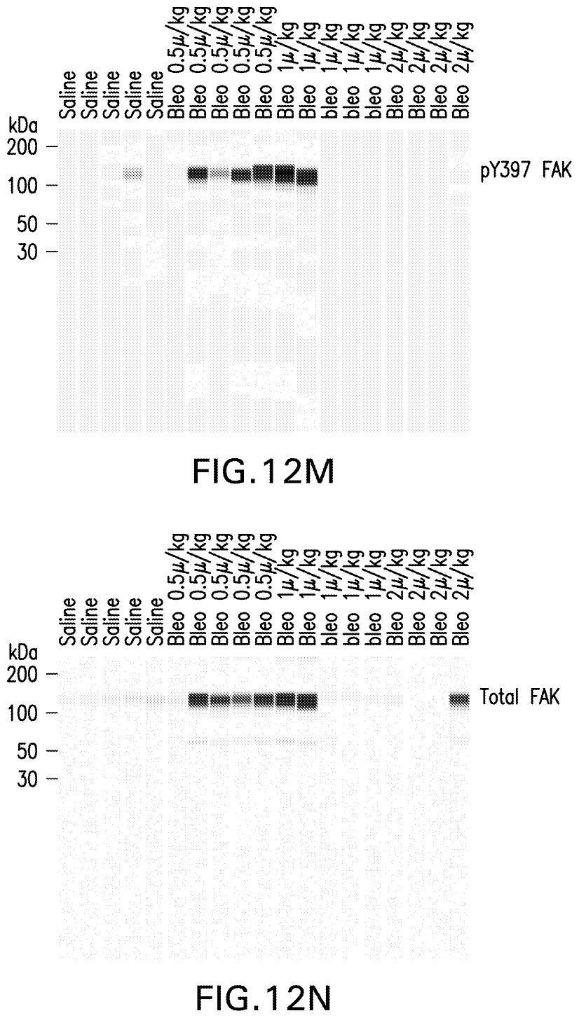

Ab-31 blocks integrin-mediated cell adhesion. Previous studies utilized cell adhesion assays to determine integrin subtype selectivity and cellular function (Reed, N. I. et al., Sci. Transl. Med. 7, 288 (2015); Hall et al, Biochem. Pharmacol. 117, 88-96 (2016)). The cell-based assay measures the binding of ligand to integrins that are endogenously present or over-expressed on the cell surface. Compared to in vitro functional assays using recombinant protein, cell adhesion results are more reflective of native integrin-ligand binding conformation. We generated CHOK1 stable cell lines that expressed various human and mouse αvβ× integrins (FIG. 9). Both αvβ1 and α5β1 can function as fibronectin receptors in cell adhesion assay (Wu, et al., J. Biol. Chem. 268, 21883-21888 (1993); Zhang et al., J. Cell Biol. 122, 235-242 (1993)). To delineate the role of αvβ1 in cell adhesion, we deleted endogenous hamster α5 gene in CHOK1 cells via CRISPR knockout/KO technology, and subsequently overexpressed αvβ1 to generate a CHOK1-α5KO-αvβ1 stable line. We next examined the effect of MK-0429 and our top monoclonal antibody clone, Ab-31, on the adhesion of cells to fibronectin or vitronectin matrix. MK-0429 showed little inhibitory activity in CHOK1 parental cells on fibronectin; however, it potently reduced the adhesion of mouse αvβ1-expressing CHOK1-α5KO cells (FIG. 11). Cell adhesion of mouse αvβ3 and αvβ5 on a vitronectin matrix were also decreased upon MK-0429 treatment. Similarly, Ab-31 significantly inhibited mouse αvβ1, αvβ3, and αvβ5 integrin-mediated cell adhesion, with IC₅₀s of 1.5 nM, 1.0 nM, and 5.6 nM respectively (FIG. 5A). The cell-based assays showed that Ab-31 is a potent αv-integrin inhibitor in a setting that resembles native integrin conformation.

### Example 5

Ab-31 substantially inhibits integrin-mediated latent TGFβ activation. The epithelium-specific αvβ6 integrin blocks the activation of latent TGFβ (Munger et al., Cell 96, 319-328 (1999); Dong et al., Nat. Struct. Mol. Biol. 21, 1091-1096 (2014)), a major mechanism-of-action for αv-integrin to modulate fibrosis. Rifkin et al. transfected mink lung epithelial cells with a firefly luciferase reporter under the control of PAI-1 (plasminogen activator inhibitor-1) promoter (Mazzieri et al., Methods Mol. Biol. 142, 13-27 (2000)). The transcription of PAI-1 is tightly controlled by the TGFβ-Smad pathway (Hua et al., Proc. Natl. Acad. Sci. USA 96, 13130-13135 (1999); Yingling et al., Mol. Cell Biol. 17, 7019-7028 (1997)). When co-culturing these transfected mink lung epithelial cells (TMLC) with another cell type-expressing integrin, the luciferase activity is driven by the abundance of mature TGFβ presented in extracellular matrix and culture medium. This co-culturing system provides a sensitive measurement of latent TGFβ activation by integrins.

In addition to αvβ6 integrin, both αvβ1 and αvβ8 have been shown activating latent TGFβ (Reed, N. I. et al., Sci. Transl. Med. 7, 288 (2015); Mu et al., J. Cell. Biol. 157, 493-507 (2002); Campbell et al., Cell 180, 490-501 (2020)). We next examined the activity of our monoclonal antibody Ab-31 against TGFβ activation in the TMLC-integrin co-culture system. Notably, Ab-31 substantially blocked the activation of latent TGFβ by human and mouse αvβ1, αvβ6, and αvβ8 integrins, with IC₅₀ at 6.1 nM, 19.1 nM, 3.9 nM, 7.5 nM, 3.1 nM, and 13.5 nM, respectively (FIG. 5B). MK-0429 also demonstrated strong inhibitory effect at TGFβ activation except in a mouse αvβ8 co-culture assay. The control antibody, mAb-24, was potent in human αvβ1, αvβ6, and αvβ8 co-culture systems but elicited minimal activity against mouse integrins, which is consistent with its activities in AlphaLISA assays. Together, our results found that pan-integrin monoclonal antibody Ab-31 strongly inhibits integrin-mediated cellular functions, including cell adhesion and latent TGFβ activation.

### Example 6

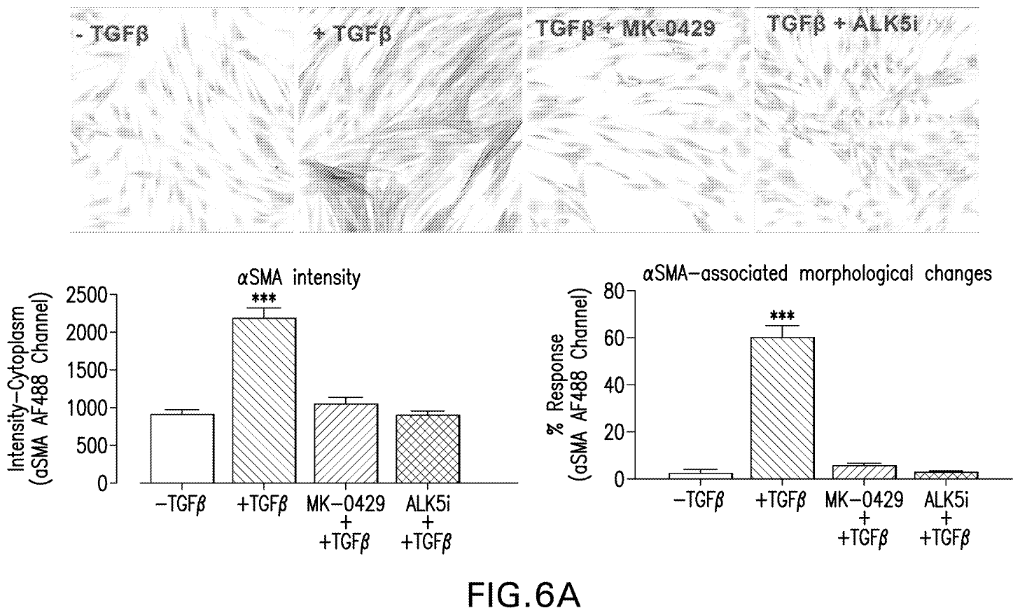

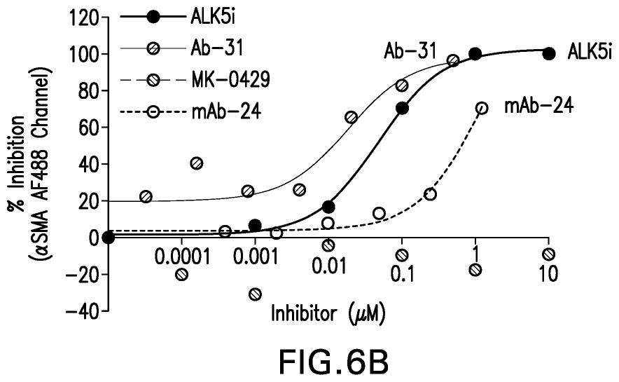

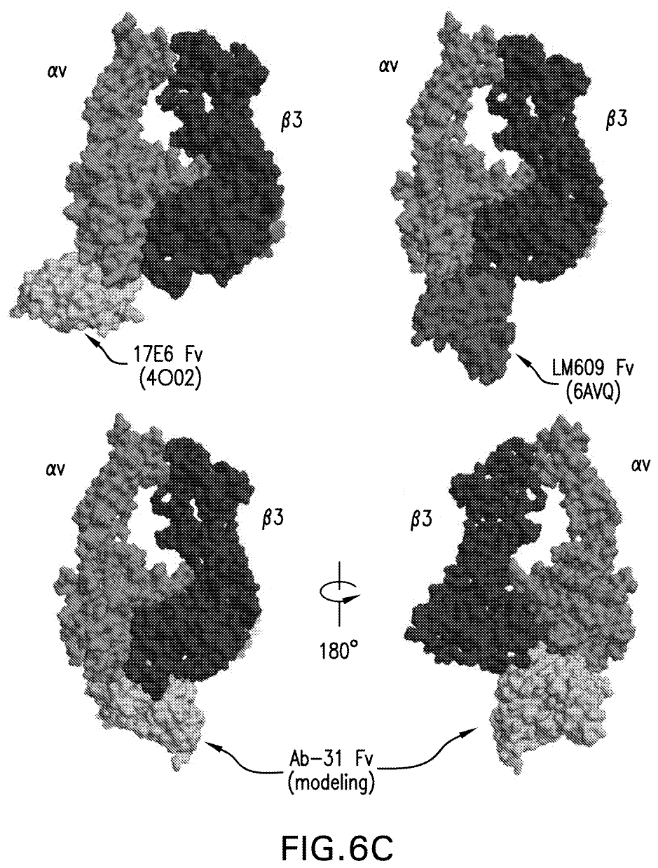

Ab-31 demonstrates potent inhibitory activity against TGFβ-induced αSMA expression. In addition to the inhibition of latent TGFβ activation, αv-integrin inhibitors also function downstream of TGFβ signaling (Zhou et al., Pharmacol. Res. Perspect. 5(5):e00354 (2017); Lygoe et al., Wound Repair Regen. 12, 461-470 (2004)). To further characterize the activities of our molecules in a fibrosis-relevant cell type, we next examined the expression of αSMA in primary human lung fibroblasts. Normal human lung fibroblasts were cultured, treated with TGFβ, and stained for αSMA by high-content phenotypic imaging analysis (FIG. 6A). Upon TGFβinduction, the fluorescence intensity of uSMA was vastly increased (FIG. 6A), and αSMA-associated cellular morphological changes were recognized as stimulated cells by the machine learning-based STAR software (Harmony high-content imaging and analysis software, PerkinElmer Inc.). Pretreating cells with MK-0429 or with SB-525334, a potent inhibitor of TGFβtype I receptor ALK5 (activin receptor-like kinase) (Grygielko et al., J. Pharmacol. Exp. Ther. 313, 943-951 (2005)) significantly reduced αSMA intensity and the percentage of stimulated cells following TGFβ treatment.

After establishing this αSMA phenotypic assay platform, we next examined the effects of integrin inhibitors on lung fibroblasts derived from IPF patients. As seen in the healthy human fibroblast assays, the expression of αSMA and percentage of stimulated cells among fibroblasts from IPF patients were also increased after TGFβ treatment (FIG. 6B). The ALK5 inhibitor, SB-525334, potently inhibited TGFβ-mediated αSMA induction in patient fibroblasts, with IC₅₀ of 56 z 22 nM. Notably, MK-0429 had minimal activity inhibiting αSMA induction even at a concentration of 10 M, suggesting that the network for αSMA regulation is distinct in IPF patient fibroblasts than that of normal human lung fibroblasts. The control antibody mAb-24 was also less effective at inhibiting αSMA expression with IC₅₀ above 1 μM. Interestingly, Ab-31 demonstrated a strong dose-dependent inhibition of uSMA intensity and associated morphological changes with IC₅₀ of 33±21 nM, similar to that of the ALK5 inhibitor (FIG. 6B). It is noteworthy that Ab-31 was ineffective in normal human lung fibroblasts, indicating that this molecule may preferentially recognize integrin confirmation under the fibrotic state. Our data demonstrate that Ab-31 was a potent inhibitor of TGFβsignaling in IPF patient lung fibroblasts and exhibits strong anti-fibrotic activity in cell-based assays.

### Example 7

Structural modeling of integrin-antibody complex. Over the past 20 years, a diverse set of integrin monoclonal antibodies have been discovered and extensively characterized that can elicit inhibitory, stimulatory, or neutral activity upon integrin binding (Byron et al., J. Cell Sci. 122, 4009-4011 (2009)). Integrin antibodies are also known to recognize active conformational epitope, such as 12G10 and 9EG7 clones of β1-integrin antibodies (Humphries et al., J. Biol. Chem. 280, 10234-10243 (2005); Mould et al., Lett. 363, 118-122 (1995); Lenter et al., Proc. Natl. Acad. Sci. USA 90, 9051-9055 (1993)). To further understand the mechanism of action of our antibody, we next set out to predict how it interacts with integrins.

The first crystal structure of integrin heterodimer was solved in 2001 and revealed a bent inactive confirmation of αvβ3 integrin ectodomains (Xiong et al., Science 294, 339-345 (2001)). Since then, several integrin-antibody complex structures have been determined and have provided valuable information in regard to their regulatory function. Antibody 17E6 (Abituzumab) is a therapeutic pan-av integrin antibody that has been tested in multiple Phase 2 clinical trials in cancer patients and systemic sclerosis patients with interstitial lung disease (Elez, et al., Ann. Oncol. 26, 132-140 (2015)) (clinicaltrials.gov identifier NCT02745145). The crystal structure of the 17E6 Fab fragment in complex with αvβ3 revealed that the antibody exclusively bound to the αv subunit and helped to establish a potential allosteric inhibition mechanism via steric hindrance (PDB: 4002) (Mahalingam et al., J. Biol. Chem. 289, 13801-13809 (2014)). As illustrated in the left panel of FIG. 6C, the variable region (Fv) of 17E6 binds to an epitope outside the ligand binding interface of the α- and β-subunits. In contrast, LM609 is an αvβ-specific blocking antibody of which the humanized variants, Vitaxin and Etaracizumab (Abegrin), have been tested in several clinical trials for oncology indications (Wu et al., Proc. Natl. Acad. Sci. USA 95, 6037-6042 (1998); Gutheil et al., Clin. Cancer Res. 6, 3056-3061 (2000); Veeravagu et al., Clin. Cancer Res. 14, 7330-7339 (2008)). A combination of crystallographic and cryo-EM work from Borst et al. found that LM609 bound to the apex of the integrin headpiece close to but without directly obstructing the RGD-binding site (Borst et al., Structure 25, 1732-1739 (2017)). The structure of LM609 Fv fragment in complex with αvβ3 integrin is illustrated in the second left panel of FIG. 6C, with an epitope locating at the interface between the α- and β-subunits. Compared to either of these characterized antibodies, Ab-31, as well as other top clones from our screen, represented a unique class of pan-integrin antibodies with human and mouse cross-reactivity.

To better understand the mechanism of integrin inhibition by Ab-31, we set out to model the Ab-31-integrin complex through homology modeling and docking. Using the antibody Fv sequence, we generated a series of 10 models for the Fv structure of Ab-31 using MOE (Chemical Computing Group). The lowest energy structure was then docked against the known structure of the known αvβ3 structure (see Molecular Modeling in General Methods). Following visualization and filtering of potential poses against known experimental variants, the final pose shown in FIG. 6C was determined. Interestingly, the model predicted that Ab-31 bound directly at the interface between α- and β-subunits and somewhat blocked access to the RGD-ligand binding site (FIG. 6C, right panels), indicating a distinct mode of action compared to 17E6 and LM609. This model is consistent with our epitope binning findings that Ab-31 had an epitope that did not overlap with other integrin antibodies.

The preferential effect of Ab-31 on IPF patient lung fibroblasts over normal human lung fibroblasts suggests that this antibody may selectively recognize either an active or diseased-associated integrin conformational state, and may provide an improved therapeutic index over currently available integrin inhibitors.

TABLE 14
Sequences

| SEQ | | |
|---|---|---|
| ID | | |
| NO: | Description | Sequence |
| 1 | Ab-29 VH-CDR 1 | SNSYYWG |
| 2 | Ab-29 VH-CDR 2 | SIYYSGSTYYNPSLKS |
| 3 | Ab-29 VH-CDR 3 | VRRVAGRGDSYYYYYGMDV |
| 4 | Ab-29 VL-CDR 1 | RASQGISRWLA |
| 5 | Ab-29 VL-CDR 2 | AASSLQS |
| 6 | Ab-29 VL-CDR 3 | QQAISHPWT |
| 7 | Ab-30 VH-CDR 1 | SGYYWG |
| 8 | Ab-30 VH-CDR 2 | SIYHSGNTYYNPSLKS |
| 9 | Ab-30 VH-CDR 3 | VPRYSRGDAYYYYGMDV |
| 10 | Ab-30 VL-CDR 1 | RASQSISSWLA |
| 11 | Ab-30 VL-CDR 2 | KASSLES |
| 12 | Ab-30 VL-CDR 3 | QQVKIYPPYT |
| 13 | Ab-31 VH-CDR 1 | SGYYWG |
| 14 | Ab-31 VH-CDR 2 | SIYHSGNTYYNPSLKS |
| 15 | Ab-31 VH-CDR 3 | VPRYSRGDAYYYYGMDV |
| 16 | Ab-31 VL-CDR 1 | RASQSIGSWLA |
| 17 | Ab-31 VL-CDR 2 | KASSLES |
| 18 | Ab-31 VL-CDR 3 | QQSGPYPWT |
| 19 | Ab-32 VH-CDR 1 | SGYYWG |
| 20 | Ab-32 VH-CDR 2 | SIYHSGNTYYNPSLKS |
| 21 | Ab-32 VH-CDR 3 | VPRYSRGDAYYYYGMDV |
| 22 | Ab-32 VL-CDR 1 | RASQSVRSSYLA |
| 23 | Ab-32 VL-CDR 2 | GASSRAT |
| 24 | Ab-32 VL-CDR 3 | QQAGLVFT |
| 25 | Ab-33 VH-CDR 1 | NYAIS |
| 26 | Ab-33 VH-CDR 2 | GIIPIFGTANYAQKFQG |
| 27 | Ab-33 VH-CDR 3 | APRSARGDMYYYYYGMDV |
| 28 | Ab-33 VL-CDR 1 | RASQSIGSWLA |
| 29 | Ab-33 VL-CDR 2 | KASSLES |
| 30 | Ab-33 VL-CDR 3 | QQYGRYPWT |
| 31 | Ab-29 VH | ELQLQESGPGLVKPSETLSLTCTVSGGSISSNSYYWGWIR |
| | | QPPGKGLEWIGSIYYSGSTYYNPSLKSRVTISVDTSKNQF |
| | | SLKLSSVTAADTAVYYCARVRRVAGRGDSYYYYYGMDV |
| | | WGQGTTVTVSS |
| 32 | Ab-29 VL | DIQMTQSPSSVSASVGDRVTITCRASQGISRWLAWYQQK |
| | | PGKAPKLLIYAASSLQSGVPSRFSGSGSGTDFTLTISSL |
| | | QPEDFATYYCQQAISHPWTFGGGTKVEIK |
| 33 | Ab-30 VH | EVQLQESGPGLVKPSETLSLTCAVSGYSISSGYYWGWIRQ |
| | | PPGKGLEWIGSIYHSGNTYYNPSLKSRVTISVDTSKNQFS |
| | | LKLSSVTAADTAVYYCARVPRYSRGDAYYYYGMDVWGQ |
| | | GTTVTVSS |
| 34 | Ab-30 VL | DIQMTQSPSTLSASVGDRVTITCRASQSISSWLAWYQQKP |
| | | GKAPKLLIYKASSLESGVPSRFSGSGSGTEFTLTISSLQP |
| | | DDFATYYCQQVKIYPPYTFGGGTKVEIK |
| 35 | Ab-31 VH | EVQLQESGPGLVKPSETLSLTCAVSGYSISSGYYWGWIRQ |
| | | PPGKGLEWIGSIYHSGNTYYNPSLKSRVTISVDTSKNQFSL |
| | | KLSSVTAADTAVYYCARVPRYSRGDAYYYYGMDVWGQ |
| | | GTTVTVSS |
| 36 | Ab-31 VL | DIQMTQSPSTLSASVGDRVTITCRASQSIGSWLAWYQQKP |
| | | GKAPKLLIYKASSLESGVPSRFSGSGSGTEFTLTISSLQPD |
| | | DFATYYCQQSGPYPWTFGGGTKVEIK |
| 37 | Ab-32 VH | EVQLQESGPGLVKPSETLSLTCAVSGYSISSGYYWGWIRQ |
| | | PPGKGLEWIGSIYHSGNTYYNPSLKSRVTISVDTSKNQFSL |
| | | KLSSVTAADTAVYYCARVPRYSRGDAYYYYGMDVWGQ |
| | | GTTVTVSS |
| 38 | Ab-32 VL | EIVLTQSPGTLSLSPGERATLSCRASQSVRSSYLAWYQQK |
| | | PGQAPRLLIYGASSRATGIPDRFSGSGSGTDFTLTISRLEPE |
| | | DFAVYYCQQAGLVFTFGGGTKVEIK |
| 39 | Ab-33 VH | EVQLVQSGAEVKKPGSSVKVSCKASGGTFSNYAISWVRQ |
| | | APGQGLEWMGGIIPIFGTANYAQKFQGRVTITADESTSTA |
| | | YMELSSLRSEDTAVYYCARAPRSARGDMYYYYYGMDV |
| | | WGQGTTVTVSS |
| 40 | Ab-34 VL | DIQMTQSPSTLSASVGDRVTITCRASQSIGSWLAWYQQKP |
| | | GKAPKLLIYKASSLESGVPSRFSGSGSGTEFTLTISSLQPD |
| | | DFATYYCQQYGRYPWTFGGGTKVEIK |
| 41 | Human IgG₁ HC | ASTKGPSVFPLAPSSKSTSGGTAALGCLVKDYFPEPVTVS |
| | constant domain | WNSGALTSGVHTFPAVLQSSGLYSLSSVVTVPSSSLGTQT |
| | | YICNVNHKPSNTKVDKKVEPKSCDKTHTCPPCPAPELLG |
| | | GPSVFLFPPKPKDTLMISRTPEVTCVVVDVSHEDPEVKFN |
| | | WYVDGVEVHNAKTKPREEQYNSTYRVVSVLTVLHQDW |
| | | LNGKEYKCKVSNKALPAPIEKTISKAKGQPREPQVYTLPP |
| | | SRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 42 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQT |
| | (E233A/L235A) | YICNVNHKPSNTKVDKKVEPKSCDKTHTCPPCPAPALAG |
| | | GPSVFLFPPKPKDTLMISRTPEVTCVVVDVSHEDPEVKFN |
| | | WYVDGVEVHNAKTKPREEQYNSTYRVVSVLTVLHQDW |
| | | LNGKEYKCKVSNKALPAPIEKTISKAKGQPREPQVYTLPP |
| | | SRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 43 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQT |
| | | YICNVNHKPSNTKVDKKVEPKSCDKTHTCPPCPAPEAAG |
| | | GPSVFLFPPKPKDTLMISRTPEVTCVVVSVSHEDPEVKFN |
| | (L234A L235A | WYVDGVEVHNAKTKPREEQYNSTYRVVSVLTVLHQDW |
| | D265S) | LNGKEYKCKVSNKALPAPIEKTISKAKGQPREPQVYTLPP |
| | | SRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 44 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQT |
| | (L234A L235A | YICNVNHKPSNTKVDKKVEPKSCDKTHTCPPCPAPEAAG |
| | P329G) | GPSVFLFPPKPKDTLMISRTPEVTCVVVSVSHEDPEVKFN |
| | | WYVDGVEVHNAKTKPREEQYNSTYRVVSVLTVLHQDW |
| | | LNGKEYKCKVSNKALGAPIEKTISKAKGQPREPQVYTLPP |
| | | SRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 45 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQT |
| | (L235E) | YICNVNHKPSNTKVDKKVEPKSCDKTHTCPPCPAPELEG |
| | | GPSVFLFPPKPKDTLMISRTPEVTCVVVDVSHEDPEVKFN |
| | | WYVDGVEVHNAKTKPREEQYNSTYRVVSVLTVLHQDW |
| | | LNGKEYKCKVSNKALPAPIEKTISKAKGQPREPQVYTLPP |
| | | SRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 46 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQT |
| | (D265A) | YICNVNHKPSNTKVDKKVEPKSCDKTHTCPPCPAPELLG |
| | | GPSVFLFPPKPKDTLMISRTPEVTCVVVAVSHEDPEVKFN |
| | | WYVDGVEVHNAKTKPREEQYNSTYRVVSVLTVLHQDW |
| | | LNGKEYKCKVSNKALPAPIEKTISKAKGQPREPQVYTLPP |
| | | SRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 47 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQT |
| | (D265A N297G) | YICNVNHKPSNTKVDKKVEPKSCDKTHTCPPCPAPELLG |
| | | GPSVFLFPPKPKDTLMISRTPEVTCVVVAVSHEDPEVKFN |
| | | WYVDGVEVHNAKTKPREEQYGSTYRVVSVLTVLHQDW |
| | | LNGKEYKCKVSNKALPAPIEKTISKAKGQPREPQVYTLPP |
| | | SRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 48 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQT |
| | (N297X, wherein | YICNVNHKPSNTKVDKKVEPKSCDKTHTCPPCPAPELLG |
| | X is any amino | GPSVFLFPPKPKDTLMISRTPEVTCVVVDVSHEDPEVKFN |
| | acid other than N) | WYVDGVEVHNAKTKPREEQYXSTYRVVSVLTVLHQDW |
| | | LNGKEYKCKVSNKALPAPIEKTISKAKGQPREPQVYTLPP |
| | | SRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 49 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQT |
| | (N297A/D356E/ | YICNVNHKPSNTKVDKKVEPKSCDKTHTCPPCPAPELLG |
| | L358M) | GPSVFLFPPKPKDTLMISRTPEVTCVVVDVSHEDPEVKFN |
| | | WYVDGVEVHNAKTKPREEQYASTYRVVSVLTVLHQDW |
| | | LNGKEYKCKVSNKALPAPIEKTISKAKGQPREPQVYTLPP |
| | | SREEMTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 50 | Human LC Kappa | RTVAAPSVFIFPPSDEQLKSGTASVVCLLNNFYPREAKVQ |
| | constant domain | WKVDNALQSGNSQESVTEQDSKDSTYSLSSTLTLSKADY |
| | | EKHKVYACEVTHQGLSSPVTKSFNRGEC |
| 51 | Leader peptide A | MSVPTQVLGLLLLWLTDARC |
| 52 | Leader peptide B | MEWSWVFLFFLSVTTGVHS |
| 53 | G4S | GGGGS |
| 54 | (G4S)n, n is 1, 2, | GGGGSGGGGS GGGGSGGGGS GGGGS |
| | 3, 4, or 5 | |
| 55 | (G4S)₃ | GGGGSGGGGSGGGGS |
| 56 | (G4S)n, n is 1 to | GGGGSGGGGS GGGGSGGGGS GGGGSGGGGS |
| | 10 peptide units | GGGGSGGGGS GGGGSGGGGS |
| 57 | (GGGS)₃ | GGGSGGGSGGGS |
| 58 | hexa-histidine tag | HHHHHH |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the language of the specification and the claims attached herein.

1. An integrin binder comprising:
(a) the six complementarity determining regions (CDRs) of an antibody having a heavy chain variable domain (V_{H}) comprising the amino acid sequence set forth in SEQ ID NO: 31 and a light chain variable domain (V_{L}) comprising the amino acid sequence set forth in SEQ ID NO: 32;
(b) the six CDRs of an antibody having a V_{H} comprising the amino acid sequence set forth in SEQ ID NO: 33 and a V_{L} comprising the amino acid sequence set forth in SEQ ID NO: 34;
(c) the six CDRs of an antibody having a V_{H} comprising the amino acid sequence set forth in SEQ ID NO: 35 and a V_{L} comprising the amino acid sequence set forth in SEQ ID NO: 36;
(d) the six CDRs of an antibody having a V_{H} comprising the amino acid sequence set forth in SEQ ID NO: 37 and a V_{L} comprising the amino acid sequence set forth in SEQ ID NO: 38; or,
(e) the six CDRs of an antibody having a V_{H} comprising the amino acid sequence set forth in SEQ ID NO: 39 and a V_{L} comprising the amino acid sequence set forth in SEQ ID NO: 40;
wherein in (a), (b), (c), (d) and (e) the CDRs are defined using the Kabat, Chothia, AbM, ImMunoGeneTics (IMGT), or Contact numbering scheme and
wherein the integrin binder binds human αvβ1, αvβ3, αvβ5, αvβ6, and αvβ8 integrins and mouse αvβ1, αvβ3, αvβ5, αvβ6, and αvβ8 integrins.

2. The integrin binder of claim 1, wherein
(a) the V_{H} comprises a CDR 1 comprising the amino acid sequence set forth in SEQ ID NO: 1, a CDR-2 comprising the amino acid sequence set forth in SEQ ID NO: 2, and a CDR 3 comprising the amino acid sequence set forth in SEQ ID NO: 3; and the V_{L} comprises a CDR 1 comprising the amino acid sequence set forth in SEQ ID NO: 4, a CDR 2 comprising the amino acid sequence set forth in SEQ ID NO: 5, and a CDR 3 comprising the amino acid sequence set forth in SEQ ID NO: 6;
(b) the V_{H} comprises a CDR 1 comprising the amino acid sequence set forth in SEQ ID NO: 7, a CDR-2 comprising the amino acid sequence set forth in SEQ ID NO: 8, and a CDR 3 comprising the amino acid sequence set forth in SEQ ID NO: 9; and the V_{L} comprises a CDR 1 comprising the amino acid sequence set forth in SEQ ID NO: 10, a CDR 2 comprising the amino acid sequence set forth in SEQ ID NO: 11, and a CDR 3 comprising the amino acid sequence set forth in SEQ ID NO: 12;
(c) the V_{H} comprises a CDR 1 comprising the amino acid sequence set forth in SEQ ID NO: 13, a CDR-2 comprising the amino acid sequence set forth in SEQ ID NO: 14, and a CDR 3 comprising the amino acid sequence set forth in SEQ ID NO: 15; and the V_{L} comprises a CDR 1 comprising the amino acid sequence set forth in SEQ ID NO: 16, a CDR 2 comprising the amino acid sequence set forth in SEQ ID NO: 17, and a CDR 3 comprising the amino acid sequence set forth in SEQ ID NO: 18;
(d) the V_{H} comprises a CDR 1 comprising the amino acid sequence set forth in SEQ ID NO: 19, a CDR-2 comprising the amino acid sequence set forth in SEQ ID NO: 20, and a CDR 3 comprising the amino acid sequence set forth in SEQ ID NO: 21; and the V_{L} comprises a CDR 1 comprising the amino acid sequence set forth in SEQ ID NO: 22, a CDR 2 comprising the amino acid sequence set forth in SEQ ID NO: 23, and a CDR 3 comprising the amino acid sequence set forth in SEQ ID NO: 24; and
(e) the V_{H} comprises a CDR 1 comprising the amino acid sequence set forth in SEQ ID NO: 25, a CDR-2 comprising the amino acid sequence set forth in SEQ ID NO: 26, and a CDR 3 comprising the amino acid sequence set forth in SEQ ID NO: 27; and the V_{L} comprises a CDR 1 comprising the amino acid sequence set forth in SEQ ID NO: 28, a CDR 2 comprising the amino acid sequence set forth in SEQ ID NO: 29, and a CDR 3 comprising the amino acid sequence set forth in SEQ ID NO: 30.

3. The integrin binder of claim 1 comprising
(a) a heavy chain variable domain (V_{H}) comprising an amino acid sequence with at least 90% identity to the amino acid sequence set forth in SEQ ID NO: 31 and a light chain variable domain (V_{L}) comprising an amino acid sequence with at least 90% identity to the amino acid sequence set forth in SEQ ID NO: 32, wherein the V_{H} comprises a CDR 1 comprising the amino acid sequence set forth in SEQ ID NO: 1, a CDR-2 comprising the amino acid sequence set forth in SEQ ID NO: 2, and a CDR 3 comprising the amino acid sequence set forth in SEQ ID NO: 3, and the V_{L} comprises a CDR 1 comprising the amino acid sequence set forth in SEQ ID NO: 4, a CDR 2 comprising the amino acid sequence set forth in SEQ ID NO: 5, and a CDR 3 comprising the amino acid sequence set forth in SEQ ID NO: 6;
(b) a V_{H} comprising an amino acid sequence with at least 90% identity to the amino acid sequence set forth in SEQ ID NO: 33 and a V_{L} comprising an amino acid sequence with at least 90% identity to the amino acid sequence set forth in SEQ ID NO: 34, wherein the V_{H} comprises a CDR 1 comprising the amino acid sequence set forth in SEQ ID NO: 7, a CDR-2 comprising the amino acid sequence set forth in SEQ ID NO: 8, and a CDR 3 comprising the amino acid sequence set forth in SEQ ID NO: 9, and the V_{L} comprises a CDR 1 comprising the amino acid sequence set forth in SEQ ID NO: 10, a CDR 2 comprising the amino acid sequence set forth in SEQ ID NO: 11, and a CDR 3 comprising the amino acid sequence set forth in SEQ ID NO: 12;
(c) a V_{H} comprising an amino acid sequence with at least 90% identity to the amino acid sequence set forth in SEQ ID NO: 35 and a V_{L} comprising an amino acid sequence with at least 90% identity to the amino acid sequence set forth in SEQ ID NO: 36, wherein the V_{H} comprises a CDR 1 comprising the amino acid sequence set forth in SEQ ID NO: 13, a CDR-2 comprising the amino acid sequence set forth in SEQ ID NO: 14, and a CDR 3 comprising the amino acid sequence set forth in SEQ ID NO: 15, and the V_{L} comprises a CDR 1 comprising the amino acid sequence set forth in SEQ ID NO: 16, a CDR 2 comprising the amino acid sequence set forth in SEQ ID NO: 17, and a CDR 3 comprising the amino acid sequence set forth in SEQ ID NO: 18;
(d) a V_{H} comprising an amino acid sequence with at least 90% identity to the amino acid sequence set forth in SEQ ID NO: 37 and a V_{L} comprising an amino acid sequence with at least 90% identity to the amino acid sequence set forth in SEQ ID NO: 38, wherein the V_{H} comprises a CDR 1 comprising the amino acid sequence set forth in SEQ ID NO: 19, a CDR-2 comprising the amino acid sequence set forth in SEQ ID NO: 20, and a CDR 3 comprising the amino acid sequence set forth in SEQ ID NO: 21, and the V_{L} comprises a CDR 1 comprising the amino acid sequence set forth in SEQ ID NO: 22, a CDR 2 comprising the amino acid sequence set forth in SEQ ID NO: 23, and a CDR 3 comprising the amino acid sequence set forth in SEQ ID NO: 24; or
(e) a V_{H} comprising an amino acid sequence with at least 90% identity to the amino acid sequence set forth in SEQ ID NO: 39 and a V_{L} comprising an amino acid sequence with at least 90% identity to the amino acid sequence set forth in SEQ ID NO: 40, wherein the V_{H} comprises a CDR 1 comprising the amino acid sequence set forth in SEQ ID NO: 25, a CDR-2 comprising the amino acid sequence set forth in SEQ ID NO: 26, and a CDR 3 comprising the amino acid sequence set forth in SEQ ID NO: 27, and the V_{L} comprises a CDR 1 comprising the amino acid sequence set forth in SEQ ID NO: 28, a CDR 2 comprising the amino acid sequence set forth in SEQ ID NO: 29, and a CDR 3 comprising the amino acid sequence set forth in SEQ ID NO: 30.

4. The integrin binder of claim 3 comprising
(a) a heavy chain variable domain (V_{H}) comprising the amino acid sequence set forth in SEQ ID NO: 31 and a light chain variable domain (V_{L}) comprising the amino acid sequence set forth in SEQ ID NO: 32;
(b) a V_{H} comprising the amino acid sequence set forth in SEQ ID NO: 33 and a V_{L} comprising the amino acid sequence set forth in SEQ ID NO: 34;
(c) a V_{H} comprising the amino acid sequence set forth in SEQ ID NO: 35 and a V_{L} comprising the amino acid sequence set forth in SEQ ID NO: 36;
(d) a V_{H} comprising the amino acid sequence set forth in SEQ ID NO: 37 and a V_{L} comprising the amino acid sequence set forth in SEQ ID NO: 38; or
(e) a V_{H} comprising the amino acid sequence set forth in SEQ ID NO: 39 and a V_{L} comprising the amino acid sequence set forth in SEQ ID NO: 40.

5. (canceled)

6. The integrin binder of claim 1, wherein the integrin binder comprises an antibody comprising a heavy chain constant domain of the IgG1 or IgG4 isotype and a light chain constant domain of the human kappa or human lambda isotype.

7-9. (canceled)

10. The integrin binder of claim 6, wherein the heavy chain constant domain of the IgG1 isotype comprises an Fc domain comprising one or more mutations that render the constant domain effector-silent.

11-14. (canceled)

15. The integrin binder of claim 1, wherein the integrin binder is an antigen-binding fragment of an antibody selected from the group consisting of a Fab fragment, a Fab′ fragment, a F(ab′)₂ fragment, an Fv region, and an ScFv.

16-17. (canceled)

18. A composition comprising the integrin binder of claim 1 and a pharmaceutically acceptable carrier or diluent.

19. A method for treating cancer or fibrosis in an individual in need thereof comprising administering to the individual a therapeutically effective amount of the integrin binder of claim 1 to treat the cancer or fibrosis.

20. The method of claim 19, wherein the fibrosis is idiopathic pulmonary fibrosis.

21-24. (canceled)

25. A combination therapy for treating cancer or fibrosis comprising the integrin binder of claim 1 and a therapeutic agent.

26. The combination therapy of claim 25, wherein the therapeutic agent is a chemotherapy agent or a therapeutic antibody.

27. A nucleic acid molecule encoding the integrin binder of claim 1.

28. An expression vector comprising the nucleic acid molecule of claim 27.

29. A host cell comprising the expression vector of claim 28.

30. A method for producing an integrin binder comprising (a) providing a host cell of claim 29; (b) cultivating the host cell in a medium under conditions suitable for expressing the integrin binder; and (c) isolating the integrin binder from the medium.

31. The integrin binder of claim 1 conjugated to a detectable moiety.

32. The integrin binder of claim 31, wherein the detectable moiety is detectable by magnetic resonance imaging (MRI) or by X-ray imaging.

33. A method for detecting integrin expression on the surface of cells in an individual comprising administering to the individual the integrin binder of claim 31 and detecting the cells in the individual bound to the integrin binder.

34. A method for treating idiopathic pulmonary fibrosis in an individual in need of the treatment comprising administering to the individual a therapeutically effective amount of (3S)-3-(6-methoxypyridin-3-yl)-3-[2-oxo-3-[3-(5,6,7,8-tetrahydro-1,8-naphthyridin-2-yl)propyl]imidazolidin-1-yl]propanoic acid to treat the idiopathic pulmonary fibrosis.

35-36. (canceled)
